(19) United States
(12) Patent Application Publication
(10) Pub. No.: US 2023/0071189 A1
(43) Pub. Date: Mar. 9, 2023

(54) BACTERIAL QUANTIFICATION METHOD

(71) Applicant: Microbio Pty Ltd, Westlake, Queensland (AU)

(72) Inventor: Flavia Huygens, Westlake, Queensland (AU)

(73) Assignee: Microbio Pty Ltd, Westlake, Queensland (AU)

(21) Appl. No.: 17/794,228

(22) PCT Filed: Jan. 22, 2021
(86) PCT No.: PCT/AU2021/050038

§ 371 Date: Jul. 20, 2022

(30) Foreign Application Priority Data
Jan. 22, 2020 (AU) 2020900167

(51) Int. Cl. C12Q 1/689

(52) U.S. Cl. CPC C12Q 1/689; C12Q 2600/118; C12Q 1/686

(57) ABSTRACT

The present invention relates to methods and agents for determining the quantity or concentration of a bacterium in a sample. The method may include the steps of: (a) amplifying a target nucleic acid of the bacterium from genetic material obtained from the sample to form an amplification product, wherein the target nucleic acid comprises at least a portion of a bacterial 16S rRNA gene; (b) measuring a quantity or concentration of the amplification product; (c) calculating a quantity or concentration of the target nucleic acid in the sample by comparing the quantity or concentration of the amplification product with a reference level thereof; and (d) determining a copy number of the bacterial 16S rRNA gene in the sample from the quantity or concentration of the target nucleic acid therein, the copy number being a function of or correlated to the quantity of the bacterium in the sample. In other embodiments, the invention relates to methods of determining a prognosis for an infection by a bacterium in a subject, to methods of treating an infection or of evaluating treatment efficacy of an infection by a bacterium and to kits and assays.

### TECHNICAL FIELD

The present invention relates generally to methods and agents for quantifying bacteria particularly in a biological sample from a subject. The invention also features methods for the prognosis and treatment of bacterial infections based on the quantification methods of the present invention.

### BACKGROUND

Rapid and accurate quantification of bacteria in a sample, and more particularly biological samples, is highly desirable.

First and foremost, rapid and accurate quantification of bacteria in biological samples taken from, for example, septic patients can be of prognostic value and assist clinicians in their therapeutic decision making.

Rapid and accurate quantification may also assist in the implementation of effective control measures to manage, control, eradicate and/or eliminate bacteria in contaminated solutions, materials or foodstuffs, which may otherwise pose a threat to the wellbeing of organisms or the quality of production of the solutions, materials or foodstuffs.

Quantification is the ability to count the real number of bacteria in each sample. Traditionally for bacteria because they are so small, it was almost impossible to count each individual cell. To overcome this issue scientists and clinicians have developed two general methods for helping quantify the given number of pathogens in a sample. First, samples of interest (growth media, blood, urine, serum etc) were serially diluted onto plate assays. When the dilution was high enough it was possible to count the number of individual colonies growing on these plates. Secondly since it was impractical to count each individual cell without advanced microscopes, each individual colony was termed a colony forming unit (CFU). From this point on it has become standard in microbiology to define the number of cells with the unit CFU.

The gold standard for quantifying bacteria in samples taken from septic patients involves growing pathogens in specialised blood culture system. Blood (8-10 ml) is taken from patients who are suspected of having sepsis which is put into special growth media to grow the pathogen to detectable levels. Once the machine registers growth the media is plated out on various growth culture mediums for identification. The two growth phases required for this process have a profound impact on how long it takes to identify a pathogen: 12±10 hours to determine bacteria is present. The problem with this approach apart from being time consuming, is there is no opportunity to quantify the patient's initial pathogen level. Following the initial growth phase, it is close to impossible to accurately calculate the amount of bacterial cells/ml of blood. Ultimately, the number of colonies are counted on a plate to determine CFU, however this is highly inaccurate.

Additionally, the various molecular methods for detecting bacteria in samples taken from septic patients (e.g., those commercially available from T2 Biosystems and AusDiagnostics) generally involve no specific quantification of the pathogen detected as the methods only register the presence of the pathogen following high levels of amplification making any assumptions on initial CFU very inaccurate.

Hence, there is a recognized need for rapid and reliable techniques for accurate quantification of bacteria in a sample.

### SUMMARY OF INVENTION

In various aspects, the present invention is predicated in part on high conservation of the 16S (Svedberg unit) ribosomal RNA (16S rRNA) gene between prokaryotes, including bacteria and the multiple single nucleotide polymorphisms (SNPs) therein that may be useful in the identification and quantification of bacteria in a sample based on the bacteria's copy number of the 16S rRNA gene therein.

Generally speaking, prokaryotes, including bacteria, contain 16S rRNA, which is a component of the 30S small subunit of the prokaryotic ribosome. The 16S rRNA is approximately 1,500 nucleotides in length and encoded by the 16S rRNA gene (also referred to as 16S rDNA), which is generally part of a co-transcribed operon also containing the 23S and 5S rRNA genes. Although the DNA sequence of the 16S rRNA genes (and thus the RNA sequence of the 16S rRNA molecules) is highly conserved between prokaryotes, there are regions of variation (Weisberg W. G., et al., 1991). Relevant to the present invention, the gene copy numbers of the 16S rRNA gene per genome are typically species or strain-specific and consistent therein, but may vary from 1 up to 15 or more copies of this gene between bacterial species and strains (Klappenbach J A, et al., 2001). Exemplary bacterial 16S rRNA gene sequences are set forth in SEQ ID NOs:1-15 and 38-47.

According to a first aspect of the invention, there is provided a method of determining a quantity or concentration of a bacterium in a sample, said method includes the steps of:

(a) amplifying a target nucleic acid of the bacterium from genetic material obtained from the sample to form an amplification product, wherein the target nucleic acid comprises at least a portion of a bacterial 16S rRNA gene;

(b) measuring a quantity or concentration of the amplification product;

(c) calculating a quantity or concentration of the target nucleic acid in the sample by comparing the quantity or concentration of the amplification product with a reference level thereof; and

(d) quantifying the bacterium by determining a copy number of the bacterial 16S rRNA gene from the quantity or concentration of the target nucleic acid in the sample, the copy number being a function of or correlated to the quantity of the bacterium in the sample.

Suitably, the bacterial 16S rRNA gene is selected from those set forth in SEQ ID NOs:1-15 and 38-47 or a variant nucleotide sequence thereof having at least 90%, at least 91%, at least 92%, at least 93%, at least 94%, at least 95%, at least 96%, at least 97%, at least 98%, or at least 99% sequence homology or identity therewith.

Suitably, the target nucleic acid comprises one or a plurality of single nucleotide polymorphisms (SNPs) in the bacterial 16S rRNA gene. In one particular embodiment, the one or plurality of SNPs corresponding to at least one of positions 273, 378, 408, 412, 440, 488, 647, 653, 737, 755, 762 and 776 of the 16S rRNA gene set forth in SEQ ID NO: 1.

In another embodiment, the method of the present aspect further includes the step of generating the reference level from one or a plurality of control samples, such as by amplifying the target nucleic acid from the one or plurality of control samples, wherein the control samples comprise genetic material from a known quantity or concentration, such as that obtained or derived from the bacterium. In certain embodiments, amplifying the target nucleic acid from the one or plurality of control samples is performed substantially simultaneously or in parallel with step (a). In other embodiments, the one or plurality of control samples further comprise genetic material from one or a plurality of further bacteria.

In one embodiment, amplifying the target nucleic acid from genetic material of the sample and/or the one or plurality of control samples is conducted with a pair of primers that comprise at least one of SEQ ID NOs: 16-37 and 48-51.

In particular embodiments, amplifying the target nucleic acid from genetic material of the sample and/or the one or plurality of control samples comprises the use of quantitative PCR, semi-quantitative PCR, digital PCR, endpoint PCR, ligase chain reaction (LCR), Sanger sequencing, next generation sequencing or any combination thereof.

In certain embodiments, the method of the present aspect further includes the step of identifying the bacterium in the sample, such as by analysing the amplification product for the presence or absence of at least one SNP, such that bacterium in the sample is identified based on the presence or absence of the at least one SNP. By way of example, the at least one SNP can be in or corresponds to the 16S rRNA gene set forth in SEQ ID NO: 38. In other embodiments, the at least one SNP is at a position corresponding to at least one of positions 273, 378, 408, 412, 440, 488, 647, 653, 737, 755, 762 and 776 of the 16S rRNA gene set forth in SEQ ID NO: 1. Suitably, the bacterium is identified based on the presence of the at least one SNP.

In particular embodiments, the step of analysing the amplification product for the presence or the absence of the at least one SNP comprises the use of high resolution melt analysis, 5′ nuclease digestion, molecular beacons, oligonucleotide ligation, microarray, restriction fragment length polymorphism, antibody detection methods, direct sequencing or any combination thereof.

Suitably, the copy number is determined using the formula:

$$copy number = \frac{X ng * 6.0221 \times {10}^{23} molecules / mole}{( N * 660 g / mole ) * 1 \times {10}^{9} ng / g}$$

wherein X is the quantity of the amplification product and N is the length in nucleotides of the target nucleic acid.

In one embodiment, the sample is a biological sample taken from a subject. Suitably, the subject has an infection by the bacterium, such as sepsis.

According to a second aspect of the invention, there is provided a method of determining a prognosis for an infection by a bacterium in a subject, including the step of quantifying the bacterium in a biological sample from the subject according to the method of the first aspect to thereby evaluate the prognosis of the infection in the subject.

Depending upon whether a concentration or quantity of the bacterium in the biological sample, is altered, modulated or relatively high or low in the biological sample, the prognosis may be negative or positive. In this regard, a relatively high quantity or concentration or an increase in the quantity or concentration of the bacterium in the biological sample may indicate a negative prognosis for the subject, whilst a relatively low quantity or concentration or a decrease in the quantity or concentration of the bacterium in the biological sample may indicate a positive prognosis for the subject.

In one embodiment, the quantity or concentration of the bacterium is determined before, during and/or after a treatment, such as an antibiotic treatment.

In one embodiment, the prognosis is used, at least in part, to determine whether the subject would benefit from treatment of the infection.

In one embodiment, the prognosis is used, at least in part, to develop a treatment strategy for the subject.

In one embodiment, the prognosis is used, at least in part, to determine disease progression or recurrence in the subject.

According to a third aspect of the invention, there is provided a method of treating an infection by a bacterium in a subject including the steps of;

quantifying the bacterium in a biological sample from the subject according to the method of the first aspect; and

based on the quantification made, initiating, continuing, modifying or discontinuing a treatment of the infection.

According to a fourth aspect of the invention, there is provided a method of evaluating treatment efficacy of an infection by a bacterium in a subject including:

quantifying the bacterium in a biological sample from the subject according to the method of the first aspect; and

determining whether or not the treatment is efficacious according to whether said quantity of the bacterium in the subject's biological sample is reduced or absent.

Referring to the aforementioned aspects, the subject may have sepsis.

According to a fifth aspect of the invention, there is provided a kit or assay for quantifying a bacterium in a sample, said kit or assay comprising one or more reagents for performing the method according to first, second, third or fourth aspects and instructions for use.

In one embodiment, the kit or assay comprises at least one isolated probe, tool or reagent that is capable of identifying, partially identifying, or classifying at least one bacteria in a sample, wherein the probe, tool or reagent is capable of binding, detecting or identifying the presence or absence of at least one single nucleotide polymorphism (SNP), such as those described herein, in at least a portion of a bacterial 16S rRNA gene. In particular embodiments, the at least one SNP is at a position corresponding to at least one of positions 273, 378, 408, 412, 440, 488, 647, 653, 737, 755, 762 and 776 of the 16S rRNA gene set forth in SEQ ID NO: 1.

In one embodiment, said at least one isolated probe, tool or reagent is capable of discriminating between a sample that comprises at least one bacterium and a sample that does not comprise at least one bacterium.

In particular embodiments, the kit or assay is or comprises an array or microarray of oligonucleotide probes for identifying the bacterium and optionally one or a plurality of further bacteria in a sample, said probes comprising oligonucleotides which hybridize to at least one SNP in a 16S rRNA gene in the sample as broadly described above.

In another embodiment, the kit or assay is or comprises a biochip comprising a solid substrate and at least one oligonucleotide probe for identifying the bacterium and optionally one or a plurality of further bacteria in a sample, said at least one probe comprising an oligonucleotide which hybridizes to at least one SNP in a 16S rRNA gene in the sample as broadly described above.

In particular embodiments of the aforementioned aspects, the sample or biological sample is or comprises sputum, blood, cerebrospinal fluid and/or urine.

Features of the first to fifth aspects of the present invention, where appropriate, may be as described below.

In one embodiment, genetic material containing the target nucleic acid is extracted or obtained from the sample prior to analysis in the methods of the invention. It is envisaged that the nucleic acid may be extracted or obtained from the sample by any method or means known in the art.

The skilled artisan will appreciate that the step of amplifying the target nucleic acid of the bacterium may be performed by any method known in the art including, but not limited to quantitative PCR, semi-quantitative PCR, digital PCR, endpoint PCR, ligase chain reaction (LCR), Sanger sequencing, next generation sequencing or any combination thereof, using one or more oligonucleotides/primers that will amplify the target nucleic acid.

When a specific target nucleic acid is amplified by one of the above method and reaches a detectable amount having a detectable signal, such as a fluorescent signal, a rapid increase in signal intensity is observed. The number of cycles at this time is referred to as Threshold Cycle (hereinafter referred to as Ct value). In PCR methods, DNA is generally doubled every cycle, and DNA is amplified exponentially. As the initial amount of the amplification product of the target nucleic acid contained in the sample increases, the amount of a signal, such as fluorescence by a bound label, that can be detected with a smaller number of cycles is reached, so the Ct value decreases.

Broadly speaking, there is a linear relationship between the Ct value and the common logarithm of the initial target nucleic acid amount, and reference levels defining a calibration curve can be created based on this. In other words, one or more reference levels can be created by measuring the Ct value of a plurality of control samples having different DNA concentrations of the target nucleic acid by such amplification methods, and plotting, for example, the Ct value on the vertical axis and the initial DNA amount before starting PCR on the horizontal axis. This calibration curve represents the relationship between the quantity or concentration of the target nucleic acid in the sample and the Ct value, and the amount of DNA contained in the sample can then be readily determined from this relationship.

It will be understood that the copy number of the 16S rRNA gene may be determined or quantified by any method or algorithm known in the art, such as the dsDNA copy number calculator at https://cels.uri.edu/gsc/cdna.html. In this regard, this copy number calculator utilises the following algorithm:

$$copy number = \frac{X ng * 6.0221 \times {10}^{23} molecules / mole}{( N * 660 g / mole ) * 1 \times {10}^{9} ng / g}$$

wherein X is the quantity of the amplification product and N is the length in nucleotides of the target nucleic acid. This equation is based on the assumption that the average weight of a base pair is 660 Daltons, such that one mole of a base pair weighs about 660 g and that the molecular weight of any double stranded DNA template or amplification product can be estimated by from the product of its length in base pairs and 660. The inverse of the molecular weight is the number of moles of template present in one gram of material. Using Avogadro's number, 6.022×10²³ molecules/mole, the number of molecules of the template per gram can then be calculated (mol/g*molecules/mol=molecules/g). Finally, the number of molecules or copy number of the 16S rRNA gene in the sample can be estimated by multiplying by 1*10⁹ to convert to nanograms and then multiplying by the amount of template (in nanograms).

Suitably, quantifying the bacterium further comprises dividing the copy number of the bacterial 16S rRNA gene in the sample determined in step (d) by a gene copy number of the bacterial 16S rRNA gene in the bacterium. For example, the gene copy number of the bacterial 16S rRNA gene of at least about 1, 2, 3, 4, 5, 6, 7, 8, 9, 10, 11, 12, 13, 14, 15 or any range therein, may be used depending upon the bacterium in question. To this end, the gene copy number of the bacterial 16S rRNA gene of a particular bacterium, such as that identified in the aforementioned method, may be found at the Ribosomal RNA Operon Copy Number Database (rrndb: https://rrndb.umms.med.umich.edu/). In other embodiments, the gene copy number utilised may be an average or median number of the gene copy number across a number of species, variants and/or strains of a bacterium. Exemplary gene copy numbers for the 16S rRNA gene for a range of bacterial species are provided in Tables 1 and 15 below.

TABLE 1
Gene copy numbers for the 16S rRNA gene

| | | | 16S rRNA |
|---|---|---|---|
| Bacterial species | | Strain | Copies |
| Staphylococcus | aureus | ATCC 29213 | 5 |
| Staphylococcus | epidermidis | ATCC 14990 | 5-6 |
| Streptococcus | agalactiae | ATCC 13813 | 5-7 |
| Serratia | marcescens | ATCC 8100 | 7 |
| Proteus | mirabilis | ATCC 7002 | 6-7 |
| Streptococcus | pneumoniae | ATCC 6306 | 4 |
| Enterobacter | cloacae | ATCC 13047 | 8 |
| Pseudomonas | aeruginosa | ATCC 27853 | 4 |
| Streptococcus | pyogenes | ATCC 19615 | 5-6 |
| Enterococcus | faecalis | ATCC 29212 | 4 |
| Enterococcus | faecium | ATCC 27270 | 6 |
| Bacteroides | fragilis | ATCC | 6 |
| Escherichia | coli | ATCC 25922 | 7 |
| Klebsiella | pneumoniae | ATCC 13883 | 8 |
| Haemophilus | influenzae | ATCC 9006 | 6 |

Suitably, the method of the aforementioned aspects, may include the further step of identifying the bacterium. It is envisaged that the bacterium may be identified by any means known in the art. Such an identification step may further be performed before, after or simultaneously with one or more of the aforementioned steps of the method for quantifying the bacterium. Preferably, the step of identifying the bacterium comprises analysing the amplification product for the presence or absence of at least one SNP, such as those SNPs described herein.

In one embodiment, said one or plurality of SNPs in the at least a portion of the bacterial 16S rRNA gene is selected from SNPs at positions corresponding to positions 273, 378, 408, 412, 440, 488, 647 and 653 of the 16S rRNA gene as set forth in SEQ ID NO:1. In some embodiments, more than one SNP may be used in the methods of the present invention. For example, at least two SNPs, at least three SNPs, at least four SNPS, at least five SNPs, at least six SNPs, or even at least seven SNPs may be used.

In another embodiment, said one or plurality of SNPs in the at least a portion of the bacterial 16S rRNA gene may be in or correspond to the 16S rRNA gene as set forth in SEQ ID NO: 38. The one or plurality of SNPs in the at least a portion of the bacterial 16S rRNA gene set forth in SEQ ID NO: 38 may be at a position corresponding to at least one of positions 746, 764, 771, or 785 of the 16S rRNA gene set forth in SEQ ID NO: 38 (or positions 737, 755, 762, or 776 of the 16S rRNA gene as set forth in SEQ ID NO:1). At least one said SNP, at least two said SNPs, at least three said SNPs or at least four said SNPs may be used.

Accordingly, in some embodiments said method comprises analysing at least a portion of a bacterial 16S rRNA gene or gene product from the sample, for the presence or absence of:

single nucleotide polymorphisms in the bacterial 16S rRNA gene at a position corresponding to positions 273, 378, 408, 412, 440, 488, 647, and 653 of the 16S rRNA gene set forth in SEQ ID NO: 1; or

single nucleotide polymorphisms in the bacterial 16S rRNA gene a position corresponding to positions 746, 764, 771, and 785 of the 16S rRNA gene set forth in SEQ ID NO: 38.

In a further embodiment, said method comprises analysing at least a portion of a bacterial 16S rRNA gene or gene product from the sample, for the presence or absence of:

single nucleotide polymorphisms in the at least a portion of the bacterial 16S rRNA gene or gene product at a position corresponding to at least four of positions 273, 378, 408, 412, 440, 488, 647, 653, 737, 755, 762 and 776 of the 16S rRNA gene set forth in SEQ ID NO: 1.

In some embodiments, the bacterium or bacteria is or are selected from among mammalian (e.g., human) associated bacteria, soil associated bacteria and water associated bacteria. In particular embodiments, the bacterium or bacteria may be a sepsis-associated bacterium or bacteria.

In one embodiment, the bacterium or bacteria is or are selected from a gram-negative bacterium or gram-negative bacteria. In one embodiment, the bacterium or bacteria is or are selected from a gram-positive bacterium or gram-positive bacteria. In one embodiment, the bacterium or bacteria is or are selected from a bacterium or bacteria from the firmicutes phylum. In one embodiment, the bacterium or bacteria is or are selected from a bacterium or bacteria from the actinobacteria phylum. In one embodiment, the bacterium or bacteria is or are selected a bacterium or bacteria from the proteobacteria phylum.

In some particular embodiments, the bacterium or bacteria is or are selected from among at least one of: Acinetobacter spp.; Actinobaccillus spp.; Actinomadura spp.; Actinomyces spp.; Actinoplanes spp.; Aerococcus spp.; Aeromonas spp.; Agrobacterium spp.; Alistipes spp.; Anaerococcus spp.; Arthrobacter spp.; Bacillus spp.; Bacteroides spp.; Brucella spp.; Bulleidia spp.; Burkholderia spp.; Cardiobacterium spp.; Cedecea spp.; Citrobacter spp.; Clostridium spp.; Cornyebacterium spp.; Cronobacter spp.; Dermatophilus spp.; Dorea spp; Enterobacter spp.; Enterococcus spp.; Erysipelothrix spp.; Escherichia spp.; Eubacterium spp.; Ewardsiella spp.; Faecalibacterium spp.; Filifactor spp.; Finegoldia spp.; Flavobacterium spp.; Francisella spp.; Gallicola spp.; Haemophilus spp.; Helococcus spp.; Holdemania spp.; Hyphomicrobium spp.; Klebsiella spp.; Lactobacillus spp.; Legionella spp.; Listeria spp.; Methylobacterium spp.; Micrococcus spp.; Micromonospora spp.; Mobiluncus spp.; Moraxella spp.; Morganella spp.; Mycobacterium spp.; Neisseria spp.; Nocardia spp.; Paenibacillus spp.; Parabacteroides spp.; Pasteurella spp.; Peptoniphilus spp.; Peptostreptococcus spp.; Planococcus spp.; Planomicrobium spp.; Plesiomonas spp.; Porphyromonas spp.; Prevotella spp.; Propionibacterium spp.; Proteus spp.; Providentia spp.; Pseudomonas spp.; Ralstonia spp.; Rhodococcus spp.; Roseburia spp.; Ruminococcus spp.; Salmonella spp.; Sedimentibacter spp.; Serratia spp.; Shigella spp.; Shewanella spp.; Solobacterium spp.; Sphingomonas spp.; Staphylococcus spp.; Stenotrophomonas spp.; Streptococcus spp.; Streptomyces spp.; Tissierella spp.; Vibrio spp.; and Yersinia spp.

In some more particular embodiments, the bacterium or bacteria is or are selected from among at least one of: Acinetobacter baumannii; Acinetobacter calcoaceticus; Aerococcus viridans; Bacteroides fragilis; Bacteroides vulgatus; Cedecea lapagei; Citrobacter freundii; Cronobacter dublinensis; Enterobacter aerogenes; Enterobacter cloacae; Enterococcus avium; Enterococcus cecorum; Enterococcus faecalis; Enterococcus faecium; Escherichia coli; Haemophilus influenzae; Klebsiella oxytoca; Klebsiella pneumoniae; Morganella morganii; Proteus mirabilis; Pseudomonas aeruginosa; Serratia marcescens; Shewanella putrefaciens; Staphylococcus aureus; Staphylococcus epidermidis; Staphylococcus hominis; Staphylococcus saprophyticus; Stenotrophomonas maltophilia; Streptococcus agalactiae; Streptococcus anginosus; Streptococcus constellatus; Streptococcus intermedius; Streptococcus milleri; Streptococcus mitis; Streptococcus mutans; Streptococcus oralis; Streptococcus pneumoniae; Streptococcus pyogenes; Streptococcus sanguinis; and Streptococcus sobrinus.

In particular embodiments, the bacterium or bacteria is or are selected from among at least one of: Acinetobacter calcoaceticus; Enterobacter aerogenes; Enterobacter cloacae; Enterococcus faecalis; Enterococcus faecium; Escherichia coli; Klebsiella pneumoniae; Proteus mirabilis; Pseudomonas aeruginosa; Serratia marcescens; Staphylococcus aureus; Staphylococcus epidermidis; Streptococcus agalactiae; Streptococcus pneumoniae; and Streptococcus pyogenes.

In particular embodiments, the bacterium or bacteria is or are selected from among at least one of: Acinetobacter calcoaceticus; Enterobacter aerogenes; Enterobacter cloacae; Enterococcus faecalis; Enterococcus faecium; Escherichia coli; Klebsiella pneumoniae; Proteus mirabilis; Pseudomonas aeruginosa; Serratia marcescens; Staphylococcus aureus; Staphylococcus epidermidis; Streptococcus agalactiae; Streptococcus pneumoniae; Streptococcus pyogenes; Listeria monocytogenes; Clostridium perfringens; Corynebacterium jeikeium; Bacteroides fragilis; Neisseria meningitides; Haemophilus influenzae; Salmonella sp.; and Staphylococcus epidermidis. In another embodiment, the bacterium or bacteria is or are selected from among at least one of: Acinetobacter calcoaceticus; Enterobacter aerogenes; Enterobacter cloacae; Enterococcus faecalis; Enterococcus faecium; Escherichia coli; Klebsiella pneumoniae; Proteus mirabilis; Pseudomonas aeruginosa; Serratia marcescens; Staphylococcus aureus; Staphylococcus epidermidis; Streptococcus agalactiae; Streptococcus pneumoniae; Streptococcus pyogenes; Listeria monocytogenes; Clostridium perfringens; Corynebacterium jeikeium; Bacteroides fragilis; Neisseria meningitides; Haemophilus influenzae; Salmonella sp.; Staphylococcus epidermidis; Bacillus anthracis, Clostridium botulinum, Yersinia pestis, Francisella tularensis, Vibrio cholerae, and Burkholderia pseudomallei.

In one embodiment the bacterium or bacteria is a Security Sensitive Biological Agent (SSBA). The SSBA may be a Tier 1 agent or a Tier 2 agent. Exemplary Tier 1 agents include one or more of: Bacillus anthracis (Anthrax), and Yesinia pestis (Plague). Exemplary Tier 2 agents include one or more of: Clostridium botulinum (botulism, especially toxin producing strains); Francisella tularensis (Tularaemia); Salmonella Typhi (typhoid), and Vibrio cholerae (especially Cholera serotypes O1 or O139). In one embodiment the bacterium or bacteria is or are selected from among at least one of the group consisting of: Bacillus anthracis, Clostridium botulinum, Yersinia pestis, Francisella tularensis, Vibrio cholerae, and Burkholderia pseudomallei.

In one embodiment, the bacterium is a human pathogen.

In some embodiments, the methods of the present invention may be used to analyse blood from a subject with systemic inflammatory response syndrome (SIRS) to determine the origin of the SIRS (for example bacteria). In other embodiments, the methods of the present invention may be used to determine whether a subject has sepsis having a microbial infectious origin. In both embodiments, the methods of the present invention may be used to determine the presence of, differentiate and/or identify microorganisms, such as bacteria, present in the sample.

SIRS is an overwhelming whole body reaction that may have an infectious aetiology or non-infectious aetiology (i.e., infection-negative SIRS, or inSIRS). Sepsis is SIRS that occurs during infection. Sepsis in this instance is diagnosed by a clinician (when there is suspected infection) or through culture of an organism. Both SIRS and sepsis are defined by a number of non-specific host response parameters including changes in heart and respiratory rate, body temperature and white cell counts (Levy et al., 2003; Reinhart et al., 2012).

In some embodiments, the at least one SNP or at least one probe, tool or reagent may be used to classify bacteria in a sample as Gram-positive bacterium or bacteria or Gram-negative bacterium or bacteria.

For example, in some embodiments, the bacterium may be classified as Gram-positive bacteria based on any one of the above SNPs, especially at at least one of positions 273, 378, 408, 412, 440, 488, 647, and 653 of the 16S rRNA gene set forth in SEQ ID NO: 1. In one such embodiment, the bacterium may be classified based on SNPs at positions corresponding to positions 273 and 653 of the 16S rRNA gene as set forth in SEQ ID NO:1, wherein the bacterium is determined to be Gram-positive when there is an A at position 273 and a T at position 653.

For example, in another embodiment, the bacterium may be classified as Gram-positive based on at least one SNP at a position corresponding to position 440 of the 16S rRNA gene as set forth in SEQ ID NO:1, wherein the bacterium is determined to be Gram-positive when there is a T at position 440. Conversely, wherein the bacterium is determined to be Gram-negative when there is not a T at position 440.

In yet other embodiments, the at least one SNP or at least one probe, tool or reagent may be used to classify groups of microorganisms, and in particular bacteria, in a sample.

For example, in some embodiments, the bacteria may be classified as belonging to a particular genus based on at least one SNP selected from the above SNPs. In one such embodiment, the bacteria may be classified as belonging to a particular genus based on at least one SNP selected from SNPs at positions corresponding to positions 412 and 647 of the 16S rRNA gene as set forth in SEQ ID NO:1. For example, the bacterium or bacteria in a sample may be classified as belonging to the Staphylococcus genus when there is a T at position 412. For example, the bacterium or bacteria in a sample may be classified as belonging to the Enterococcus genus when there is a G at position 647.

In yet other embodiments, the at least one SNP or at least one probe, tool or reagent may be used to identify a bacterium in a sample as described above.

For example, the bacterium Enterobacter cloacae may be identified in a sample based on at least one SNP at a position corresponding to position 653 of the 16S rRNA gene as set forth in SEQ ID NO:1, wherein the bacterium Enterobacter cloacae is identified when there is a G at position 653.

For example, bacterium selected from Streptococcus pneumoniae, Streptococcus agalactiae and Streptococcus pyogenes may be identified in a sample based on SNPs at positions corresponding to positions 378 and 488 of the 16S rRNA gene as set forth in SEQ ID NO:1, wherein the bacterium is: Streptococcus pneumoniae when there is an A at position 378 and a T at position 488; Streptococcus agalactiae when there is an A at position 378 and an A 488; and Streptococcus pyogenes when there is a G at position 378 and an A at position 488.

For example, in one embodiment, bacterium selected from among Acinetobacter calcoaceticus; Enterobacter cloacae; Escherichia coli; Klebsiella pneumoniae; Proteus mirabilis; Pseudomonas aeruginosa; Streptococcus agalactiae; Streptococcus pneumoniae; and Streptococcus pyogenes may be identified in a sample based on SNPs at positions corresponding to positions 273, 378, 408, 412, 440, 488, 647 and 653 of the 16S rRNA gene as set forth in SEQ ID NO:1, wherein the bacterium is: Acinetobacter calcoaceticus when there is an A at positions 273, 440 and 647; Enterobacter cloacae when there is a G at position 653; Escherichia coli when there is a T at position 273 and a T at position 653; Klebsiella pneumoniae when there is a T at position 273, a C at positions 488 and 647 and an A at position 653; Proteus mirabilis when there is a C at positions 440 and 488 and a T at position 647; Pseudomonas aeruginosa when there is an A at position 440 and a T at position 647; Streptococcus agalactiae when there is an A at positions 378, 488 and 647; Streptococcus pneumoniae when there is Tat positions 488 and 647; and Streptococcus pyogenes when there is G at position 378 and A at positions 488 and 647.

For example, in another embodiment, bacterium selected from among Acinetobacter calcoaceticus; Enterobacter cloacae; Escherichia coli; Klebsiella pneumoniae; Proteus mirabilis; Pseudomonas aeruginosa; Streptococcus agalactiae; Streptococcus pneumoniae; and Streptococcus pyogenes may be identified in a sample based on the presence of SNPs set forth in the Table 2:

TABLE 2

| | | SNP position in the 16S rRNA gene | | | | | | |
|---|---|---|---|---|---|---|---|---|
| | | as set forth in SEQ ID NO: 1 | | | | | | |
| Bacterial species | 273 | 378 | 408 | 412 | 440 | 488 | 647 | 653 |
| Escherichia coli | T | G | A | A | C | C | C | T |
| Streptococcus | A | A | G | A | T | T | T | T |
| pneumoniae | | | | | | | | |
| Streptococcus | A | A | G | A | T | A | A | T |
| agalactiae | | | | | | | | |
| Streptococcus | A | G | G | A | T | A | A | T |
| pyogenes | | | | | | | | |
| Proteus mirabilis | T | G | G | A | C | C | T | A |
| Enterobacter | T | G | A | A | C | C | C | G |
| cloacae | | | | | | | | |
| Klebsiella | T | G | G | A | C | C | C | A |
| pneumoniae | | | | | | | | |
| Pseudomonas | A | G | G | A | A | C | T | A |
| aeruginosa | | | | | | | | |
| Acinetobacter | A | G | G | A | A | C | A | A |
| calcoaceticus | | | | | | | | |

For example, in another embodiment, bacterium selected from among Escherichia coli, Streptococcus pneumoniae, Streptococcus agalactiae, Streptococcus pyogenes, Proteus mirabilis, Enterobacter cloacae, Klebsiella pneumoniae, Pseudomonas aeruginosa, Acinetobacter calcoaceticus, Enterococcus faecalis, Listeria monocytogenes, Staphylococcus aureus, Clostridium perfringens, Corynebacterium jeikeium, Bacteroides fragilis, Neisseria meningitidis, Haemophilus influenzae, Serratia marcescens, Salmonella sp., and Staphylococcus epidermidis may be identified in a sample based on the presence of SNPs set forth in Table 3.

In another embodiment, bacterium selected from among Bacillus anthracis, Clostridium botulinum type A, Clostridium botulinum type B, Clostridium botulinum type C, Clostridium botulinum type D, Clostridium botulinum type G, Yersinia pestis, Francisella tularensis, Vibrio cholerae and Burkholderia pseudomallei may be identified in a sample based on SNPs at positions corresponding to positions 746, 764, 771, or 785 of the 16S rRNA gene as set forth in SEQ ID NO:38, wherein the bacterium is: Bacillus anthracis when there is a T at position 746, A at position 764, C at position 771 and G at position 785; Clostridium botulinum type A or Clostridium botulinum type B when there is a T at position 746, G at position 764, C at position 771 and T at position 785; Clostridium botulinum type C when there is a T at position 746, A at position 764, T at position 771 and T at position 785; Clostridium botulinum type D when there is a C at position 746, A at position 764, T at position 771 and T at position 785; Clostridium botulinum type G when there is a T at position 746, G at position 764, C at position 771 and G at position 785; Yersinia pestis when there is a C at position 746, G at position 764, T at position 771 and G at position 785; Francisella tularensis when there is a T at position 746, A at position 764, G at position 771 and G at position 785; Vibrio cholerae when there is a C at position 746, A at position 764, T at position 771 and G at position 785; and Burkholderia pseudomallei when there is a C at position 746, G at position 764, C at position 771 and G at position 785.

For example, in another embodiment, bacterium selected from among Bacillus anthracis, Clostridium botulinum type A, Clostridium botulinum type B, Clostridium botulinum type C, Clostridium botulinum type D, Clostridium botulinum type G, Yersinia pestis, Francisella tularensis, Vibrio cholerae and Burkholderia pseudomallei may be identified in a sample based on the presence of SNPs set forth in Table 4.

TABLE 3

| | | SNP position in the 16S rRNA gene as set fort h in SEQ ID NO: 1 | | | | | | | | | | |
|---|---|---|---|---|---|---|---|---|---|---|---|---|
| Bacterial species | 273 | 378 | 408 | 412 | 440 | 488 | 647 | 653 | 737 | 755 | 762 | 776 |
| Escherichia coli | T | G | A | A | C | C | C | T | C | G | T | G |
| Streptococcus pneumoniae | A | A | G | A | T | T | T | T | C | G | C | G |
| Streptococcus agalactiae | A | A | G | A | T | A | A | T | C | G | C | G |
| Streptococcus pyogenes | A | G | G | A | T | A | A | T | C | G | T | G |
| Proteus mirabilis | T | G | G | A | C | C | T | A | C | G | T | G |
| Enterobacter cloacae | T | G | A | A | C | C | C | G | C | G | T | G |
| Klebsiella pneumoniae | T | G | G | A | C | C | C | A | C | G | T | G |
| Pseudomonas aeruginosa | A | G | G | A | A | C | T | A | A | A | T | G |
| Acinetobacter calcoaceticus | A | G | G | A | A | C | A | A | A | G | T | A |
| Enterococcus faecalis | A | A | G | A | T | T | G | T | C | G | C | G |
| Listeria monocytogenes | A | A | A | A | T | T | A | G | C | G | T | G |
| Staphylococcus aureus | A | G | G | T | T | C | A | T | T | G | T | G |
| Clostridium perfringens | A | G | G | T | C | C | T | A | C | G | C | G |
| Corynebacterium jeikeium | A | G | G | T | C | C | C | A | C | G | A | G |
| Bacteroides fragilis | A | G | A | T | T | A | C | T | C | A | C | T |
| Neisseria meningitidis | A | G | C | A | T | T | G | C | T | G | C | T |
| Haemophilus influenzae | A | G | A | A | C | C | G | A | C | G | C | G |
| Serratia marcescens | T | G | G | A | C | C | C | A | C | G | T | G |
| Salmonella sp. | A | G | A | A | C | C | C | T | C | G | T | G |
| Staphylococcus epidermidis | C | G | T | A | C | T | C | T | T | G | T | G |

TABLE 4

| | SNP position in the 16S rRNA gene | | | |
|---|---|---|---|---|
| | as set forth in SEQ ID NO: 38 | | | |
| Bacterial species | 746 | 764 | 771 | 785 |
| Bacillus anthracis | T | A | C | G |
| Clostridium botulinum type A | T | G | C | T |
| Clostridium botulinum type B | T | G | C | T |
| Clostridium botulinum type C | T | A | T | T |
| Clostridium botulinum type D | C | A | T | T |
| Clostridium botulinum type G | T | G | C | G |
| Yersinia pestis | C | G | T | G |
| Francisella tularensis | T | A | G | G |
| Vibrio cholerae | C | A | T | G |
| Burkholderia pseudomallei | C | G | C | G |

The cumulative discrimatory index of the four SNPs used to identify the above organisms are 0.667 for 1 SNP; 0.889 for 2 SNPs; 0.944 for 3 SNPs; and 0.972 for 4 SNPs.

Position 746 of the 16S rRNA gene set forth in SEQ ID NO:38 corresponds to position 737 of the 16S rRNA gene set forth in SEQ ID NO:1. Position 764 of the 16S rRNA gene set forth in SEQ ID NO:38 corresponds to position 755 of the 16S rRNA gene set forth in SEQ ID NO:1. Position 771 of the 16S rRNA gene set forth in SEQ ID NO:38 corresponds to position 762 of the 16S rRNA gene set forth in SEQ ID NO:1. Position 785 of the 16S rRNA gene set forth in SEQ ID NO:38 corresponds to position 776 of the 16S rRNA gene set forth in SEQ ID NO:1.

Therefore, in another embodiment, bacterium selected from among Bacillus anthracis, Clostridium botulinum type A, Clostridium botulinum type B, Clostridium botulinum type C, Clostridium botulinum type D, Clostridium botulinum type G, Yersinia pestis, Francisella tularensis, Vibrio cholerae and Burkholderia pseudomallei may be identified in a sample based on SNPs at positions corresponding to positions 737, 755, 762, or 776 of the 16S rRNA gene as set forth in SEQ ID NO:1, wherein the bacterium is: Bacillus anthracis when there is a T at position 737, A at position 755, C at position 762 and G at position 776; Clostridium botulinum type A or Clostridium botulinum type B when there is a T at position 737, G at position 755, C at position 762 and T at position 776; Clostridium botulinum type C when there is a T at position 737, A at position 755, T at position 762 and T at position 776; Clostridium botulinum type D when there is a C at position 737, A at position 755, T at position 762 and T at position 776; Clostridium botulinum type G when there is a T at position 737, G at position 755, C at position 762 and G at position 776; Yersinia pestis when there is a C at position 737, G at position 755, T at position 762 and G at position 776; Francisella tularensis when there is a T at position 737, A at position 755, G at position 762 and G at position 776; Vibrio cholerae when there is a C at position 737, A at position 755, T at position 762 and G at position 776; and Burkholderia pseudomallei when there is a C at position 737, G at position 755, C at position 762 and G at position 776.

The bacterium may be partially identified or classified based on one or more of the above SNPs.

The SNPs may be analysed by any method known in the art including, but not limited to: high resolution melt analysis, 5′ nuclease digestion (including 5′ nuclease digestion), molecular beacons, oligonucleotide ligation, microarray, restriction fragment length polymorphism; antibody detection methods; direct sequencing or any combination thereof. In one embodiment, the step of analysing in the methods comprises determining the presence or the absence of the at least one SNP using high resolution melt analysis, 5′ nuclease digestion, molecular beacons, oligonucleotide ligation, microarray, restriction fragment length polymorphism, antibody detection methods; direct sequencing or any combination thereof. The SNPs may be detected by any method known in the art including, but not limited to: polymerase chain reaction (PCR); ligase chain reaction (LCR); hybridization analysis; high-resolution melt analysis; digestion with nucleases, including 5′ nuclease digestion; molecular beacons; oligonucleotide ligations; microarray; restriction fragment length polymorphism; antibody detection methods; direct sequencing; or any combination thereof.

For example, in some embodiments, identifying or classifying the bacteria may further be based on DNA melting characteristics of the SNPs as broadly described above and their surrounding DNA sequences, preferably high-resolution melt analysis, such as described in PCT/AU2018/050471, which is incorporated by reference herein.

For example, in some such embodiments, the methods of the present invention may further include high-resolution melt (HRM) analysis to further analyse the DNA melting characteristics of the SNPs as broadly described above and their surrounding DNA sequences. In a particular embodiment, the HRM analysis may include forming a DNA amplification product (i.e., amplicon) containing at least one of the SNPs and at least one intercalating fluorescent dye and heating the DNA amplification product through its melting temperature (Tₘ). The HRM is monitored in real-time using the fluorescent dye incorporated into the DNA amplification product. The level of fluorescence is monitored as the temperature increases with the the fluorescence reducing as the amount of double-stranded DNA reduces. Changes in fluorescence and temperature can be plotted in a graph known as a melt curve.

As a skilled addressee will understand, the Tₘ of the DNA amplification product at which the two DNA strands separate is predictable, being dependent on the sequence of the nucleotide bases forming the DNA amplification product. Accordingly, it is possible to differentiate between DNA amplification products including a DNA amplification product containing a polymorphism (i.e., a SNP or SNPs) as the melt curves will appear different. Indeed, in some embodiments, it is possible to differentiate between DNA amplification products containing the same polymorphism based on differences in the surrounding DNA sequences.

For example, bacterium selected from among Acinetobacter calcoaceticus; Enterobacter aerogenes; Enterobacter cloacae; Enterococcus faecalis; Enterococcus faecium; Escherichia coli; Klebsiella pneumoniae; Proteus mirabilis; Pseudomonas aeruginosa; Serratia marcescens; Staphylococcus aureus; Staphylococcus epidermidis; Streptococcus agalactiae; Streptococcus pneumoniae; and Streptococcus pyogenes may be identified in a sample based on the presence of SNPs set forth in the Table 5 and DNA melting characteristics of the SNPs and their surrounding DNA sequences:

TABLE 5

| | | SNP position in the 16S rRNA gene | | | | | | |
|---|---|---|---|---|---|---|---|---|
| | | as set forth in SEQ ID NO: 1 | | | | | | |
| Bacterial species | 273 | 378 | 408 | 412 | 440 | 488 | 647 | 653 |
| Escherichia coli | T | G | A | A | C | C | C | T |
| Staphylococcus | A | G | G | T | T | C | A | T |
| aureus | | | | | | | | |
| Staphylococcus | A | G | T | T | T | C | A | T |
| epidermidis | | | | | | | | |
| Streptococcus | A | A | G | A | T | T | T | T |
| pneumoniae | | | | | | | | |
| Streptococcus | A | A | G | A | T | A | A | T |
| agalactiae | | | | | | | | |
| Streptococcus | A | G | G | A | T | A | A | T |
| pyogenes | | | | | | | | |
| Enterococcus | A | A | G | A | T | T | G | T |
| faecalis | | | | | | | | |
| Enterococcus | A | A | G | A | T | T | G | T |
| faecium | | | | | | | | |
| Proteus mirabilis | T | G | G | A | C | C | T | A |
| Serratia marcescens | T | G | G | A | C | T | C | A |
| Enterobacter | T | G | A | A | C | T | C | A |
| aerogenes | | | | | | | | |
| Enterobacter | T | G | A | A | C | C | C | G |
| cloacae | | | | | | | | |
| Klebsiella | T | G | G | A | C | C | C | A |
| pneumoniae | | | | | | | | |
| Pseudomonas | A | G | G | A | A | C | T | A |
| aeruginosa | | | | | | | | |
| Acinetobacter | A | G | G | A | A | C | A | A |
| calcoaceticus | | | | | | | | |

For example, bacterium selected from among Escherichia coli, Streptococcus pneumoniae, Streptococcus agalactiae, Streptococcus pyogenes, Proteus mirabilis, Enterobacter cloacae, Klebsiella pneumoniae, Pseudomonas aeruginosa, Acinetobacter calcoaceticus, Enterococcus faecalis, Listeria monocytogenes, Staphylococcus aureus, Clostridium perfringens, Corynebacterium jeikeium, Bacteroides fragilis, Neisseria meningitidis, Haemophilus influenzae, Serratia marcescens, Salmonella sp., Staphylococcus epidermidis may be identified in a sample based on the presence of SNPs set forth in Table 3 and DNA melting characteristics of the SNPs and their surrounding DNA sequences.

In one embodiment, bacteria selected from Acinetobacter calcoaceticus; Enterobacter aerogenes; Enterobacter cloacae; Enterococcus faecalis; Enterococcus faecium; Escherichia coli; Klebsiella pneumoniae; Proteus mirabilis; Pseudomonas aeruginosa; Serratia marcescens; Staphylococcus aureus; Staphylococcus epidermidis; Streptococcus agalactiae; Streptococcus pneumoniae; and Streptococcus pyogenes may be identified in a sample based on SNPs at positions corresponding to positions 273, 378, 408, 412, 440, 488, 647 and 653 of the 16S rRNA gene as set forth in SEQ ID NO:1 and high-resolution melt curve analysis of the SNPs and their surrounding DNA.

For example, the bacterium selected from among Acinetobacter calcoaceticus; Enterobacter cloacae; Escherichia coli; Klebsiella pneumoniae; Proteus mirabilis; Pseudomonas aeruginosa; Streptococcus agalactiae; Streptococcus pneumoniae; and Streptococcus pyogenes may be identified in the sample based on the SNP positions as described above and/or high-resolution melt curve analysis of the SNPs and their surrounding DNA.

In some embodiments. the bacterium selected from Staphylococcus aureus; Staphylococcus epidermidis; Enterococcus faecalis; Enterococcus faecium, Serratia marcescens; and Enterobacter aerogenes may be individually identified in a sample based on SNPs at positions corresponding to positions 412, 440, 488 and 647 of the 16S rRNA gene as set forth in SEQ ID NO:1, wherein: Staphylococcus aureus and Staphylococcus epidermidis may be identified when there is a T at position 412 and then further distinguished from one another based on high-resolution melt curve analysis of the DNA surrounding the SNP at position 412; Enterococcus faecalis and Enterococcus faecium may be identified when there is a G at position 647 and then further distinguished from one another based on high-resolution melt curve analysis of the DNA surrounding the SNP at position 647; Serratia marcescens and Enterobacter aero genes may be identified when there is a C at positions 440 and 647 and a T at position 488 and may then be further distinguished from one another based on high-resolution melt curve analysis of the DNA surrounding the SNPs at any one of positions 440, 488 and 647.

In other embodiments, the bacterium selected from Enterococcus faecalis; Enterococcus faecium, Streptococcus agalactiae; and Streptococcus pyogenes may be identified in a sample based on at least one SNP at a position corresponding to position 378 of the 16S rRNA gene as set forth in SEQ ID NO:1 and high-resolution melt curve analysis of the DNA surrounding the SNP at position 378.

For example, bacterium selected from among Bacillus anthracis, Clostridium botulinum type A, Clostridium botulinum type B, Clostridium botulinum type C, Clostridium botulinum type D, Clostridium botulinum type G, Yersinia pestis, Francisella tularensis, Vibrio cholerae and Burkholderia pseudomallei may be identified in a sample based on the presence of SNPs set forth in Table 6 and DNA melting characteristics of the SNPs and their surrounding DNA sequences:

TABLE 6

| | SNP position in the 16S rRNA gene | | | |
|---|---|---|---|---|
| | as set forth in SEQ ID NO: 38 | | | |
| Bacterial species | 746 | 764 | 771 | 785 |
| Bacillus anthracis | T | A | C | G |
| Clostridium botulinum type A | T | G | C | T |
| Clostridium botulinum type B | T | G | C | T |
| Clostridium botulinum type C | T | A | T | T |
| Clostridium botulinum type D | C | A | T | T |
| Clostridium botulinum type G | T | G | C | G |
| Yersinia pestis | C | G | T | G |
| Francisella tularensis | T | A | G | G |
| Vibrio cholerae | C | A | T | G |
| Burkholderia pseudomallei | C | G | C | G |

As noted above, position 746 of the 16S rRNA gene set forth in SEQ ID NO:38 corresponds to position 737 of the 16S rRNA gene set forth in SEQ ID NO:1. Position 764 of the 16S rRNA gene set forth in SEQ ID NO:38 corresponds to position 755 of the 16S rRNA gene set forth in SEQ ID NO:1. Position 771 of the 16S rRNA gene set forth in SEQ ID NO:38 corresponds to position 762 of the 16S rRNA gene set forth in SEQ ID NO:1. Position 785 of the 16S rRNA gene set forth in SEQ ID NO:38 corresponds to position 776 of the 16S rRNA gene set forth in SEQ ID NO:1.

In one embodiment, bacteria selected from Bacillus anthracis, Clostridium botulinum type A, Clostridium botulinum type B, Clostridium botulinum type C, Clostridium botulinum type D, Clostridium botulinum type G, Yersinia pestis, Francisella tularensis, Vibrio cholerae and Burkholderia pseudomallei may be identified in a sample based on SNPs at positions corresponding to positions 746, 764, 771, or 785 of the 16S rRNA gene as set forth in SEQ ID NO:38 (or positions 737, 755, 762, or 776 of the 16S rRNA gene as set forth in SEQ ID NO:1) and high-resolution melt curve analysis of the SNPs and their surrounding DNA.

For example, the bacterium selected from among Bacillus anthracis, Clostridium botulinum type A, Clostridium botulinum type B, Clostridium botulinum type C, Clostridium botulinum type D, Clostridium botulinum type G, Yersinia pestis, Francisella tularensis, Vibrio cholerae and Burkholderia pseudomallei may be identified in the sample based on the SNP positions as described above and/or high-resolution melt curve analysis of the SNPs and their surrounding DNA.

In some embodiments, the methods of the invention may further include administering a therapeutic agent to the subject, such as, e.g., an antibiotic or antimicrobial agent. In another embodiment, a method of evaluating treatment efficacy (for example as in the fourth aspect of the present invention) may further comprise the step of determining whether the at least one bacteria is at least partly sensitive and/or resistant to a therapeutic agent.

In one embodiment, the methods described herein further include the step selecting a treatment for the infection based on the quantity or concentration of the bacterium in the sample or the biological sample.

It will be appreciated that the method of treating the infection may include administration of a therapeutically effective amount of one or more therapeutic agents that facilitate treatment thereof. By way of example only, these may include: antibiotic agents (inclusive of small molecule antibiotics, molecules that are antimicrobial in nature, natural or synthetic peptide antimicrobials and/or proteins with antimicrobial properties), anti-inflammatory agents (e.g., non-steroidal anti-inflammatory agents (NSAIDs), corticosteroids), immunosuppressant agents, immunomodulatory agents, oxygen, intravenous fluids and vasopressor agents.

Exemplary antibiotic agents include fluoroquinolones (including ciprofloxacin), tetracyclines (including doxycycline), macrolides (including erythromycin, cethromycin, azithromycin and clarithromycin), 3-lactams (including penicillin, imipenem and ampicillin), ansamycins (including rifampin), phenicols (including chloramphenicol), streptogramins (including quinupristin-dalfopristin), aminoglycosides (including gentamicin), oxazolidinones (including linezolid), tetracyclines, glycylglycines (including tigecycline), cyclic lipopeptides (including daptomycin) and lincosamines (including clindamycin). Specific examples of other antibiotic agents include fusidic acid, trimethoprim, sulfadiazine, sulfamethoxazole, a penicillin, a monobactam, a penam, a penem, a clavam, a clavem, a carbopenam, a carbopenem, a cepham, a cephem, an oxacepham, an oxacephem, a carbocepham, a carbocephem, a cephalosporin, tetracycline, a tetracycline derived antibacterial agent, glycylcycline, a glycylcycline derived antibacterial agent, minocycline, a minocycline derived antibacterial agent, sancycline, a sancycline derived antibacterial agent, methacycline, a methacycline derived antibacterial agent, an oxazolidinone antibacterial agent, an aminoglycoside antibacterial agent, a quinolone antibacterial agent, daptomycin, a daptomycin derived antibacterial agent, rifamycin, a rifamycin derived antibacterial agent, rifampin, a rifampin derived antibacterial agent, rifalazil, a rifalazil derived antibacterial agent, rifabutin, a rifabutin derived antibacterial agent, rifapentin, a rifapentin derived antibacterial agent, rifaximin and a rifaximin derived antibacterial agent.

As will be appreciated, the quantity or concentration of the bacterium in the biological sample will typically increase with disease progression.

In this regard, an increase of the quantity or concentration of the bacterium in the biological sample of a subject undergoing treatment, may indicate disease progression in the subject, and that the treatment is inefficacious (e.g., drug resistance), whilst a decrease in the quantity or concentration of the bacterium in the biological sample of a subject undergoing treatment generally indicates disease remission or regression in the subject, and therefore that the treatment is efficacious.

In some embodiments, multiple time points prior to, during and/or after treatment of a subject with the infection may be selected to determine the quantity or concentration of the bacterium in a biological sample taken from the subject at these multiple time points to determine a prognosis or treatment efficacy. For example, the quantity or concentration of the bacterium can be determined at an initial time point and then again at one, two, three or more subsequent time points.

Suitably, the time points for taking a biological sample may be selected throughout a treatment cycle or over a desired time period. Over a desired time period, for example, the time points may be prior to treatment, mid way through treatment and/or after treatment has been completed. Suitably, an altered or modulated quantity or concentration level of the bacterium in a biological sample, such as a decrease or reduction, may be utilised by the methods of the invention from the first to second and/or third time points may provide a positive prognosis for a subject with a bacterial infection. Alternatively, an altered or modulated quantity or concentration level of the bacterium in a biological sample, such as an increase therein, utilised by the methods of the invention from the first to second and/or third time points may provide a poor prognosis for a subject with a bacterial infection.

As would be appreciated by the skilled artisan, the quantity or concentration of the bacterium in the aforementioned biological samples may also be related to the severity, stage, recurrence or progression of the infection and/or the efficacy of the treatment.

In one embodiment, biological samples may be sourced and/or collected from a subject at diagnosis and then prior to each cycle of treatment. Suitably, there may be any number of treatment cycles, depending on the subject and the nature and/or stage of the infection, including but not limited to 1, 2, 3, 4, 5, 6, 7, 8, 9, 10, 15 and/or 20 cycles. The treatment cycles may be close together, spread out over a period of time and/or intense cycles at defined time points over a period of time, or any combination of the above.

In further embodiments, samples may be taken both during treatment and/or after treatment has been completed. Suitably, samples may be sourced from a subject at any time point after treatment has been completed, examples of which include 1, 2, 3, 4, 5, 10, 15, 20, 25 and/or 30 days post treatment, 1, 2 and/or 3 weeks post treatment and/or 1, 3, 6 and/or 9 months post treatment and/or 1, 2, 3, 4, 5, 10, 15, 20 and/or 30 years post treatment. The treatment may be completed once the subject is in remission or after at least one or more treatment cycles, depending on the subject and the infection.

In one embodiment, any suitable sample, such as environmental samples and biological samples, may be used in the methods of the present invention. Exemplary biological samples may comprise sputum, saliva, blood, cerebrospinal fluid or urine samples. As used herein, the term “blood” encompasses whole blood or any fractions of blood, such as serum and plasma as conventionally defined.

According to some embodiments, an internal or external standard for quantifying the amplification product may be used.

The probe, tool or reagent may be, but is not limited to, an oligonucleotide, a primer, a nucleic acid, a polynucleotide, DNA, cDNA, RNA, a peptide or a polypeptide. These may be, for example, single stranded or double stranded, naturally occurring, isolated, purified, chemically modified, recombinant or synthetic.

The probe, tool or reagent may be, but is not limited to, an antibody or other type of molecule or chemical entity capable of specifically binding, detecting or identifying at least a portion of a 16S rRNA gene in a sample containing at least one SNP.

The probe, tool or reagent may be any number or combination of the above, and the number and combination will depend on a desired result to be achieved—e.g., detection of SNP at a genomic level (genotyping) or at the RNA transcription level.

The probe, tool or reagent may be isolated. The probe, tool or reagent may be detectably labelled. A detectable label may be included in an amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

In particular embodiments, the at least one probe, tool or reagent is for specifically binding, detecting or identifying of a SNP at the genomic level or transcription level, preferably the former.

In preferred embodiments, the at least one probe, tool or reagent is for specifically binding, detecting or identifying at least a portion of a 16S rRNA gene in a sample containing at least one SNP as described herein.

For the present methods, a single probe (especially primer) may be used with each sample and/or control sample, or multiple probes (especially primers) may be used with each sample and/or control sample (i.e. in one pot). Such probes (especially primers) can be added to the raw solution obtained from amplification (such as PCR).

In one embodiment, the at least one probe, tool or reagent may comprise two primers, each of which hybridizes to at least a portion of a bacterial 16S rRNA gene (or gene product), containing a SNP as defined above.

In one embodiment, said at least one probe, tool or reagent comprises an oligonucleotide having (or comprising or consisting of) a nucleotide sequence having at least 90%, at least 91%, at least 92%, at least 93%, at least 94%, at least 95%, at least 96%, at least 97%, at least 98%, at least 99% or 100% sequence homology or identity with the sequence as set forth in at least one of SEQ ID Nos 16-37. Said probe, tool or reagent may be a primer. Said probe, tool or reagent may comprise an oligonucleotide having a nucleotide sequence as set forth in at least one of SEQ ID NOs: 16-37.

Suitable primers for identification of SNPs in the 16S rRNA sequence set forth in SEQ ID NO. 38 (especially for identification of the SNPs in Table 4) may be as shown in Table 7 below.

TABLE 7

| Forward primer sequence | Reverse primer sequence |
|---|---|
| 5′GTGTAGCGGTGAA | 5′TCGTTTACCGTGG |
| ATGCGTAGAG 3′ | ACTACCAGGG 3′ |
| (SEQ ID NO. 36) | (SEQ ID NO. 37) |

Any of the features described herein can be combined in any combination with any one or more of the other features described herein within the scope of the invention.

The above largely discusses the use of 16S rRNA genes. However, the above may also be applicable to 16S rRNA and to other 16S rRNA gene products. Accordingly, in some embodiments (and where appropriate), references to 16S rRNA gene above and below may be replaced with 16S rRNA gene product (or 16S rRNA).

The reference to any prior art in this specification is not, and should not be taken as an acknowledgement or any form of suggestion that the prior art forms part of the common general knowledge.

### BRIEF DESCRIPTION OF DRAWINGS

Various embodiments of the invention will be described with reference to the following drawings, in which:

FIG. 1 shows a standard curve created from control samples that correlates amplicon DNA (ng) to sample volume (uL).

FIG. 2 schematically demonstrates the spiking of control blood and serial dilution thereof to create a series of control samples of known bacterial concentration.

FIG. 3 provides the results of the comparative flow cytometry and plate count data for E. coli and S. aureus.

FIG. 4 shows high-resolution melt (HRM) curves of the spiked blood control sample for Bacteroides fragilis tested in Example 2;

FIG. 5 shows high-resolution melt (HRM) curves of the spiked blood control sample for Haemophilus influenzae tested in Example 2;

FIG. 6 shows high-resolution melt (HRM) curves of the spiked blood control sample for Pseudomonas aeruginosa tested in Example 2;

FIG. 7 shows high-resolution melt (HRM) curves of the spiked blood control sample for Streptococcus pneumoniae tested in Example 2;

FIG. 8 shows high-resolution melt (HRM) curves of the spiked blood control sample for Klebsiella pneumoniae tested in Example 2;

FIG. 9 shows high-resolution melt (HRM) curves of the spiked blood control sample for Escherichia coli tested in Example 2;

FIG. 10 shows high-resolution melt (HRM) curves of the spiked blood control sample for Enterobacter cloacae tested in Example 2;

FIG. 11 shows high-resolution melt (HRM) curves of the spiked blood control sample for Serratia marcescens tested in Example 2;

FIG. 12 shows high-resolution melt (HRM) curves of the spiked blood control sample for Proteus mirabilis tested in Example 2;

FIG. 13 is a CLUSTALW sequence alignment of the representative genes encoding 16S rRNA molecules from the following bacterial species: Acinetobacter calcoaceticus; Enterobacter aerogenes; Enterobacter cloacae; Enterococcus faecalis; Enterococcus faecium; Escherichia coli; Klebsiella pneumoniae; Proteus mirabilis; Pseudomonas aeruginosa; Serratia marcescens; Staphylococcus aureus; Staphylococcus epidermidis; Streptococcus agalactiae; Streptococcus pneumoniae; and Streptococcus pyogenes. Variable sequences, as determined by the CLUSTALW alignment were removed. The SNPs at positions corresponding to positions 273, 378, 408, 412, 440, 488, 647 and 653 of the 16S rRNA gene from E. coli as set forth in SEQ ID NO:1 are highlighted together with the corresponding nucleotide in the aligned sequences; and

FIG. 14 is an example of a typical standard curve of Cₜ versus log copy number (FIG. 14A) and illustrates Cₜ values obtained from amplification plots which indicate the change in normalized signal for the five standards (indicated with copy numbers) between cycles 20 and 40 of the PCR (FIG. 14B).

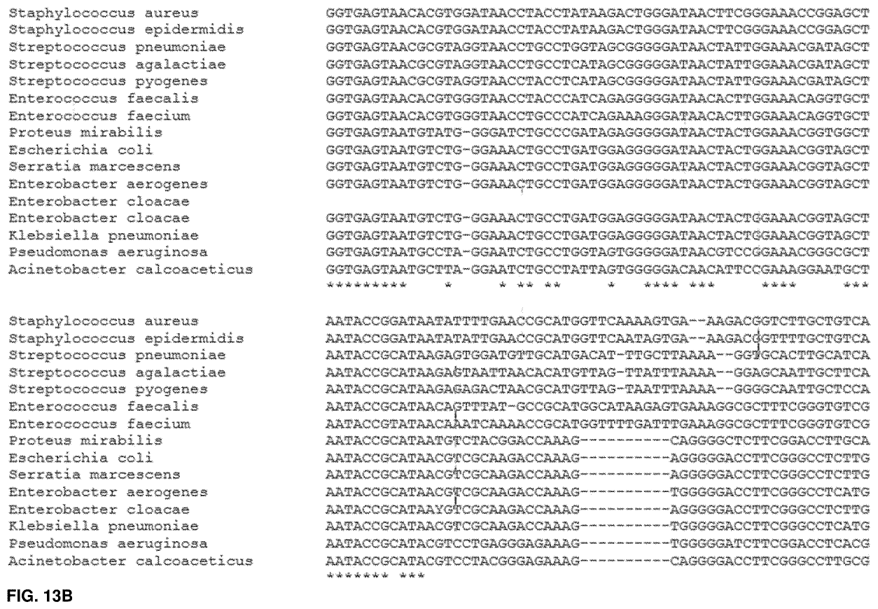

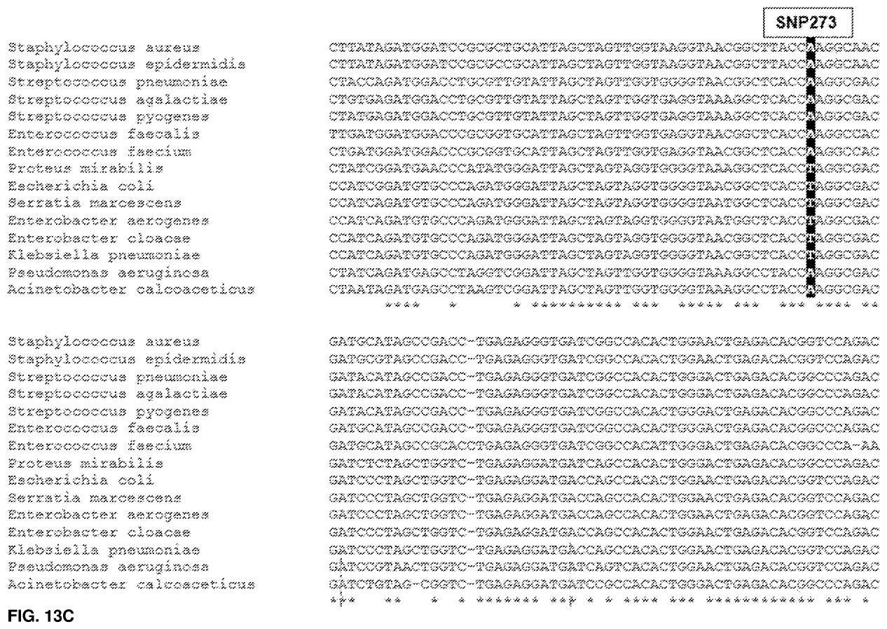

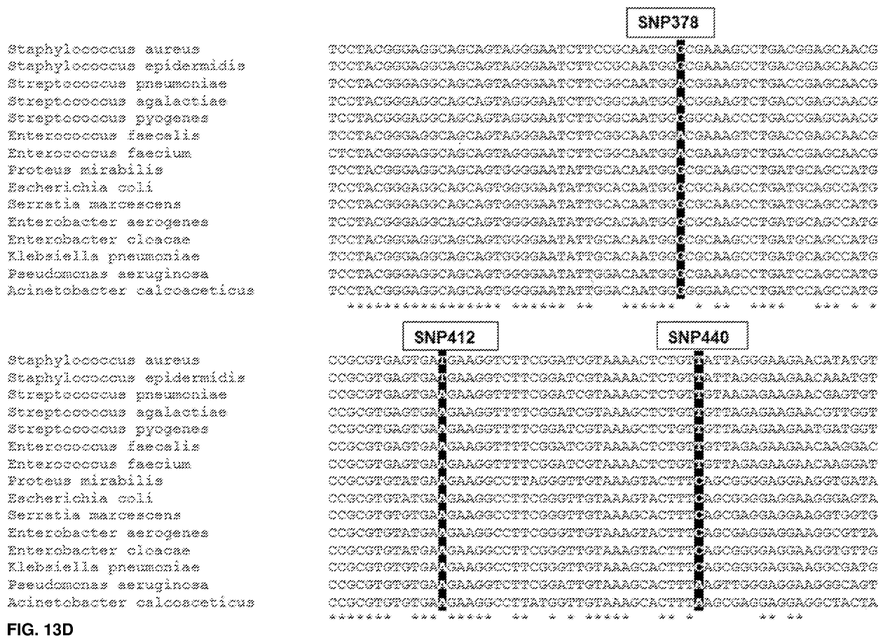

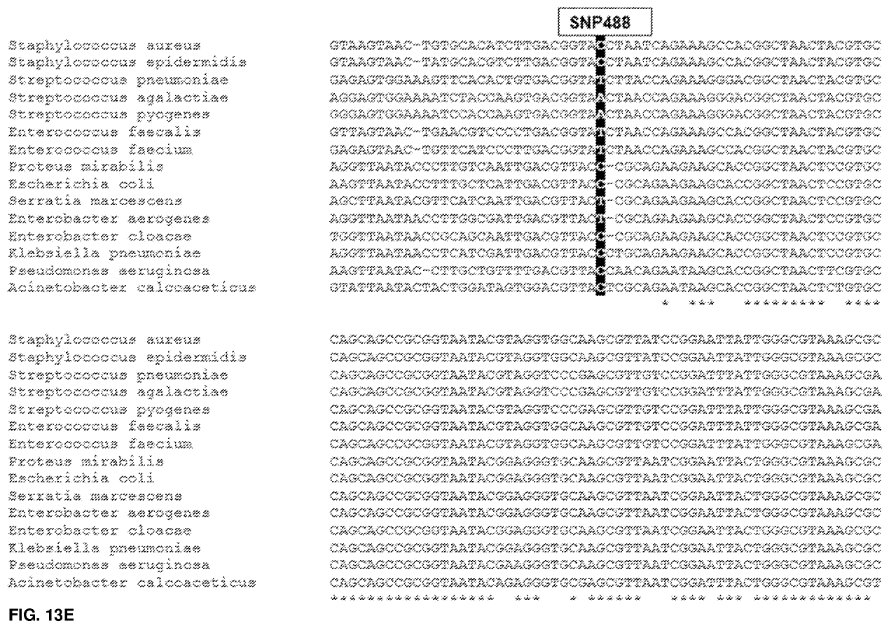

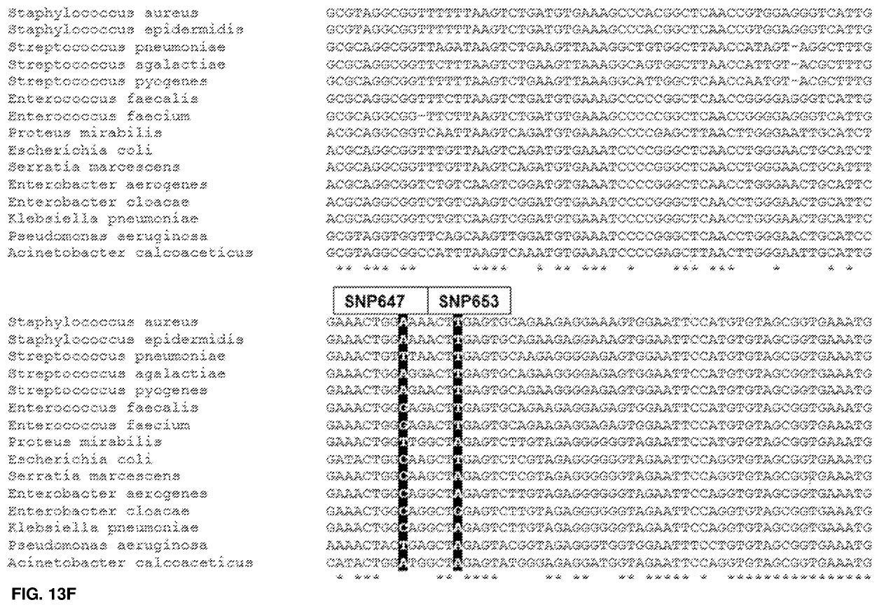

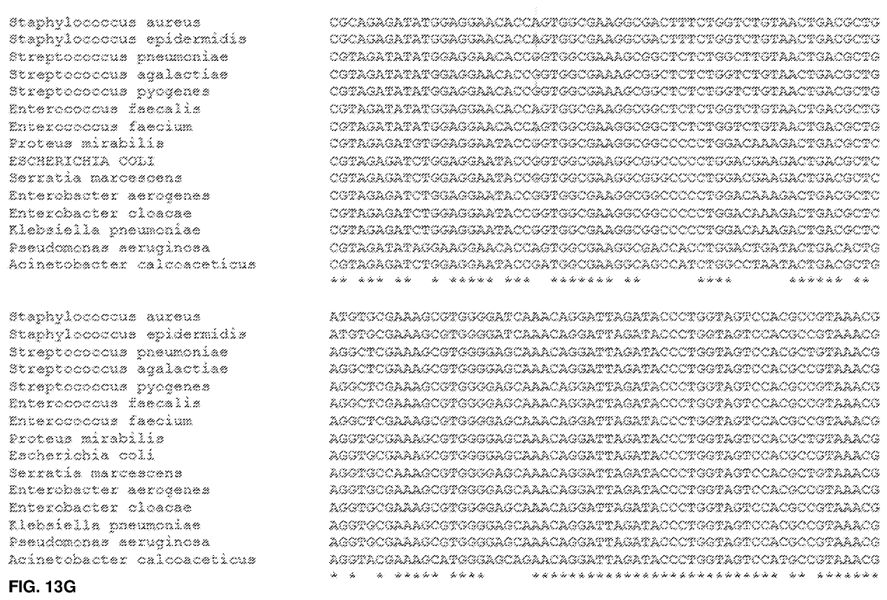

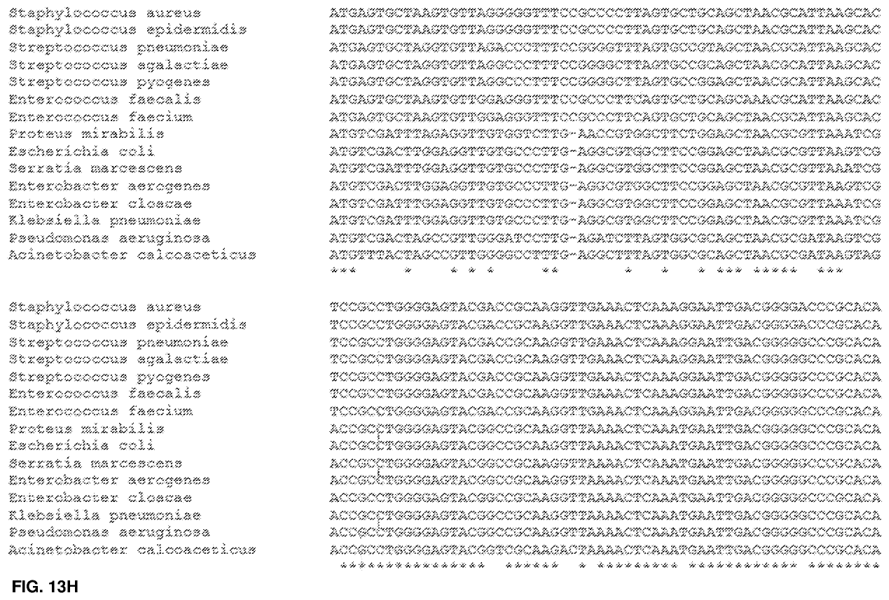

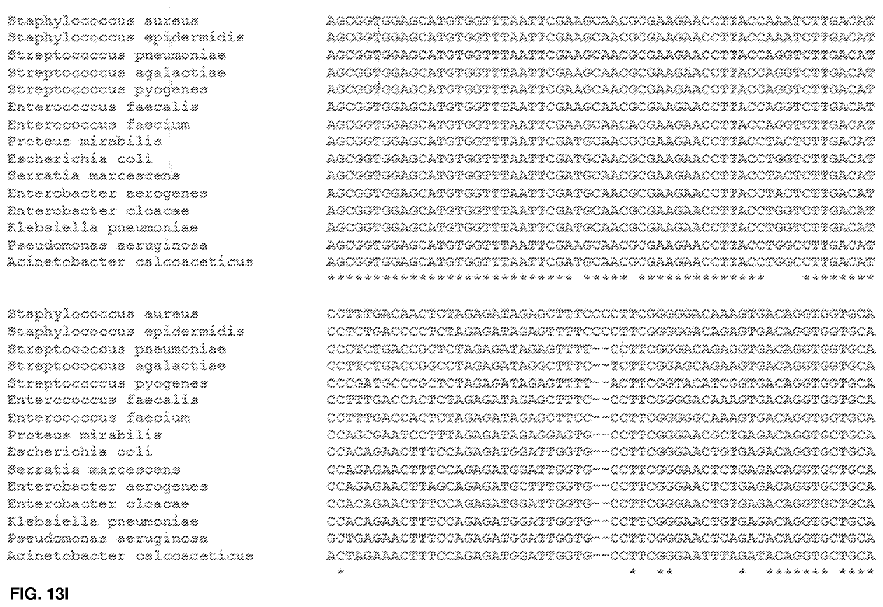

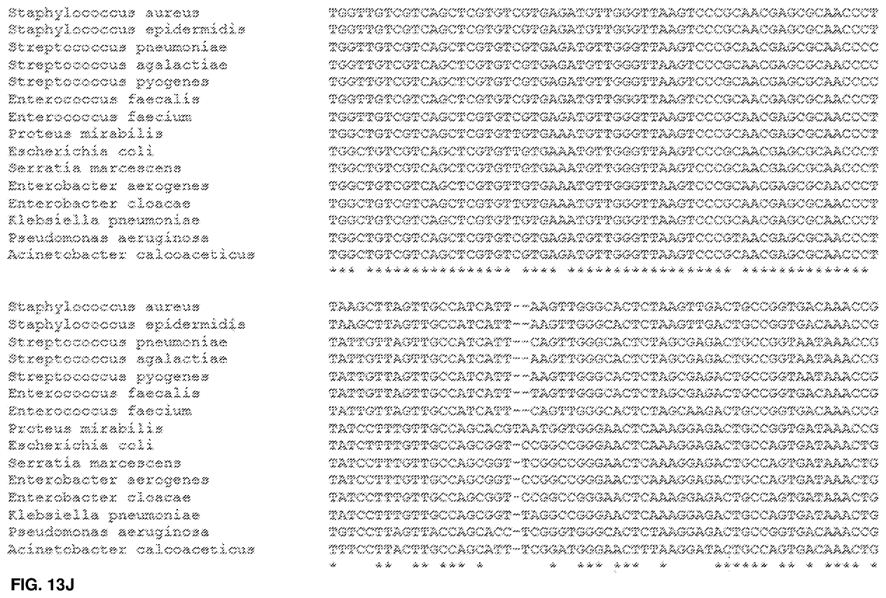

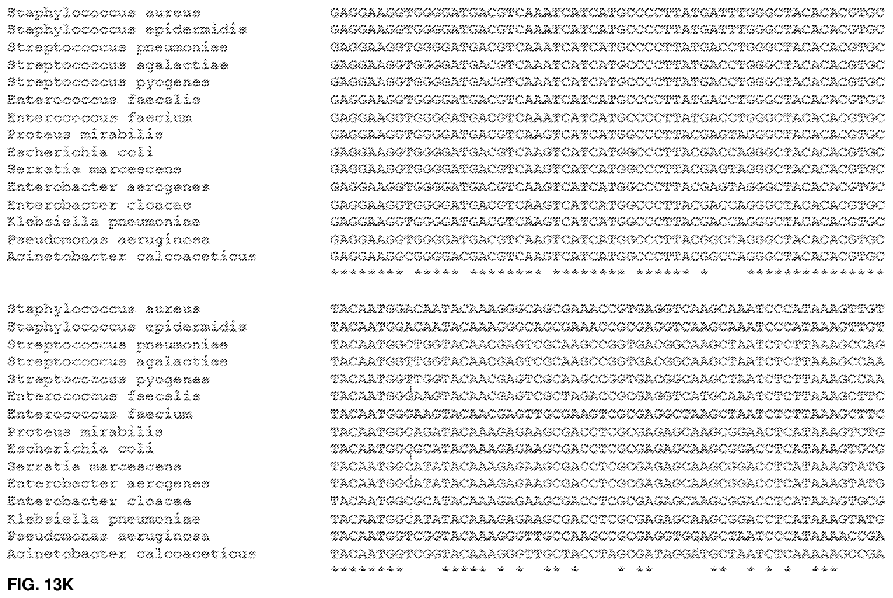

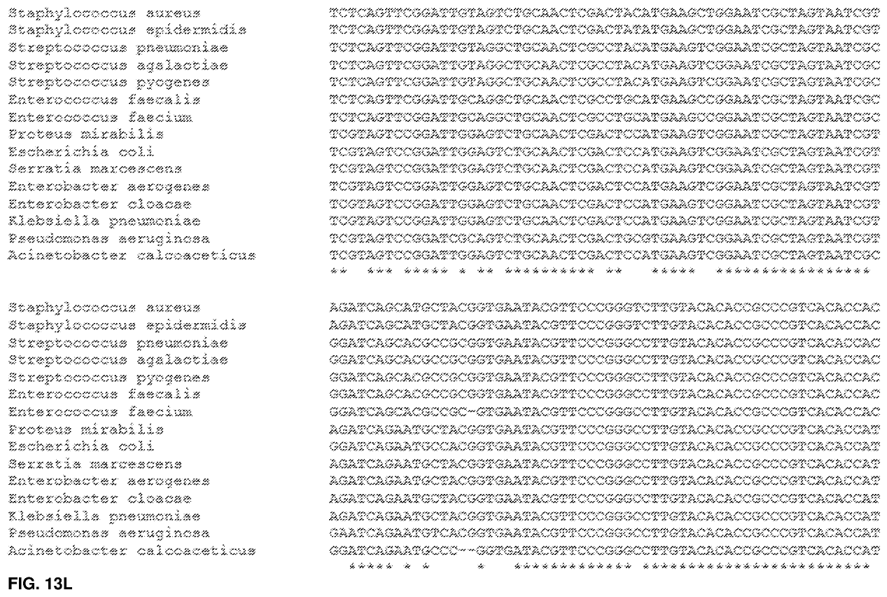

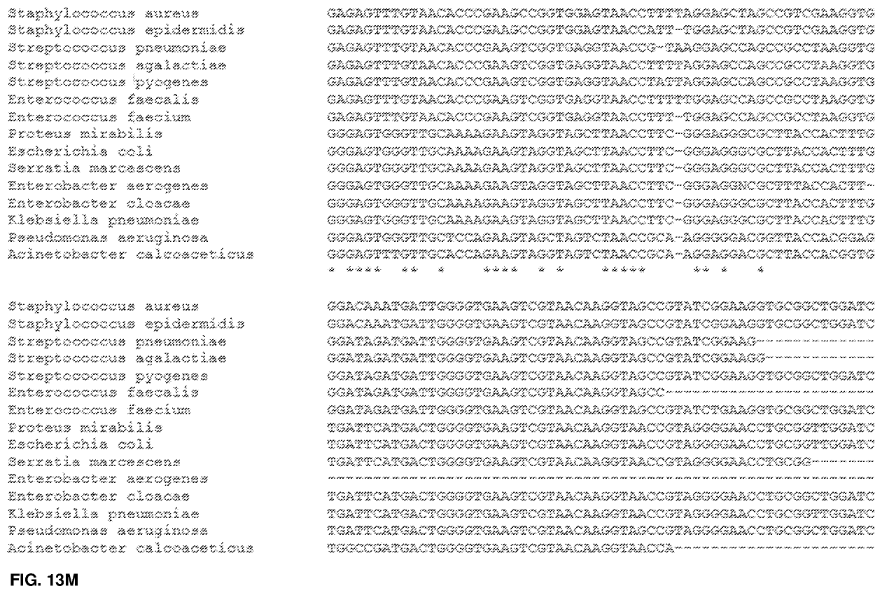

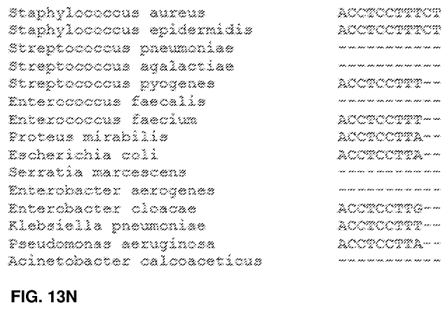

KEY TO SEQUENCE LISTING
SEQ ID NO: 1: Escherichia coli 16S rRNA gene in FIG. 13
(Genbank accession NR_102804.1);
SEQ ID NO: 2: Staphylococcus aureus 16S rRNA gene in FIG. 13
(Genbank accession NR_075000.1);
SEQ ID NO: 3: Staphylococcus epidermidis 16S rRNA gene in FIG. 13
(Genbank accession NR_074995.1);
SEQ ID NO: 4: Streptococcus pneumoniae 16S rRNA gene in FIG. 13
(Genbank accession NR_074564.1);
SEQ ID NO: 5: Streptococcus agalactiae 16S rRNA gene in FIG. 13
(Genbank accession NR_040821.1);
SEQ ID NO: 6: Streptococcus pyogenes 16S rRNA gene in FIG. 13
(Genbank accession NR_074091.1);
SEQ ID NO: 7: Enterococcus faecalis 16S rRNA gene in FIG. 13
(Genbank accession NR_074637.1);
SEQ ID NO: 8: Enterococcus faecium 16S rRNA gene in FIG. 13
(Genbank accession NR_042054.1);
SEQ ID NO: 9: Proteus mirabilis 16S rRNA gene in FIG. 13
(Genbank accession NR_074898.1);
SEQ ID NO: 10: Serratia marcescens 16S rRNA gene in FIG. 13
(Genbank accession NR_041980.1);
SEQ ID NO: 11: Enterobacter aerogenes 16S rRNA gene in FIG. 13
(Genbank accession NR_024643.1);
SEQ ID NO: 12: Enterobacter cloacae 16S rRNA gene in FIG. 13
(Genbank accession NR_028912.1);
SEQ ID NO: 13: Klebsiella pneumoniae 16S rRNA gene in FIG. 13
(Genbank accession NR_036794.1);
SEQ ID NO: 14: Pseudomonas aeruginosa 16S rRNA gene in FIG. 13
(Genbank accession NR_074828.1);
SEQ ID NO: 15: Acinetobacter calcoaceticus 16S rRNA gene in FIG. 13
(Genbank accession AB302132.1);
SEQ ID NO: 16: Forward Primer (CCTCTTGCCATCGGATGTG);
SEQ ID NO: 17: Reverse Primer (CCAGTGTGGCTGGTCATCCT);
SEQ ID NO: 18: Forward Primer (GGGAGGCAGCAGTAGGGAAT);
SEQ ID NO: 19: Forward Primer (CCTACGGGAGGCAGCAGTAG);
SEQ ID NO: 20: Reverse Primer (CGATCCGAAAACCTTCTTCACT);
SEQ ID NO: 21: Forward Primer (AAGACGGTCTTGCTGTCACTTATAGA);
SEQ ID NO: 22: Reverse Primer (CTATGCATCGTTGCCTTGGTAA);
SEQ ID NO: 23: Forward Primer (TGCCGCGTGAATGAAGAA);
SEQ ID NO: 24: Forward Primer (GCGTGAAGGATGAAGGCTCTA);
SEQ ID NO: 25: Forward Primer (TGATGAAGGTTTTCGGATCGT);
SEQ ID NO: 26: Reverse Primer (TGATGTACTATTAACACATCAACCTTCCT);
SEQ ID NO: 27: Reverse Primer (AACGCTCGGATCTTCCGTATTA);
SEQ ID NO: 28: Reverse Primer (CGCTCGCCACCTACGTATTAC);
SEQ ID NO: 29: Forward Primer (GTTGTAAGAGAAGAACGAGTGTGAGAGT);
SEQ ID NO: 30: Reverse Primer (CGTAGTTAGCCGTCCCTTTCTG);
SEQ ID NO: 31: Forward Primer (GCGGTTTGTTAAGTCAGATGTGAA);
SEQ ID NO: 32: Forward Primer (GGTCTGTCAAGTCGGATGTGAA);
SEQ ID NO: 33: Forward Primer (TCAACCTGGGAACTCATTCGA);
SEQ ID NO: 34: Reverse Primer (GGAATTCTACCCCCCTCTACGA);
SEQ ID NO: 35: Reverse Primer (GGAATTCTACCCCCCTCTACAAG);
SEQ ID NO: 36: Forward Primer (GTGTAGCGGTGAAATGCGTAGAG);
SEQ ID NO: 37: Reverse Primer (TCGTTTACCGTGGACTACCAGGG).
SEQ ID NO: 38: Bacillus anthracis strain 2000031664 16S ribosomal
RNA gene, partial sequence (GenBank accession AY138383.1);
TTATTGGAGA GTTTGATCCT GGCTCAGGAT GAACGCTGGC GGCGTGCCTA
ATACATGCAA GTCGAGCGAA TGGATTAAGA GCTTGCTCTT ATGAAGTTAG
CGGCGGACGG GTGAGTAACA CGTGGGTAAC CTGCCCATAA GACTGGGATA
ACTCCGGGAA ACCGGGGCTA ATACCGGATA ACATTTTGAA CCGCATGGTT
CGAAATTGAA AGGCGGCTTC GGCTGTCACT TATGGATGGA CCCGCGTCGC
ATTAGCTAGT TGGTGAGGTA ACGGCTCACC AAGGCAACGA TGCGTAGCCG
ACCTGAGAGG GTGATCGGCC ACACTGGGAC TGAGACACGG CCCAGACTCC
TACGGGAGGC AGCAGTAGGG AATCTTCCGC AATGGACGAA AGTCTGACGG
AGCAACGCCG CGTGAGTGAT GAAGGCTTTC GGGTCGTAAA ACTCTGTTGT
TAGGGAAGAA CAAGTGCTAG TTGAATAAGC TGGCACCTTG ACGGTACCTA
ACCAGAAAGC CACGGCTAAC TACGTGCCAG CAGCCGCGGT AATACGTAGG
TGGCAAGCGT TATCCGGAAT TATTGGGCGT AAAGCGCGCG CAGGTGGTTT
CTTAAGTCTG ATGTGAAAGC CCACGGCTCA ACCGTGGAGG GTCATTGGAA
ACTGGGAGAC TTGAGTGCAG AAGAGGAAAG TGGAATTCCA TGTGTAGCGG
TGAAATGCGT AGAGATATGG AGGAACACCA GTGGCGAAGG CGACTTTCTG
GTCTGTAACT GACACTGAGG CGCGAAAGCG TGGGGAGCAA ACAGGATTAG
ATACCCTGGT AGTCCACGCC GTAAACGATG AGTGCTAAGT GTTAGAGGGT
TTCCGCCCTT TAGTGCTGAA GTTAACGCAT TAAGCACTCC GCCTGGGGAG
TACGGCCGCA AGGCTGAAAC TCAAAGGAAT TGACGGGGGC CCGCACAAGC
GGTGGAGCAT GTGGTTTAAT TCGAAGCAAC GCGAAGAACC TTACCAGGTC
TTGACATCCT CTGACAACCC TAGAGATAGG GCTTCTCCTT CGGGAGCAGA
GTGACAGGTG GTGCATGGTT GTCGTCAGCT CGTGTCGTGA GATGTTGGGT
TAAGTCCCGC AACGAGCGCA ACCCTTGATC TTAGTTGCCA TCATTWAGTT
GGGCACTCTA AGGTGACTGC CGGTGACAAA CCGGAGGAAG GTGGGGATGA
CGTCAAATCA TCATGCCCCT TATGACCTGG GCTACACACG TGCTACAATG
GACGGTACAA AGAGCTGCAA GACCGCGAGG TGGAGCTAAT CTCATAAAAC
CGTTCTCAGT TCGGATTGTA GGCTGCAACT CGCCTACATG AAGCTGGAAT
CGCTAGTAAT CGCGGATCAG CATGCCGCGG TGAATACGTT CCCGGGCCTT
GTACACACCG CCCGTCACAC CACGAGAGTT TGTAACACCC GAAGTCGGTG
GGGTAACCTT TTTGGAGCCA GCCGCCTAAG GTGGGACAGA TGATTGGGGT
GAAGTCGTAA CAAGGTAGCC GTATCGGAAG GTGCGGCTGG ATCACCTCCT
TTCT
SEQ ID NO: 39: Burkholderia pseudomallei 16S rRNA gene
(GenBank accession AJ131790.1);
TCTAGATGCG TGCTCGAGCG GCCGCCCAGT GCTGCATGGA TATCTGCTGA
ATTCGGCTTG AGCAGTTTGA TCCTGGCTCA GATTGAACGC TGGCGGCATG
CCTTACACAT GCAAGTCGAA CGGCAGCACG GGCTTCGGCC TGGTGGCGAG
TGGCGAACGG GTGAGTTATA CATCGGAGCA TGTCCTGTAG TGGGGGATAG
CCCGGCGAAA GCCGAATTAA TACCGCATAC GATCTGAGGA TGAAAGCGGG
GGACCTTCGG GCCTCGCGCT ATAGGGTTGG CCGATGGCTG ATTAGCTAGT
TGGTGGGGTA AAGGCCTACC AAGGCGACGA TCAGTAGCTG GTCTGAGAGG
ACGACCAGCC ACACTGGGAC TGAGACACGG CCCAGACTCC TACGGGAGGC
AGCAGTGGGG AATTTTGGAC AATGGGCGCA AGCCTGATCC AGCAATGCCG
CGTGTGTGAA GAAGGCCTTC GGGTTGTAAA GCACTTTTGT CCGGAAAGAA
ATCATTCTGG CTAATACCCG GAGTGGATGA CGGTACCGGA AGAATAAGCA
CCGGCTAACT ACGTGCCAGC AGCCGCGGTA ATACGTAGGG TGCGAGCGTT
AATCGGGATT ACTGGGCGTA AAGCGTGCGC AGGCGGTTTG CTAAGACCGA
TGTGAAATCC CCGGGCTCAA CCTGGGAACT GCATTGGTGA CTGGCAGGCT
AGAGTATGGC AGAGGGGGGT AGAATTCCAC GTGTAGCAGT GAAATGCGTA
GAGATGTGGA GGAATACCGA TGGCGAAGGC AGCCCCCTGG GCCAATACTG
ACGCTCATGC ACGAAAGCGT GGGGAGAAAA CAGGATTAGA TACCCTGGTA
GTCCACGCCC TAAACGATGT CAACTAGTTG TTGGGGATTC ATTTCCTTAG
TAACGTAGCT AACGCGCGAA GTTGACCGCC TGGGGAGTAC GGTCGCAAGA
TTAAAACTCA AAGGAATTGA CGGGGACCCG CACAAGCGGT GGATGATGTG
GATTAATTCG ATGCAACGCG AAAAACCTTA CCTACCCTTG ACATGGTCGG
AAGCCCGATG AGAGTTGGGC GTGCTCGAAA GAGAACCGGC GCACAGGTGC
TGCATGGCTG TCGTCAGCTC GTGTCGTGAG ATGTTGGGTT AAGTCCCGCA
ACGAGCGCAA CCCTTGTCCT TAGTTGCTAC GCAAGAGCAC TCTAAGGAGA
CTGCCGGTGA CAAACCGGAG GAAGGTGGGG ATGACGTCAA GTCCTCATGG
CCCTTATGGG TAGGGCTTCA CACGTCATAC AATGGTCGGA ACAGAGGGTC
GCCAACCCGC GAGGGGGAGC CAATCCCAGA AAACCGATCG TAGTCCGGAT
TGCACTCTGC AACTCGAGTG CATGAAGCTG GAATCGCTAG TAATCGCGGA
TCAGCATGCC GCGGTGAATA CGTTCCCGGG TCTTGTACAC ACCGCCCGTC
ACACCATGGG AGTGGGTTTT ACCAGAAGTG GCTAGTCTAA CCGCAAGGAG
GACGGTCACC ACGGTAGGAT TCATGACTGG GGTGAAGTCG TAACAAGGTA
GCCGTAGAAG CCGAATTCCA GCACACTGGC GGCCGTTACT ACTGGATCCG
AGCTCGTACC
SEQ ID NO: 40: Clostridium botulinum type A rrn gene for 16S RNA
(GenBank accession X68185.1);
NNNNNNNGAG AGTTTGATCC TGGCTCAGGA CGAACGCTGG CGGCGTGCTT
AACACATGCA AGTCGAGCGA TGAAGCTTCC TTCGGGAAGT GGATTAGCGG
CGGACGGGTG AGTAACACGT GGGTAACCTG CCTCAAAGTG GGGGATAGCC
TTCCGAAAGG AAGATTAATA CCGCATAATA TAAGAGAATC GCATGATTTT
CTTATCAAAG ATTTATTGCT TTGAGATGGA CCCGCGGCGC ATTAGCTAGT
TGGTAAGGTA ACGGCTTACC AAGGCAACGA TGCGTAGCCG ACCTGAGAGG
GTGATCGGCC ACATTGGAAC TGAGACACGG TCCAGACTCC TACGGGAGGC
AGCAGTGGGG AATATTGCGC AATGGGGGAG ACCCTGACGC AGCAACGCCG
CGTGGGTGAT GAAGGTCTTC GGATTGTAAA GCCCTGTTTT CTAGGACGAT
AATGACGGTA CTAGAGGAGG AAGCCACGGC TAACTACGTG CCAGCAGCCG
CGGTAATACG TAGGTGGCGA GCGTTGTCCG GATTTACTGG GCGTAAAGGG
TGCGTAGGCG GATGTTTAAG TGGGATGTGA AATCCCCGGG CTTAACCTGG
GGGCTGCATT CCAAACTGGA TATCTAGAGT GCAGGAGAGG AAAGCGGAAT
TCCTAGTGTA GCGGTGAAAT GCGTAGAGAT TAGGAAGAAC ACCAGTGGCG
AAGGCGGCTT TCTGGACTGT AACTGACGCT GAGGCACGAA AGCGTGGGTA
GCAAACAGGA TTAGATACCC TGGTAGTCCA CGCCGTAAAC GATGGATACT
AGGTGTAGGG GGTATCAACT CCCCCTGTGC CGCAGTTAAC ACAATAAGTA
TCCCGCCTGG GGAGTACGGT CGCAAGATTA AAACTCAAAG GAATTGACGG
GGGCCCGCAC AAGCAGCGGA GCATGTGGTT TAATTCGAAG CAACGCGAAG
AACCTTACCT GGACTTGACA TCCCTTGCAT AGCCTAGAGA TAGGTGAAGC
CCTTCGGGGC AAGGAGACAG GTGGTGCATG GTTGTCGTCA GCTCGTGTCG
TGAGATGTTA GGTTAAGTCC TGCAACGAGC GCAACCCTTG TTATTAGTTG
CTACCATTAA GTTGAGCACT CTAATGAGAC TGCCTGGGTA ACCAGGAGGA
AGGTGGGGAT GACGTCAAAT CATCATGCCC CTTATGTCCA GGGCTACACA
CGTGCTACAA TGGTAGGTAC AATAAGACGC AAGACCGTGA GGTGGAGCAA
AACTTATAAA ACCTATCTCA GTTCGGATTG TAGGCTGCAA CTCGCCTACA
TGAAGCTGGA GTTGCTAGTA ATCGCGAATC AGAATGTCGC GGTGAATACG
TTCCCGGGCC TTGTACACAC CGCCCCGTCA CACCATGAGA GCTGGTAACA
CCCGAAGTCC GTGAGGTAAC CGTAAGGAGC CAGCGGCCGA AGGTGGGATT
AGTGATTGGG GTGAAGTCGT AACAAGGTAG CCGTAGGAGA ACCTGCGGCT
GGATCACCTC C
SEQ ID NO: 41: Clostridium botulinum type B rrn gene for 16S RNA
(GenBank accession X68186.1);
NNNNNNNGAG AGTTTGATCC TGGCTCAGGA CGAACGCTGG CGGCGTGCTT
AACACATGCA AGTCGAGCGA TGAAGCTTCC TTCGGGAAGT GGATTAGCGG
CGGACGGGTG AGTAACACGT GGGTAACCTG CCTCAAAGTG GGGGATAGCC
TTCCGAAAGG AAGATTAATA CCGCATAACA TAAGAGAATC GCATGATTTT
CTTATCAAAG ATTTATTGCT TTGAGATGGA CCCGCGGCGC ATTAGCTAGT
TGGTAAGGTA ACGGCTTACC AAGGCAACGA TGCGTAGCCG ACCTGAGAGG
GTGATCGGCC ACATTGGAAC TGAGACACGG TCCAGACTCC TACGGGAGGC
AGGAGTGGGG AATATTGCGC AATGGGGGAA ACCCTGACGC AGCAACGCCG
CGTGGGTGAT GAAGGTCTTC GGATTGTAAA GCCCTGTTTT CTAGGACGAT
AATGACGGTA CTAGAGGAGG AAGCCACGGC TAACTACGTG CCAGCAGCCG
CGGTAATACG TAGGTGGCGA GCGTTGTCCG GATTTACTGG GCGTAAAGGG
TGCGTAGGCG GATGTTTAAG TGGGATGTGA AATCCCCGGG CTTAACCTGG
GGGCTGCATT CCAAACTGGA TATCTAGAGT GCAGGAGAGG AAAGCGGAAT
TCCTAGTGTA GCGGTGAAAT GCGTAGAGAT TAGGAAGAAC ACCAGTGGCG
AAGGCGGCTT TCTGGACTGT AACTGACGCT GAGGCACGAA AGCGTGGGTA
GCAAACAGGA TTAGATACCC TGGTAGTCCA CGCCGTAAAC GATGGATACT
AGGTGTAGGG GGTATCAACT CCCCCTGTGC CGCAGTTAAC ACAATAAGTA
TCCCGCCTGG GGAGTACGGT CGCAAGATTA AAACTCAAAG GAATTGACGG
GGGCCCGCAC AAGCAGCGGA GCATGTGGTT TAATTCGAAG CAACGCGAAG
AACCTTACCT GGACTTGACA TCCCTTGCAT AGCCTAGAGA TAGGTGAAGC
CCTTCGGGGC AAGGAGACAG GTGGTGCATG GTTGTCGTCA GCTCGTGTCG
TGAGATGTTA GGTTAAGTCC TGCAACGAGC GCAACCCTTG TTATTAGTTG
CTACCATTAA GTTGAGCACT CTAATGAGAC TGCCTGGGTA ACCAGGAGGA
AGGTGGGGAT GACGTCAAAT CATCATGCCC CTTATGTCCA GGGCTACACA
CGTGCTACAA TGGTAGGTAC AATAAGACGC AAGACCGTGA GGTGGAGCAA
AACTTATAAA ACCTATCTCA GTTCGGATTG TAGGCTGCAA CTCGCCTACA
TGAAGCTGGA GTTGCTAGTA ATCGCGAATC AGAATGTCGC GGTGAATACG
TTCCCGGGCC TTGTACACAC CGCCCGTCAC ACCATGAGAG CTGGTAACAC
CCGAAGTCCG TGAGGTAACC GTAAGGAGCC AGCGGCCGAA GGTGGGATTA
GTGATTGGGG TGAAGTCGTA ACAAGGTAGC CGTAGGAGAA CCTGCGGCTG
GATCACCTCC
SEQ ID NO: 42: Clostridium botulinum type C rrn gene for 16S rRNA
(GenBank accession X68315.1);
NNNNNNNGAG AGTTTGATCC TGGCTCAGGA CGAACGTGGC GGCGTGCCTA
ACACATGCAA GTCGAGCGAT GAAGCTTCCT TCGGGGAGTG GATTAGCGGC
GGACGGGTGA GTAACACGTG GGTAACCTGC CTCAAAGAGG GGGATAGCCT
CCCGAAAGGG AGATTAATAC CGCATAACAT TATTTTATGG CATCATAGAA
TAATCAAAGG AGCAATCCGC TTTGATTATG GACCCGCGTC GCATTAGCTA
GTTGGTGAGG TAACGGCTCA CCAAGGCAAC GATGCGTAGC CGACCTGAGA
GGGTGATCGG CCACATTGGA ACTGAGACAC GGTCCAGACT CCTACGGGAG
GCAGCAGTGG GGAATATTGC GCAATGGGGG AAACCCTGAC GCAGCAACGC
CGCGTGAGTG ATGAAGGTTT TCGGATCGTA AAACTCTGTC TTTAGGGACG
ATAATGACGG TACCTAAGGA GGAAGCCACG GCTAACTACG TGCCAGCAGC
CGCGGTAATA CGTAGGTGGC AAGCGTTGTC CGGATTTACT GGGCGTAAAG
AGTATGTAGG TGGGTGCTTA AGTCAGATGT GAAATTCCCG GGCTTAACCT
GGGCGCTGCA TTTGAAACTG GGCATCTAGA GTGCAGGAGA GGAAAGTGGA
ATTCCTAGTG TAGCGGTGAA ATGCGTAGAG ATTAGGAAGA ACACCAGTGG
CGAAGGCGAC TTTCTGGACT GTAACTGACA CTGAGATACG AAAGCGTGGG
TAGCAAACAG GATTAGATCC CCCTGGTAGT CCACGCCGTA AACGATGAAT
ACTAGGTGTC GGGGGGTACC ACCCTCGGTG CCGCAGCAAA CGCATTAAGT
ATTCCGCCTG GGGAGTACGG TCGCAAGATT AAAACTCAAA GGAATTGACG
GGGACCCGCA CAAGCAGCGG AGCATGTGGT TTAATTCGAA GCAACGCGAA
GAACCTTACC TAGACTTGAC ATCTCCTGAA TTACTCTTAA TCGAGGAAGT
CCCTTCGGGG GACAGGAAGA CAGGTGGTGC ATGGTTGTCG TCAGCTCGTG
TCGTGAGATG TTGGGTTAAG TCCCGCAACG AGCGCAACCC TTATTGTTAG
TTGCTACTAT TAAGTTAAGC ACTCTAACGA GACTGCCGCG GTTAACGTAG
AGGAAGGTGG GGATGACGTC AAATCATCAT GCCCCTTATG TCTAGGGCTA
CACACGTGCT ACAATGGCTG GTACAACGAG CAGCAAACCC GCGAGGGGGA
GCAAAACTTG AAAGCCAGTC CCAGTTCGGA TTGTAGGCTG AAACTCGCCT
ACATGAAGTT GGAGTTGCTA GTAATCGCGG AATCAGCATG TCGCGGTGAA
TACGTCCCCG GGTCTTGTAC ACACCGCCCG TCACACCATG AGAGCCGGTA
ACACCCGAAG CCCGTGAGGT AACCGTAAGG AGCCAGCGGT CGAAGGTGGG
ATTGGTGATT GGGGTGAAGT CGTAACAAGG TAGCCGTAGG AGAACCTGCG
GTTGGATCAC CTCCTT
SEQ ID NO: 43: Clostridium botulinum type D rrn gene for 16S RNA
(GenBank accession X68187.1);
NNNNNNNGAG AGTTTGATCC TGGCTCAGGA CGAACGCTGG CGGCGTGCCT
AACACATGCA AGTCGAGCGA TGAAGCTTCC TTCGGGAAGT GGATTAGCGG
CGGACGGGTG AGTAACACGT GGGTAACCTG CCTCAAAGAG TGGGATAGCC
TCCCGAAAGG GAGATTAATA CCGCATAACA TTATTTTATG GCATCATACA
TAAAATAATC AAAGGAGCAA TCCGCTTTGA GATGGACCCG CGGCGCATTA
GCTAGTTGGT GAGGTAACGG CTCACCAAGG CAACGATGCG TAGCCGACCT
GAGAGGGTGA TCGGCCACAT TGGAACTGAG ACACGGTCCA GACTCCTACG
GGAGGCAGCA GTGGGGAATA TTNCGCAATG GGGGAAACCC TGACGCAGCA
ACGCCGCGTG AGTGATGAAG GTTTTCGGAT CGTAAAACTC TGTCTTTAGG
GACGATAATG ACGGTACCTA AGGAGGAAGC CACGGCTAAC TACGTGCCAG
CAGCCGCGGT AATACGTAGG TGGCAAGCGT TGTCCGGATT TACTGGGCGT
AAAGAGTATG TAGGTGGGTG CTTAAGTCAG ATGTGAAATT CCCGGGCTCA
ACCTGGGAGC TGCATTTGAA ACTGGGCATC TAGAGTGCAG GAGAGGAAAG
TGGAATTCCT AGTGTAGCGG TGAAATGCGT AGAGATTAGG AAGAACACCA
GTGGCGAAGG CGACTCTCTG GACTGTAACT GACACTGAGA TACGAAAGCG
TGGGTAGCAA ACAGGATTAG ATACCCTGGT AGTCCACGNN GTAAACGATG
AATACTAGGT GTCGGGGGGT ACCACCCTCG GTGCCGCAGC AAACGCATTA
AGTATTCCGC CTGGGAAGTA CGGTCGCAAG ATTAAAACTC AAAGGAATTG
ACGGGGCCCG CACAAGCAGC GGAGCATGTG GTTTAATTCG AAGCAACGCG
AAGAACCTTA CCTAGACTTG ACATCTCCTG AATTACTCTT AATCGAGGAA
GTCCCTTCGG GGACAGGAAG ACAGGTGGTG CATGGTTGTC GTCAGCTCGT
GTCGTGAGAT GTTGGGTTAA GTCCCGCAAC GAGCGCAACC CTTATTGTTA
GTTGCTACTA TTAAGTTAAG CACTCTAACG AGACTGCCGC GGTTAACGTG
GAGGAAGGTG GGGATGACGT CAAATCATCA TGCCCCTTAT GTCTAGGGCT
ACACACGTGC TACAATGGCT GGTACAACGA GCAGCAAACC CGCGAGGGGG
AGCAAAACTT GAAAGCCAGT CCCAGTTCGG ATTGTAGGCT GAAACTCGCC
TACATGAAGT TGGAGTTGCT AGTAATCGCG AATCAGCATG TCGCGGTGAA
TACGTTCCCG GGTCTTGTAC ACACCGCCCG TCACACCATG AGAGCCGGTA
ACACCCGAAG CCCGTGAGGT AACCGTAAGG AGCCAGCGGT CGAAGGTGGG
ATTGGTGATT GGGGTAAGTC GTAACAAGGT AGCCGTAGGA GAACCTGCGG
CTGGATCACC TCCTTT
SEQ ID NO: 44: Clostridium botulinum type G rrn gene for 16S rRNA
(GenBank accession X68317.1);
NNNNNNNAGA GTTTGATCCT GGCTCAGGAC GAACGCTGGC GGCGTGCCTA
ACACATGCAA TCGAGCGATG AAGCTTCCTT CGGGAAGTGG ATTAGCGGCG
GACGGGTGAG TAACACGTGG GTAACCTGCC TCATAGAGGG GAATAGCCTC
CCGAAAGGGA GATTAATACC GCATAAAGTA TGAAGGTCGC ATGACTTCAT
TATACCAAAG GAGTAATCCG CTATGAGATG GACCCGCGGC GCATTAGCTA
GTTGGTGAGG TAAGGGCTCA CCAAGGCAAC GATGCGTAGC CGACCTGAGA
GGGTGATCGG CCACATTGGA ACTGAGACAC GGTCCAGACT CCTACGGGAG
GCAGCAGTGG GGAATATTGC GCAATGGGGG AAACCCTGAC GCAGCAACGC
CGCGTGAATG AAGAAGGCCT TAGGGTTGTA AAGTTCTGTC ATATGGGAAG
ATAATGACGG TACCATATGA GGAAGCCACG GCTAACTACG TGCCAGCAGC
CGCGGTAATA CGTAGGTGGC AAGCGTTGTC CGGATTTACT GGGCGTAAAG
GATGCGTAGG CGGACATTTA AGTCAGATGT GAAATACCCG GGCTCAACTT
GGGTGCTGCA TTTGAAACTG GGTGTCTAGA GTGCAGGAGA GGAAAGCGGA
ATTCCTAGTG TAGCGGTGAA ATGCGTAGAG ATTAGGAAGA ACACCAGTGG
CGAAGGCGGC TTTCTGGACT GTAACTGACG CTGAGGCATG AAAGCGTGGG
GAGCAAACAG GATTAGATAC CCTGGTAGTC CACGCCGTAA ACGATGAATA
CTAGGTGTAG GAGGTATCGA CCCCTTCTGT GCCGCAGTTA ACACAATAAG
TATTCCGCCT GGGGAGTACG ATCGCAAGAT TAAAACTCAA AGGAATTGAC
GGGGGCCCGC ACAAGCAGCG GAGCATGTGG TTTAATTCGA AGCAACGCGA
AGAACCTTAC CTAGACTTGA CATCCCCTGA ATTACCTGTA ATGAGGGAAG
CCCTTCGGGG CAGGGAGACA GGTGGTGCAT GGTTGTCGTC AGCTCGTGTC
GTGAGATGTT GGGTTAAGTC CCGCAACGAG CGCAACCCTT ATCATTAGTT
GCTACCATTA AGTTGAGCAC TCTAGTGAGA CTGCCCGGGT TAACCGGGAG
GAAGGTGGGG ATGACGTCAA ATCATCATGC CCCTTATGTC TAGGGCTACA
CACGTGCTAC AATGGTTGGT ACAACAAGAT GCAAGACCGC GAGGTGGAGC
TAAACTTAAA AAACCAACCC AGTTCGGATT GTAGGCTGAA ACTCGCCTAC
ATGAAGCCGG AGTTGCTAGT AATCGCGAAT CAGCATGTCG CGGTGAATAC
GTTCCCGGGC CTTGTACACA CCGCCCGTCA CACCATGAGA GCTGGTAACA
CCCGAAGTCC GTGAGGTAAC CGTAAGGAGC CAGCGGCCGA AGGTGGGATT
AGTGATTGGG GTGAAGTCGT AACAAGGTAG CCGTAGGAGA ACCTGCGGTT
GGATCACCTC CTT
SEQ ID NO: 45: Francisella tularensis strain B-38 16S ribosomal RNA,
partial sequence (GenBank accession/NCBI Reference Sequence: NR_029362.1);
TTGAAGAGTT TGATCATGGC TCAGATTGAA CGCTGGTGGC ATGCTTAACA
CATGCAAGTC GAACGGTAAC AGGTCTTAGG ATGCTGACGA GTGGCGGACG
GGTGAGTAAC GCGTAGGAAT CTGCCCATTT GAGGGGGATA CCAGTTGGAA
ACGACTGTTA ATACCGCATA ATATCTGTGG ATTAAAGGTG GCTTTCGGCG
TGTCGCAGAT GGATGAGCCT GCGTTGGATT AGCTAGTTGG TGGGGTAAGG
GCCCACCAAG GCTACGATCC ATAGCTGATT TGAGAGGATG ATCAGCCACA
TTGGGACTGA GACACGGCCC AAACTCCTAC GGGAGGCAGC AGTGGGGAAT
ATTGGACAAT GGGGGCAACC CTGATCCAGC AATGCCATGT GTGTGAAGAA
GGCCCTAGGG TTGTAAAGCA CTTTAGTTGG GGAGGAAAGC CTCAAGGTTA
ATAGCCTTGG GGGAGGACGT TACCCAAAGA ATAAGCACCG GCTAACTCCG
TGCCAGCAGC CGCGGTAATA CGGGGGGTGC AAGCGTTAAT CGGAATTACT
GGGCGTAAAG GGTCTGTAGG TGGTTTGTTA AGTCAGATGT GAAAGCCCAG
GGCTCAACCT TGGAACTGCA TTTGATACTG GCAAACTAGA GTACGGTAGA
GGAATGGGGA ATTTCTGGTG TAGCGGTGAA ATGCGTAGAG ATCAGAAGGA
ACACCAATGG CGAAGGCAAC ATTCTGGACC GATACTGACA CTGAGGGACG
AAAGCGTGGG GATCAAACAG GATTAGATAC CCTGGTAGTC CACGCTGTAA
ACGATGAGTA CTAGCTGTTG GAGTCGGTGT AAAGGCTCTA GTGGCGCACG
TAACGCGATA AGTACTCCGC CTGGGGACTA CGGCCGCAAG GCTAAAACTC
AAAGGAATTG ACGGGGACCC GCACAAGCGG TGGAGCATGT GGTTTAATTC
GATGCAACGC GAAGAACCTT ACCTGGTCTT GACATCCTGC GAACTTTCTA
GAGATAGATT GGTGCTTCGG AACGCAGTGA CAGTGCTGCA CGGCTGTCGT
CAGCTCGTGT TGTGAAATGT TGGGTTAAGT CCCGCAACGA GCGCAACCCC
TATTGATAGT TACCATCATT AAGTTGGGTA CTCTATTAAG ACTGCCGCTG
ACAAGGCGGA GGAAGGTGGG GACGACGTCA AGTCATCATG GCCCTTACGA
CCAGGGCTAC ACACGTGCTA CAATGGGTAT TACAGAGGGC TGCGAAGGTG
CGAGCTGGAG CGAAACTCAA AAAGGTACTC TTAGTCCGGA TTGCAGTCTG
CAACTCGACT GCATGAAGTC GGAATCGCTA GTAATCGCAG GTCAGAATAC
TGCGGTGAAT ACGTTCCCGG GTCTTGTACA CACCGCCCGT CACACCATGG
GAGTGGGTTG CTCCAGAAGT AGATAGCTTA ACGAATGGGC GTTTACCACG
GAGTGATTCA TGACTGGGGT GAAGTCGTAA CAATGGTAGC CGTAGGGAAC
CTGCGGCTGG ATCACCTCCT T
SEQ ID NO: 46: Vibrio cholerae strain DL2 16S ribosomal RNA gene,
partial sequence (GenBank accession MG062858.1);
TGGCTCAGAT TGAACGCTGG CGGCAGGCCT AACACATGCA AGTCGAGCGG
CAGCACAGAG GAACTTGTTC CTTGGGTGGC GAGCGGCGGA CGGGTGAGTA
ATGCCTGGGA AATTGCCCGG TAGAGGGGGA TAACCATTGG AAACGATGGC
TAATACCGCA TAACCTCGTA AGAGCAAAGC AGGGGACCTT CGGGCCTTGC
GCTACCGGAT ATGCCCAGGT GGGATTAGCT AGTTGGTGAG GTAAGGGCTC
ACCAAGGCGA CGATCCCTAG CTGGTCTGAG AGGATGATCA GCCACACTGG
AACTGAGACA CGGTCCAGAC TCCTACGGGA GGCAGCAGTG GGGAATATTG
CACAATGGGC GCAAGCCTGA TGCAGCCATG CCGCGTGTAT GAAGAAGGCC
TTCGGGTTGT AAAGTACTTT CAGTAGGGAG GAAGGTGGTT AAGCTAATAC
CTTAATCATT TGACGTTACC TACAGAAGAA GCACCGGCTA ACTCCGTGCC
AGCAGCCGCG GTAATACGGA GGGTGCAAGC GTTAATCGGA ATTACTGGGC
GTAAAGCGCA TGCAGGTGGT TTGTTAAGTC AGATGTGAAA GCCCTGGGCT
CAACCTAGGA ATCGCATTTG AAACTGACAA GCTAGAGTAC TGTAGAGGGG
GGTAGAATTT CAGGTGTAGC GGTGAAATGC GTAGAGATCT GAAGGAATAC
CGGTGGCGAA GGCGGCCCCC TGGACAGATA CTGACACTCA GATGCGAAAG
CGTGGGGAGC AAACAGGATT AGATACCCTG GTAGTCCACG CCGTAAACGA
TGTCTACTTG GAGGTTGTGA CCTAGAGTCG TGGCTTTCGG AGCTAACGCG
TTAAGTAGAC CGCCTGGGGA GTACGGTCGC AAGATTAAAA CTCAAATGAA
TTGACGGGGG CCCGCACAAG CGGTGGAGCA TGTGGTTTAA TTCGATGCAA
CGCGAAGAAC CTTACCTACT CTTGACATCC TCAGAAGAGA CTGGAGACAG
TCTTGTGCCT TCGGGAACTG AGAGACAGGT GCTGCATGGC TGTCGTCAGC
TCGTGTTGTG AAATGTTGGG TTAAGTCCCG CAACGAGCGC AACCCTTATC
CTTGTTTGCC AGCACGTAAT GGTGGGAACT CCAGGGAGAC TGCCGGTGAT
AAACCGGAGG AAGGTGGGGA CGACGTCAAG TCATCATGGC CCTTACGAGT
AGGGCTACAC ACGTGCTACA ATGGCGTATA CAGAGGGCAG CGATACCGCG
AGGTGGAGCG AATCTCACAA AGTACGTCGT AGTCCGGATT GGAGTCTGCA
ACTCGACTCC ATGAAGTCGG AATCGCTAGT AATCGCAAAT CAGAATGTTG
CGGTGAATAC GTTCCCGGGC CTTGTACACA CCGCCCGTCA CACCATGGGA
GTGGGCTGCA AAAGAAGCAG GTAGTTTAAC CTTCGGGAGG ACGCTTGCCA
CTTTGGGTAC TTGG
SEQ ID NO: 47: Yersinia pestis 16S rRNA gene, isolate:
SS-Yp-116 (GenBank accession AJ232238.1);
CTGGCGGCAG GCCTAACACA TGCAAGTCGA GCGGCACCGG GAAGTAGTTT
ACTACTTTGC CGGCGAGCGG CGGACGGGTG AGTAATGTCT GGGGATCTGC
CTGATGGAGG GGGATAACTA CTGGAAACGG TAGCTAATAC CGCATGACCT
CGCAAGAGCA AAGTGGGGGA CCTTAGGGCC TCACGCCATC GGATGAACCC
AGATGGGATT AGCTAGTAGG TGGGGTAATG GCTCACCTAG GCGACGATCC
CTAGCTGGTC TGAGAGGATG ACCAGCCACA CTGGAACTGA GACACGGTCC
AGACTCCTAC GGGAGGCAGC AGTGGGGAAT ATTGCACAAT GGGCGCAAGC
CTGATGCAGC CATGCCGCGT GTGTGAAGAA GGCCTTCGGG TTGTAAAGCA
CTTTCAGCGA GGAGGAAGGG GTTGAGTTTA ATACGCTCAA TCATTGACGT
TACTCGCAGA AGAAGCACCG GCTAACTCCG TGCCAGCAGC CGCGGTAATA
CGGAGGGTGC AAGCGTTAAT CGGAATTACT GGGCGTAAAG CGCACGCAGG
CGGTTTGTTA AGTCAGATGT GAAATCCCCG CGCTTAACGT GGGAACTGCA
TTTGAAACTG GCAAGCTAGA GTCTTGTAGA GGGGGGTAGA ATTCCAGGTG
TAGCGGTGAA ATGCGTAGAG ATCTGGAGGA ATACCGGTGG CGAAGGCGGC
CCCCTGGACA AAGACTGACG CTCAGGTGCG AAAGCGTGGG GAGCAAACAG
GATTAGATAC CCTGGTAGTC CACGCTGTAA ACGATGTCGA CTTGGAGGTT
GTGCCCTTGA GGCGTGGCTT CCGGAGCTAA CGCGTTAAGT CGACCGCCTG
GGGAGTACGG CCGCAAGGTT AAAACTCAAA TGAATTGACG GGGGCCCGCA
CAAGCGGTGG AGCATGTGGT TTAATTCGAT GCAACGCGAA GAACCTTACC
TACTCTTGAC ATCCACAGAA TTTGGCAGAG ATGCTAAAGT GCCTTCGGGA
ACTGTGAGAC AGGTGCTGCA TGGCTGTCGT CAGCTCGTGT TGTGAAATGT
TGGGTTAAGT CCCGCAACGA GCGCAACCCT TATCCTTTGT TGCCAGCACG
TAATGGTGGG AACTCAAGGG AGACTGCCGG TGACAAACCG GAGGAAGGTG
GGGATGACGT CAAGTCATCA TGGCCCTTAC GAGTAGGGCT ACACACGTGC
TACAATGGCA GATACAAAGT GAAGCGAACT CGCGAGAGCC AGCGGACCAC
ATAAAGTCTG TCGTAGTCCG GATTGGAGTC TGCAACTCGA CTCCATGAAG
TCGGAATCGC TAGTAATCGT AGATCAGAAT GCTACGGTGA ATACGTTCCC
GGGCCTTGTA CACACCGCCC GTCACACCAT GGGAGTGGGT TGCAAAAGAA
GTAGGTAGCT TAACCTTCGG GAGGGCGCTT ACCACTTTGT GATTCATGAC
TGGGGTGAAG TCGTAACAA
SEQ ID NO: 48: Forward Primer (TCCTACGGGAGGCAGCAGTAGGG);
SEQ ID NO: 49: Reverse Primer (CCGCTACACATGGAATTCCAC);
SEQ ID NO: 50: Forward Primer (GACTCCTACGGGAGGCAGCAGTGGG);
and
SEQ ID NO: 51: Reverse Primer (GGTATTAACTTACTGCCCTTCCTCCC).

### DETAILED DESCRIPTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in practice or testing of the present invention, preferred methods and materials are described. For the purposes of the present invention, the following terms are defined below.

The articles “a” and “an” are used herein to refer to one or to more than one (i.e., to at least one) of the grammatical objection of the article. By way of example, “an element” means one element or more than one element.

“Amplification product” or “amplicon” refers to a nucleic acid product generated by nucleic acid amplification techniques.

The term “biological sample” as used herein refers to a sample that may be extracted, untreated, treated, diluted or concentrated from a patient or subject. Suitably, the biological sample is selected from any part of a patient or subject's body, including, but not limited to, hair, skin, nails, tissues or bodily fluids such as sputum, saliva, cerebrospinal fluid, urine and blood.

In the present specification and claims (if any), the word “comprising” and its derivatives including “comprises” and “comprise” include each of the stated integers but does not exclude the inclusion of one or more further integers.

The term “copy number” herein refers to the number of copies of a nucleic acid sequence, such as a 16S rRNA gene or portion thereof, present in a test sample.

As used herein, “corresponding” nucleic acid positions or nucleotides refer to positions or nucleotides that occur at aligned loci of two or more nucleic acid molecules. Related or variant polynucleotides can be aligned by any method known to those of skill in the art. Such methods typically maximise matches, and include methods such as using manual alignments and by using the numerous alignment programs available (e.g., BLASTN) and others known to those of skill in the art. By aligning the sequences of polynucleotides, one skilled in the art can identify corresponding nucleotides or positions using identical nucleotides as guides. For example, by aligning sequences of the gene encoding the E. coli 16S rRNA (set forth in SEQ ID NO:1) with a gene encoding a 16S rRNA from another species, one of skill in the art can identify corresponding positions and nucleotides using conserved nucleotides as guides.

By “gene” is meant a unit of inheritance that occupies a specific locus on a genome and consists of transcriptional and/or translational regulatory sequences and/or a coding region and/or non-translated sequences (i.e., introns, 5′ and 3′ untranslated sequences).

By “gene product” is meant a product of the gene. For example, a gene product of the 16S rRNA gene includes 16S rRNA. Gene products also include, for example, cDNA sequences derived from the rRNA sequences. Gene products may also include products of the rRNA in which a SNP in the rRNA gene would result in a corresponding change in the product.

As used herein, the term “gene copy number” refers to the copy number of a nucleic acid molecule in a cell. The gene copy number includes the gene copy number in the genomic (chromosomal) DNA of a cell. In bacteria, in a normal state, the number of copies of a nucleic acid, such as the 16S rRNA gene, can vary between species. Therefore, the copy number for the 16S rRNA gene can be 1, 2, 3, 4, 5, 6, 7, 8, 9, 10, 11, 12, 13, 14, 15 etc depending upon the specific bacterium in question.

“Homology” refers to the percentage number of nucleic acids or amino acids that are identical or constitute conservative substitutions. Homology can be determined using sequence comparison programs such as GAP (Deveraux et al. 1984), which is incorporated herein by reference. In this way, sequences of a similar or substantially different length to those cited herein could be compared by insertion of gaps into the alignment, such gaps being determined, for example, by the comparison algorithm used by GAP.

“Hybridization” is used herein to denote the pairing of complementary nucleotide sequences to produce a DNA-DNA hybrid or a DNA-RNA hybrid. In DNA, A pairs with T and C pairs with G. In RNA, U pairs with A and C pairs with G. In this regard, the terms “match” and “mismatch” as used herein refer to the hybridization potential of paired nucleotides in complementary nucleic acid strands. Matched nucleotides hybridize efficiently, such as the classical A-T and G-C base pair mentioned above. Mismatches are other combinations of nucleotides that do not hybridize efficiently. The nucleotide symbols are set forth in Table 8:

TABLE 8
Nucleotide Symbols

| Symbol | Description |
|---|---|
| A | Adenosine |
| C | Cytidine |
| G | Guanosine |
| T | Thymidine |
| U | Uridine |
| M | Amino (adenosine, cytosine) |
| K | Keto (guanosine, thymidine) |
| R | Purine (adenosine, guanosine) |
| Y | Pyrimidine (cytosine, thymidine) |
| N | Any nucleotide |

By “isolated” is meant material that is substantially or essentially free from components that normally accompany it in its native state.

The term “oligonucleotide” as used herein refers to a polymer composed of a multiplicity of nucleotide residues (deoxynucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotide residues and linkages between them are naturally occurring, it will be understood that the term also includes within its scope various analogues including, but not restricted to, peptide nucleic acids (PNAs), phosphoramidates, phosphorothioates, methyl phosphonates, 2-O-methyl ribonucleic acids, and the like. The exact size of the molecule can vary depending on the particular application. An oligonucleotide is typically rather short in length generally from about 10 to 30 nucleotide residues, but the term can refer to molecules of any length, although the term “polynucleotide” or “nucleic acid” is typically used for large oligonucleotides.

The terms “patient” and “subject” are used interchangeably and refer to patients and subjects of human or other mammal and includes any individual being examined or treated using the methods of the invention. However, it will be understood that “patient” does not imply that symptoms are present. Suitable mammals that fall within the scope of the invention include, but are not restricted to, primates, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., koalas, bears, wild cats, wild dogs, wolves, dingoes, foxes and the like).

The term “polymorphism” as used herein refers to a difference in the nucleotide or amino acid sequence of a given region as compared to a nucleotide or amino acid sequence in a homologous-region of another individual, in particular, a difference in the nucleotide or amino acid sequence of a given region which differs between individuals of the same species. A polymorphism is generally defined in relation to a reference sequence. Polymorphisms include single nucleotide differences, differences in more than one nucleotide, and single or multiple nucleotide insertions, inversions and deletions; as well as single amino acid differences, differences in sequence of more than one amino acid, and single or multiple amino acid insertions, inversions and deletions. A “polymorphic site” is the locus at which variation occurs. It shall be understood that where a polymorphism is present in a nucleic acid sequence, and reference is made to the presence of a particular base or bases at a polymorphic site, the present invention encompasses the complementary base or bases on the complementary strand at that site.

The term “polynucleotide” or “nucleic acid” as used herein designates mRNA, RNA, rRNA, cRNA, cDNA, or DNA. The term typically refers to oligonucleotides greater than 30 nucleotides residues in length.

By “primer” it is meant an oligonucleotide which, when paired with a strand of DNA, is capable of initiating the synthesis of a primer extension product in the presence of a suitable polymerizing agent. The primer is preferably a single-stranded for maximum efficiency in amplification but can alternatively be double-stranded. A primer must be sufficiently long to prime the synthesis of extension products in the presence of the polymerization agent. The length of the primer depends on many factors, including application, temperature to be employed, template reaction conditions, other reagents, and source of primers. For example, depending on the complexity of the target sequence, the oligonucleotide primer typically contains 15 to 35 or more nucleotide residues, although it can contain fewer nucleotide residues. Primers can be large polynucleotides, such as from about 200 nucleotides to several kilobases or more. Primers can be selected to be “substantially complementary” to the sequence on the template to which it is designed to hybridize and serve as a site for the initiation of synthesis. By “substantially complementary” it is meant that the primer is sufficiently complementary to hybridize with a target polynucleotide. In some embodiments, the primer contains no mismatches with the template to which it is designed to hybridize but this is not essential. For example, non-complementary nucleotide residues can be attached to the 5′ end of the primer, with the remainder of the primer sequence being complementary to the template. Alternatively, non-complementary nucleotide residues or a stretch of non-complementary nucleotide residues can be interspersed into a primer, provided that the primer sequence has sufficient complementarity with the sequence of the template to hybridize therewith and thereby form a template for synthesis of the extension product of the primer.

“Probe” refers to a molecule that binds to a specific sequence or sub-sequence or other moiety of another molecule. Unless otherwise indicated, the term “probe” typically refers to a polynucleotide probe that binds to another polynucleotide, often called the “target polynucleotide”, through complementary base pairing. Probes can bind target polynucleotides lacking complete sequence complementarity with the probe, depending on the stringency of the hybridization conditions. Probes can be labelled directly or indirectly.

The terms “prognosis” and “prognostic” are used herein to include making a prognosis, which can provide for predicting a clinical outcome (with or without medical treatment), selecting an appropriate course of treatment (or whether treatment would be effective) and/or monitoring a current treatment and potentially changing the treatment. This may be at least partly based on quantifying the bacterium by the methods of the invention, which may be in combination with also identifying the bacterium. A prognosis may also include a prediction, forecast or anticipation of any lasting or permanent physical or psychological effects of the infection suffered by the subject after the bacterial infection has been successfully treated or otherwise resolved. Furthermore, prognosis may include one or more of determining sepsis potential or occurrence, therapeutic responsiveness, implementing appropriate treatment regimes, determining the probability, likelihood or potential for infection recurrence after therapy and prediction of development of resistance to established therapies (e.g., antibiotics). It would be appreciated that a positive prognosis typically refers to a beneficial clinical outcome or outlook, such as long-term survival without recurrence of the subject's bacterial infection, whereas a negative prognosis typically refers to a negative clinical outcome or outlook, such as recurrence or progression of the bacterial infection.

The term “quantify”, as used herein, refers to the measurement, calculation or estimation of the quantity or concentration, preferably in a quantitative, semi-quantitative or relative manner of a product, such as an amplification product, a target nucleic acid, a copy number of a 16S rRNA gene and/or a bacterium.

“Resistance” as used herein refers to a diminished or failed response of an organism, disease, tissue or cell, such as bacteria, to the intended effectiveness of a treatment, such as a chemical or drug (e.g., antibiotics). Resistance to a treatment can be already present at diagnosis or the start of treatment (i.e., intrinsic resistance) or it can develop with or after treatment (i.e., acquired resistance).

The term “sepsis” is used herein in accordance with its normal meaning in clinical medicine, and includes, for example systemic and/or blood-borne infections, such as bacterial infections.

The term “sepsis-associated bacteria” refers to bacteria that have been identified as being able to cause sepsis in a subject, or have been identified in the blood of a subject with sepsis. “Mammalian (e.g., human) sepsis-associated bacteria” therefore refers to bacteria that have been identified as being able to cause sepsis in a mammalian (e.g., human) subject, or have been identified in the blood of a mammalian (e.g., human) subject with sepsis. Examples of mammalian (e.g., human) sepsis-associated bacteria include Acinetobacter baumannii, Actinobacillus hominis, Actinomyces massiliensis, Aeromonas hydrophila, Bacillus anthracis, Bacteroides fragilis, Brucella abortus, Burkholderia cepacia, Campylobacter coli, Campylobacter fetus, Campylobacter jejuni, Campylobacter lari, Cardiobacterium valvarum, Chlamydia trachomatis, Chlamydophila abortus, Chlamydophila pneumoniae, Citrobacter freundii, Clostridium difficile, Clostridium perfringens, Corynebacterium diphtheriae, Corynebacterium jeikeium, Corynebacterium urealyticum, Dermatophilus congolensis, Edwardsiella tarda, Enterobacter aerogenes, Enterobacter cloacae, Enterococcus faecalis, Enterococcus faecium, Erysipelothrix rhusiopathiae, Escherichia coli, Eubacterium desmolans, Flavobacterium ceti, Haemophilus ducreyi, Haemophilus influenzae, Haemophilus parahaemolyticus, Haemophilus parainfluenzae, Helicobacter cinaedi, Helicobacter pylori, Klebsiella oxytoca, Klebsiella pneumonia, Lactobacillus intestinalis, Legionella pneumophila, Leptospira interrogans, Listeria monocytogenes, Micrococcus luteus, Mobiluncus curtisii, Moraxella catarrhalis, Morganella morganii, Mycobacterium tuberculosis, Neisseria gonorrhoeae, Neisseria meningitidis, Nocardia asteroids, Nocardia brasiliensis, Pasteurella multocida, Peptostreptococcus stomatis, Porphyromonas gingivalis, Prevotella buccae, Prevotella intermedia, Prevotella melaninogenica, Proteus mirabilis, Providencia alcalifaciens, Pseudomonas aeruginosa, Rhodococcus equi, Salmonella enterica, Serratia marcescens, Shigella dysenteriae, Shigella sonnei, Staphylococcus aureus, Staphylococcus epidermidis, Staphylococcus haemolyticus, Staphylococcus hominis, Staphylococcus saprophyticus, Stenotrophomonas maltophila, Streptococcus agalactiae, Streptococcus anginosus, Streptococcus bovis, Streptococcus constellatus, Streptococcus dysgalactiae, Streptococcus intermedins, Streptococcus mitis, Streptococcus mutans, Streptococcus oralis, Streptococcus pneumoniae, Streptococcus pyogenes, Streptococcus sanguinis, Streptococcus sobrinus, Streptomyces anulatus, Streptomyces somaliensis, Veillonella atypica, Veillonella denticariosi, Veillonella dispar, Veillonella parvula, Veillonella rogosae, Vibrio cholerae, Yersinia enterocolitica and Yersinia pestis.

As used herein, “sepsis” is defined as SIRS with a presumed or confirmed infectious process. Confirmation of infectious process can be determined using microbiological culture or isolation of the infectious agent. From an immunological perspective, sepsis may be seen as a systemic response to microorganisms or systemic infection.

“Systemic Inflammatory Response Syndrome (SIRS),” as used herein, refers to a clinical response arising from a non-specific insult with two or more of the following measureable clinical characteristics; a body temperature greater than 38° C. or less than 36° C., a heart rate greater than 90 beats per minute, a respiratory rate greater than 20 per minute, a white blood cell count (total leukocytes) greater than 12,000 per mm³ or less than 4,000 per mm³, or a band neutrophil percentage greater than 10%. From an immunological perspective, it may be seen as representing a systemic response to insult (e.g., major surgery) or systemic inflammation. As used herein, therefore, “infection-negative SIRS (inSIRS)” includes the clinical response noted above but in the absence of an identifiable infectious process.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which identical nucleic acid base (e.g., A, T, C, G) occurs in both sequence to yield the number of matched positions, dividing the number of matched positions by the total number of positions in the window of comparison (i.e., the window size), and multiplying the result by 100 to yield the percentage of sequence identity (% seq. identity).

As used herein, the term “single nucleotide polymorphism” or “SNP” refers to nucleotide sequence variations that occur when a single nucleotide (A, T, C or G) in the genome sequence is altered (such as via substitutions, addition or deletion). SNPs can occur in both coding (gene) and noncoding regions of the genome such as the genome of a prokaryotic or eukaryotic microorganism.

As used herein, the terms “treatment”, “treating” and the like, refer to obtaining a desired pharmacological and/or physiological effect. The effect may be prophylactic in terms of completely or partially preventing an infection, condition or symptom thereof and/or may be therapeutic in terms of a partial or complete cure for an infection, condition and/or adverse affect attributable to the infection or condition. “Treatment” as used herein covers any treatment of an infection or condition in a mammal (e.g., a human), and includes: (a) inhibiting the infection or condition, i.e., arresting its development; and (b) relieving the infection or condition, i.e., causing regression of the infection or condition.

### 2. Quantification of Bacteria Using Variation in SNPs and Gene Copy Number in 16S rRNA

The present invention provides methods for quantifying a bacterium in a sample, such as a biological sample from a subject. To this end, the present invention is based in part on the determination that SNPs and gene copy number of the 16S rRNA gene (and thus within the 16S rRNA molecule) that are specific to particular bacteria can be used to quantify individual species or strains of a bacterium.

Most particularly, the present invention provides methods for quantifying microorganisms, such as bacterial species selected from among: Aerococcus viridans; Acinetobacter calcoaceticus; Bacteroides fragilis; Cedecea lapagei; Citrobacter freundii; Cronobacter dublinensis; Enterobacter aerogenes; Enterobacter cloacae; Enterococcus cecorum; Enterococcus faecalis; Enterococcus faecium; Escherichia coli; Haemophilus influenzae; Klebsiella pneumoniae; Proteus mirabilis; Pseudomonas aeruginosa; Serratia marcescens; Shewanella putrefaciens; Staphylococcus aureus; Staphylococcus epidermidis; Streptococcus agalactiae; Streptococcus pneumoniae; and Streptococcus pyogenes. In one embodiment, the present invention provides methods for quantifying microorganisms, such as bacterial species selected from among: Acinetobacter calcoaceticus; Bacteroides fragilis; Enterobacter aerogenes; Enterobacter cloacae; Enterococcus faecalis; Enterococcus faecium; Escherichia coli; Haemophilus influenzae; Klebsiella pneumoniae; Proteus mirabilis; Pseudomonas aeruginosa; Serratia marcescens; Staphylococcus aureus; Staphylococcus epidermidis; Streptococcus agalactiae; Streptococcus pneumoniae; and Streptococcus pyogenes.

For example, the method for quantifying one of the bacterium listed in the above paragraph in a sample includes amplifying a target nucleic acid from a bacterial 16S rRNA gene of the microorganism in question from genetic material obtained from the sample to form an amplification product, measuring a quantity or concentration of the amplification product and calculating a quantity or concentration of the target nucleic acid in the sample by comparing the quantity or concentration of the amplification product with a reference level thereof.

Suitably, the target nucleic acid is amplified by PCR, such as quantitative PCR, semi-quantitative PCR, digital PCR and endpoint PCR or ligase chain reaction (LCR).

During PCR, the amount of DNA (e.g., the target nucleic acid) theoretically doubles with every cycle so as to generate the amplification product. After each cycle, the amount of DNA in the amplification product is approximately twice what it was before.

The absolute amount of the target nucleic in the sample is preferably determined using from a reference level, curve or value generated from one or a plurality of control samples or external standards. The control sample is usually very similar to the sample, such that the genetic material of the control sample contains the primer binding sites that should be identical to those in the sequence of the target nucleic acid. This ensures that the target nucleic acid in both the one or plurality of control samples and in the sample is amplified with equivalent efficiencies, which is typically required for quantification. In particular embodiments, the control samples comprise genetic material obtained from a known quantity or concentration of the bacterium. This may include live and/or killed bacteria of a known quantity or concentration thereof and/or genetic material extracted from a known quantity or concentration of the bacterium. In alternative embodiments, the control samples comprise a known quantity or concentration of synthetic oligonucleotides that include the target nucleic acid, for example said target nucleic acid may be substantially equivalent to a known quantity or concentration of the bacterium. Preferably, amplifying the target nucleic acid from the one or plurality of control samples is performed substantially simultaneously or in parallel with amplifying the target nucleic acid from the sample so as to avoid issues of inter-experimental (e.g., run to run, machine to machine) variability.

It is envisaged that the one or plurality of control samples can further comprise genetic material from one or a plurality of further bacteria, such as those hereinbefore described. To this end, the method of the present can be utilised to quantify at least two bacteria, at least three bacteria, at least four bacteria, at least five bacteria, at least six bacteria, at least seven bacteria, at least eight bacteria, at least nine bacteria, at least ten bacteria etc in the sample.

Furthermore, the bacterium within the sample may be as yet unknown and once identified, the amplification product can be compared to a reference level that corresponds to one or a plurality of control samples derived from the identified bacterium.

Using PCR techniques, such as quantitative PCR, a detectable label, such as a fluorescent label, is detected and measured in the PCR thermocycler, and its geometric increase corresponding to exponential increase of the product is used to determine the threshold cycle (CO in each reaction.

Suitably, the target nucleic acid of the bacterium from the sample and each of the control samples are amplified in separate tubes. A standard curve (plot of Cₜ value/crossing point against log of amount of standard) can then be generated using different dilutions of the control samples. The Cₜ value of the unknown samples is then compared with the result reference levels or standard curve of the appropriate bacterial class, genus or species, allowing calculation of the initial amount of the target nucleic acid in the sample. To generate a standard curve, suitably at least 2, at least 3, at least 4, at least 5 or at least 6 different amounts of the target nucleic acid should be quantified, and the amount of target nucleic acid within the sample should fall within the range of the standard curve.

The quantification methods described herein may further include the step of identifying the bacterium in the sample. As would be appreciated by the skilled person, this may be performed prior to, after or in conjunction with the present method. Moreover, and as noted earlier, identifying the bacterium can assist in selecting an appropriate reference level and gene copy number that corresponds to the particular bacterial class, genus and/or species identified.

In one embodiment, the step of identifying the bacterium comprises analysing the amplification product for the presence or absence of at least one SNP. As described herein, polymorphisms at nucleotide positions of the gene encoding 16S rRNA (and thus of the 16S rRNA molecule itself) that correspond to any one of positions 273, 378, 408, 412, 440, 488, 647 and 653 of the E. coli 16S rRNA gene as set forth in SEQ ID NO:1 can be used to identify bacterium within a sample, particularly including mammalian (e.g., human) pathogens (including the most commonly found bacterial species isolated by blood culture (Karlowsky et al. 2004)).

In one embodiment, the bacterium to be identified is selected from the group consisting of: Aerococcus viridans; Acinetobacter calcoaceticus; Bacteroides fragilis; Cedecea lapagei; Citrobacter freundii; Cronobacter dublinensis; Enterobacter aerogenes; Enterobacter cloacae; Enterococcus cecorum; Enterococcus faecalis; Enterococcus faecium; Escherichia coli; Haemophilus influenzae; Klebsiella pneumoniae; Proteus mirabilis; Pseudomonas aeruginosa; Serratia marcescens; Shewanella putrefaciens; Staphylococcus aureus; Staphylococcus epidermidis; Streptococcus agalactiae; Streptococcus pneumoniae; and Streptococcus pyogenes. In one embodiment, the bacterium to be identified is selected from the group consisting of: Acinetobacter calcoaceticus; Enterobacter aerogenes; Enterobacter cloacae; Enterococcus faecalis; Enterococcus faecium; Escherichia coli; Klebsiella pneumoniae; Proteus mirabilis; Pseudomonas aeruginosa; Serratia marcescens; Staphylococcus aureus; Staphylococcus epidermidis; Streptococcus agalactiae; Streptococcus pneumoniae; and Streptococcus pyogenes.

The general rules for identifying the above bacterial species within a sample using the above SNPs are hereinbefore described.

Any method known in the art to detect one or more SNPs can be used in the methods described herein to identify one or more bacterial species within a sample. In particular embodiments, the methods also facilitate in the narrowing down or, in some cases, confirming of one bacterial species over another. Numerous methods are known in the art for determining the nucleotide occurrence at a particular position corresponding to a single nucleotide polymorphism in a sample. The various tools for the detection of polymorphisms include, but are not limited to, DNA sequencing, scanning techniques, hybridization based techniques, extension based analysis, high-resolution melting analysis, incorporation based techniques, restriction enzyme based analysis and ligation based techniques.

The nucleic acid may be from a biological sample from a subject or from an environmental sample, such as an air, soil or water sample, a filtrate, a food or manufactured product, or swap from a surface, such as from a medical instrument or work place surface. The subject may be a human subject or non-human subject, such as a mammalian subject, such as primates, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., koalas, bears, wild cats, wild dogs, wolves, dingoes, foxes and the like). Biological samples from a subject may be from any part of the subject's body, including but not limited to bodily fluids such as blood, saliva, sputum, urine, cerebrospinal fluid, faeces, cells, tissue or biopsies. In other examples, the nucleic acid is obtained from cultured cells.

The nucleic acid that is analysed according to the methods of the present invention may be analysed while within the sample, or may first be extracted from the sample, e.g., isolated from the sample prior to analysis. Any method for isolating nucleic acid from a sample can be used in the methods of the present invention, and such methods are well known to those of skill in the art. The extracted nucleic acid can include DNA and/or RNA (including mRNA or rRNA). In some examples, a further step of reverse transcription can be included in the methods prior to analysis. Thus, the nucleic acid to be analysed can include the 16S rRNA gene, 16S rRNA, a DNA copy of the 16S rRNA or any combination thereof. The nucleic acid can also contain portions of the 16S rRNA gene, 16S rRNA, or a DNA copy of the 16S rRNA, providing the portions containing the nucleic acid positions that are being analysed for SNPs.

Such methods can utilise one or more oligonucleotide probes or primers, including, for example, an amplification primer pair, that selectively hybridize to a target polynucleotide, which contains one or more SNPs. Oligonucleotide probes useful in practicing a method of the invention can include, for example, an oligonucleotide that is complementary to and spans a portion of the target polynucleotide, including the position of the SNP, which the presence of a specific nucleotide at the polymorphic site (i.e., the SNP) is detected by the presence or absence of selective hybridization of the probe. Such a method can further include contacting the target polynucleotide and hybridized oligonucleotide with an endonuclease, and detecting the presence or absence of a cleavage product of the probe, depending on whether the nucleotide occurrence at the polymorphic site is complementary to the corresponding nucleotide of the probe.

Primers may be manufactured using any convenient method of synthesis. Examples of such methods may be found in “Protocols for Oligonucleotides and Analogues; Synthesis and Properties”, Methods in Molecular Biology Series, Volume 20, Ed. Sudhir Agrawal, Humana ISBN: 0-89603-247-7, 1993. The primers may also be labelled to facilitate detection.

Any method useful for the detection of SNPs can be used in the present invention, and many different methods are known in the art for SNP genotyping (for review see Syvanen, A. C. (2001); Kim, S. and Misra, A., (2007)). Such methodology may consist of the use of three steps in succession, including a “reaction” (e.g., hybridization, ligation, extension and cleavage) followed by “separation” (e.g., solid phase microtitre plates, microparticles or arrays, gel electrophoresis, solution-phase homogenous or semi-homogenous). No single ideal SNP genotyping method exists for all applications, and it is well within the skill of a skilled artisan to determine the most appropriate method given the various parameters, such as sample size and number of SNPs to be analysed.

Example technologies that particularly lend themselves to clinical use and that rely on querying small numbers of SNPs, are fast, sensitive (through amplification of nucleic acid in the sample), one-step, output measured in real-time, able to be multiplexed and automated, comparatively inexpensive, and accurate include, but are not limited to, TaqMan® assays (5′ nuclease assay, Applied Biosystems), high-resolution melt analysis, molecular beacon probes such as LUX® (Invitrogen) or Scorpion® probes (Sigma Aldrich), and Template Directed Dye Incorporation (TDI, Perkin Elmer).

For example, TaqMan® (Applied Biosystems) uses a combination of hybridization with allele-specific probes, solution phase homogenous, and fluorescence resonance energy transfer. The TaqMan® assay relies on forward and reverse primers and Taq DNA polymerase to amplify nucleic acid in conjunction with 5′-nuclease activity of Taq DNA polymerase to degrade a labelled probe designed to bind across the SNP site(s). Reaction, separation and detection can all be performed at the same time and results read in real-time as the reaction proceeds. While such an approach does not lend itself to analysing large numbers of SNPs simultaneously it is particularly suitable for querying small numbers of SNPs quickly, sensitively and accurately at a reasonable cost.

Although some methods may be more suitable than others, any method known in the art to detect one or more SNPs can be used in the methods described herein to classify and/or identify bacteria and/or bacterium in a sample.

In one preferred embodiment, however, detecting the presence or the absence of the at least one SNP comprises the use of high resolution melt (HRM) analysis. To this end, utilisation of high resolution melt analysis in the present method advantageously allows for both quantification and identification of the bacterium to be achieved from a single amplification step of the target nucleic acid of the bacterial 16S rRNA gene.

HRM is based upon the accurate monitoring of changes in fluorescence as a PCR product (i.e., amplicon) stained with an intercalating fluorescent dye is heated through its melting temperature (Tₘ). In contrast to traditional melting, the information in HRM analysis is contained in the shape of the melting curve, rather than just the calculated Tₘ, so HRM may be considered a form of spectroscopy. HRM analysis is a single step and closed tube method, the amplification and melting can be run as a single protocol on a real-time PCR machine.

In embodiments of the present invention, the methods utilise an amplification primer pair that selectively hybridize to a target polynucleotide containing one or more of the SNPs as described herein. The amplification reaction mixture contains the fluorescent dye, which is incorporated into the resulting amplicon.

The resulting amplicon is then subjected to HRM with incremental increases in temperature (i.e., 0.01-0.5° C.) ranging from about 50° C. to about 95° C. At some point during this process, the melting temperature of the amplicon is reached and the two strands of DNA separate or “melt” apart.

The HRM is monitored in real-time using the fluorescent dye incorporated into the amplicon. The level of fluorescence of the dye is monitored as the temperature increases with the fluorescence reducing as the amount of double stranded DNA reduces. Changes in fluorescence and temperature can be plotted in a graph known as a melt curve.

As a skilled addressee will understand, the Tₘ of the amplicon at which the two DNA strands separate is predictable, being dependent on the sequence of the nucleotide bases forming the amplicon. Accordingly, it is possible to differentiate between amplicons including an amplicon containing a polymorphism (i.e., a SNP or SNPs) as the melt curves will appear different. Indeed, in some embodiments, it is possible to differentiate between amplicons containing the same polymorphism based on differences in the surrounding DNA sequences.

HRM curves can be discriminated from one another by many different strategies. For example, in many cases, HRM curves can be discriminated on the basis of obvious differences in curve shape and/or on the basis of Tₘ with a difference of 0.2° C. being regarded as significant. In other cases, a difference graph analysis can be used in which a defined curve is used as a baseline with other normalised curves being plotted in relation to the baseline (see Price, E. P. et al. 2007). In yet other cases, a difference graph-based method can be used involving deriving the 3rd and 97th centiles from the mean±1.96 standard deviations for the fluorescence at every temperature (see Andersson, P. et al., 2009; and Merchant-Patel, S. et al. 2008).

### 3. Kits

The specification explains how various SNPs and gene copy numbers of the 16S rRNA gene can be used as ‘tools’ for quantifying a bacterium in a sample. These findings of the present invention enable the inventors to develop gene/allele-based and gene product-based probes, tools, reagents, methods and assays for quantifying a bacterium in a sample.

In this regard, the kit may comprise one or a plurality of control samples, such as those described herein, for determination of a reference level, value or curve, and/or information about obtaining a reference level, value or curve. In some embodiments, the kit may further comprise instructions on use of the kits for quantifying a bacterium, as described herein.

In one embodiment, the kit or assay comprises at least one isolated probe, tool or reagent that is capable of identifying, partially identifying, or classifying at least one bacteria in a sample, wherein the probe, tool or reagent is capable of binding, detecting or identifying the presence or absence of at least one single nucleotide polymorphism (SNP), such as those described herein, in at least a portion of a bacterial 16S rRNA gene. In particular embodiments, the at least one SNP is at a position corresponding to at least one of positions 273, 378, 408, 412, 440, 488, 647, 653, 737, 755, 762 and 776 of the 16S rRNA gene set forth in SEQ ID NO: 1.

In one embodiment, said at least one isolated probe, tool or reagent is capable of discriminating between a sample that comprises at least one bacterium and a sample that does not comprise at least one bacterium.

In particular embodiments, the kit or assay is or comprises an array or microarray of oligonucleotide probes for identifying the bacterium and optionally one or a plurality of further bacteria in a sample, said probes comprising oligonucleotides which hybridize to at least one SNP in a 16S rRNA gene in the sample as broadly described above.

In another embodiment, the kit or assay is or comprises a biochip comprising a solid substrate and at least one oligonucleotide probe for identifying the bacterium and optionally one or a plurality of further bacteria in a sample, said at least one probe comprising an oligonucleotide which hybridizes to at least one SNP in a 16S rRNA gene in the sample as broadly described above.

All the essential materials and reagents required for amplifying the target nucleic acid in the sample and/or the one or plurality of control samples and/or detecting one or more SNPs in the 16S rRNA gene according to the invention may be assembled together in a kit. The kits may also optionally include appropriate reagents for detection of labels, positive and negative controls, fluorescent dyes, washing solutions, blotting membranes, microtitre plates, dilution buffers and the like. For example, a nucleic acid-based detection kit for the identification of polymorphisms may include one or more of the following: (i) nucleic acid from a Gram-positive cell and/or Gram-negative cell (which may be used as a positive control); and (ii) a primer and/or probe that specifically hybridizes to at least a portion of the 16S rRNA gene containing the SNP position(s) to be analysed, and optionally one or more other markers, at or around the suspected SNP site. Also included may be enzymes suitable for amplifying nucleic acids including various polymerases (Reverse Transcriptase, Taq, Sequenase™ DNA ligase etc. depending on the nucleic acid amplification technique employed), deoxynucleotides and buffers to provide the necessary reaction mixture for amplification. Such kits also generally will comprise, in suitable means, distinct containers for each individual reagent and enzyme as well as for each primer or probe. The kit can also feature various devices and reagents for performing one of the assays described herein; and/or printed instructions for using the kit to identify the presence of a SNP as defined herein.

In some embodiments, the methods described generally herein are performed, at least in part, by a processing system, such as a suitably programmed computer system. A stand-alone computer, with the microprocessor executing applications software allowing the above-described methods to be performed, may be used. Alternatively, the methods can be performed, at least in part, by one or more processing systems operating as part of a distributed architecture. For example, a processing system can be used to measure a quantity or concentration of the amplification product, calculate a quantity or concentration of the target nucleic acid in the sample by comparing the quantity or concentration of the amplification product with a reference level thereof and/or quantify the bacterium by determining a genome copy number from the quantity or concentration of the target nucleic acid in the sample. A processing system also can be used to identify the bacterium on the basis of detection of one or more SNPs. In some examples, commands inputted to the processing system by a user may assist the processing system in making these determinations.

In one example, a processing system includes at least one microprocessor, a memory, an input/output device, such as a keyboard and/or display, and an external interface, interconnected via a bus. The external interface can be utilised for connecting the processing system to peripheral devices, such as a communications network, database, or storage devices. The microprocessor can execute instructions in the form of applications software stored in the memory to allow the SNP detection and/or bacterium quantification process to be performed, as well as to perform any other required processes, such as communicating with the computer systems. The application software may include one or more software modules, and may be executed in a suitable execution environment, such as an operating system environment, or the like.

3.1 Primers, Probes, Kits and Processing Systems for the 16S rRNA Gene Gene

Non-limiting examples of primers and probes that are useful for the methods of the present invention, in which SNPs in the 16S rRNA of bacterial species at positions corresponding to positions 273, 378, 408, 412, 440, 488, 647 and/or 653 of the 16S rRNA gene set forth in SEQ ID NO:1 are analysed, are set out below.

For example, to detect SNPs at position 273 an exemplary forward primer includes CCTCTTGCCATCGGATGTG (SEQ ID NO:16) and exemplary reverse primers include CCAGTGTGGCTGGTCATCCT (SEQ ID NO:17), CGATCCGAAAACCTTCTTCACT (SEQ ID NO:20), CTATGCATCGTTGCCTTGGTAA (SEQ ID NO:22), TGATGTACTATTAACACATCAACCTTCCT (SEQ ID NO:26), AACGCTCGGATCTTCCGTATTA (SEQ ID NO:27), CGCTCGCCACCTACGTATTAC (SEQ ID NO:28), CGTAGTTAGCCGTCCCTTTCTG (SEQ ID NO:30), GGAATTCTACCCCCCTCTACGA (SEQ ID NO:34), and GGAATTCTACCCCCCTCTACAAG (SEQ ID NO:35).

To detect SNPs at position 378, exemplary forward primers include CCTCTTGCCATCGGATGTG (SEQ ID NO:16), CCTACGGGAGGCAGCAGTAG (SEQ ID NO:18), GGGAGGCAGCAGTAGGGAAT (SEQ ID NO:19) and TCCTACGGGAGGCAGCAGTAGGG (SEQ ID NO:48); and exemplary reverse primers include CGATCCGAAAACCTTCTTCACT (SEQ ID NO:20), CTATGCATCGTTGCCTTGGTAA (SEQ ID NO:22), TGATGTACTATTAACACATCAACCTTCCT (SEQ ID NO:26), AACGCTCGGATCTTCCGTATTA (SEQ ID NO:27), CGCTCGCCACCTACGTATTAC (SEQ ID NO:28), CGTAGTTAGCCGTCCCTTTCTG (SEQ ID NO:30), GGAATTCTACCCCCCTCTACGA (SEQ ID NO:34), GGAATTCTACCCCCCTCTACAAG (SEQ ID NO:35) and CCGCTACACATGGAATTCCAC (SEQ ID NO:49).

To detect SNPs at position 408, exemplary forward primers include GACTCCTACGGGAGGCAGCAGTGGG (SEQ ID NO:50) and exemplary reverse primers include GGTATTAACTTACTGCCCTTCCTCCC (SEQ ID NO:51).

To detect SNPs at position 412, exemplary forward primers include CCTCTTGCCATCGGATGTG (SEQ ID NO:16), CCTACGGGAGGCAGCAGTAG (SEQ ID NO:18), GGGAGGCAGCAGTAGGGAAT (SEQ ID NO:19), and AAGACGGTCTTGCTGTCACTTATAGA (SEQ ID NO:21); and exemplary reverse primers include CTATGCATCGTTGCCTTGGTAA (SEQ ID NO:22), TGATGTACTATTAACACATCAACCTTCCT (SEQ ID NO:26), AACGCTCGGATCTTCCGTATTA (SEQ ID NO:27), CGCTCGCCACCTACGTATTAC (SEQ ID NO:28), CGTAGTTAGCCGTCCCTTTCTG (SEQ ID NO:30), GGAATTCTACCCCCCTCTACGA (SEQ ID NO:34), and GGAATTCTACCCCCCTCTACAAG (SEQ ID NO:35).

To detect SNPs at position 440, exemplary forward primers include CCTCTTGCCATCGGATGTG (SEQ ID NO:16), CCTACGGGAGGCAGCAGTAG (SEQ ID NO:18), GGGAGGCAGCAGTAGGGAAT (SEQ ID NO:19), AAGACGGTCTTGCTGTCACTTATAGA (SEQ ID NO:21), TGCCGCGTGAATGAAGAA (SEQ ID NO:23), GCGTGAAGGATGAAGGCTCTA (SEQ ID NO:24), and TGATGAAGGTTTTCGGATCGT (SEQ ID NO:25); and exemplary reverse primers include TGATGTACTATTAACACATCAACCTTCCT (SEQ ID NO:26), AACGCTCGGATCTTCCGTATTA (SEQ ID NO:27), CGCTCGCCACCTACGTATTAC (SEQ ID NO:28), CGTAGTTAGCCGTCCCTTTCTG (SEQ ID NO:30), GGAATTCTACCCCCCTCTACGA (SEQ ID NO:34), and GGAATTCTACCCCCCTCTACAAG (SEQ ID NO:35).

To detect SNPs at position 488, exemplary forward primers include CCTCTTGCCATCGGATGTG (SEQ ID NO:16), CCTACGGGAGGCAGCAGTAG (SEQ ID NO:18), GGGAGGCAGCAGTAGGGAAT (SEQ ID NO:19), AAGACGGTCTTGCTGTCACTTATAGA (SEQ ID NO:21), TGCCGCGTGAATGAAGAA (SEQ ID NO:23), GCGTGAAGGATGAAGGCTCTA (SEQ ID NO:24), TGATGAAGGTTTTCGGATCGT (SEQ ID NO:25), and GTTGTAAGAGAAGAACGAGTGTGAGAGT (SEQ ID NO:29); and exemplary reverse primers include CGTAGTTAGCCGTCCCTTTCTG (SEQ ID NO:30), GGAATTCTACCCCCCTCTACGA (SEQ ID NO:34), and GGAATTCTACCCCCCTCTACAAG (SEQ ID NO:35).

To detect SNPs at positions 647 and/or 653, exemplary forward primers include CCTCTTGCCATCGGATGTG (SEQ ID NO:16), CCTACGGGAGGCAGCAGTAG (SEQ ID NO:18), GGGAGGCAGCAGTAGGGAAT (SEQ ID NO:19), AAGACGGTCTTGCTGTCACTTATAGA (SEQ ID NO:21), TGCCGCGTGAATGAAGAA (SEQ ID NO:23), GCGTGAAGGATGAAGGCTCTA (SEQ ID NO:24), TGATGAAGGTTTTCGGATCGT (SEQ ID NO:25), GTTGTAAGAGAAGAACGAGTGTGAGAGT (SEQ ID NO:29), GCGGTTTGTTAAGTCAGATGTGAA (SEQ ID NO:31), GGTCTGTCAAGTCGGATGTGAA (SEQ ID NO:32), and TCAACCTGGGAACTCATTCGA (SEQ ID NO:33); and exemplary reverse primers include GGAATTCTACCCCCCTCTACGA (SEQ ID NO:34), and GGAATTCTACCCCCCTCTACAAG (SEQ ID NO:35).

Similarly, non-limiting examples of primers and probes that are useful for the methods of the present invention, in which SNPs in the 16S rRNA gene or 16S rRNA of bacterial species at positions corresponding to positions corresponding to positions 746, 764, 771, or 785 of the 16S rRNA gene as set forth in SEQ ID NO:38 (or positions 737, 755, 762, or 776 of the 16S rRNA gene as set forth in SEQ ID NO:1) are analysed, include those described in Table 7.

### 4. Applications of the Methods of the Present Invention

The methods of the present invention are useful for quantifying one or more bacteria in a sample, such as a sample from a subject or an environmental sample such as a soil or water sample or a sample taken from the surface of equipment or instruments (e.g. medical or surgical instruments) or a work surface. Such quantification can then be used to determine, for example, a course of treatment to remove, eradicate or reduce the number of bacteria. Any two or more of the methods of the present invention can be combined. For example, bacteria in a biological sample from a subject with a bacterial infection can be quantified so as to determine a prognosis for that subject, as well as how to treat the infection.

Subjects with infections or suspected infections often present to clinicians in clinics, emergency rooms, general wards and intensive care units. Such patients often have non-diagnostic clinical signs of abnormal temperature, increased heart and respiratory rates and abnormal white cells counts. A clinician must decide whether the patient has an infection or not, the severity of the infection, whether to admit the patient to hospital (if not already in hospital), the source of infection, whether to use antibiotics, and if so, the type, route and dose of antibiotics. The presence of an infection in a patient has most typically been assessed by taking a sample from the patient and growing an organism in culture broth. Once an organism has grown it can be Gram stained and identified. However, such methods do not accurately quantify the level of bacterial infection in the subject and hence fail to provide a prognostic outlook for the patient. Without quantifying the bacteria, a clinician must rely on his or her clinical judgment so as to determine a therapeutic regime for the subject.

Thus, the methods of the present invention are particularly useful in assisting clinicians in determining a prognosis, an appropriate course of treatment and treatment efficacy based on the quantification, and optionally identification, of the bacteria causing the infection. In particular embodiments, the method of the present invention can be used to determine antibiotic resistance in a subject.

The methods of the present invention also can be performed in a time-efficient manner, so that the results are available to the clinician within hours rather than days. Such attributes allow a clinician to sensitively quantify levels of a bacterium in a subject and to make an informed decision on treatment. These improvements can result in a reduced number of patients admitted to hospital unnecessarily, sensitive detection of bacteria, severity of infection, reduced use of broad-spectrum antibiotics/medicines, reduced patient time on broad spectrum antibiotics, reduced toxicity from antibiotics/medicines and reduced development of resistance to medicines (especially antibiotic resistance).

Based on the results of the methods of the present invention, the subject can be appropriately managed and administered therapy where required. For example, the management of a bacterial infection can include, for example, administration of therapeutic agents such as a therapeutically effective course of antibiotics.

Typically, therapeutic agents will be administered in pharmaceutical (or veterinary if the subject is a non-human subject) compositions together with a pharmaceutically acceptable carrier and in an effective amount to achieve their intended purpose. The dose of active compounds administered to a subject should be sufficient to achieve a beneficial response in the subject over time such as a reduction in, or relief from, the symptoms of the infection, and/or the reduction or elimination of the bacteria from the subject. The quantity of the pharmaceutically active compounds(s) to be administered may depend on the subject to be treated inclusive of the age, sex, weight and general health condition thereof. In this regard, precise amounts of the active compound(s) for administration will depend on the judgment of the practitioner. In determining the effective amount of the active compound(s) to be administered in the treatment or prevention of the bacterial infection, the practitioner may evaluate severity of infection, and severity of any symptom associated with the infection including, inflammation, blood pressure anomaly, tachycardia, tachypnoea, fever, chills, vomiting, diarrhoea, skin rash, headaches, confusion, muscle aches and seizures. In any event, those of skill in the art may readily determine suitable dosages of the therapeutic agents and suitable treatment regimens without undue experimentation.

The therapeutic agents may be administered in concert with adjunctive (palliative) therapies to increase oxygen supply to major organs, increase blood flow to major organs and/or to reduce the inflammatory response. Illustrative examples of such adjunctive therapies include non-steroidal-anti inflammatory drugs (NSAIDs), intravenous saline and oxygen.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims (if any) appropriately interpreted by those skilled in the art.

All computer programs, algorithms, patents, accession numbers and scientific literature referred to herein is incorporated in their entirety herein by reference.

In order that the invention may be readily understood and put into practical effect, particular preferred embodiments will now be described by way of the following non-limiting examples.

### EXAMPLES

### Example 1

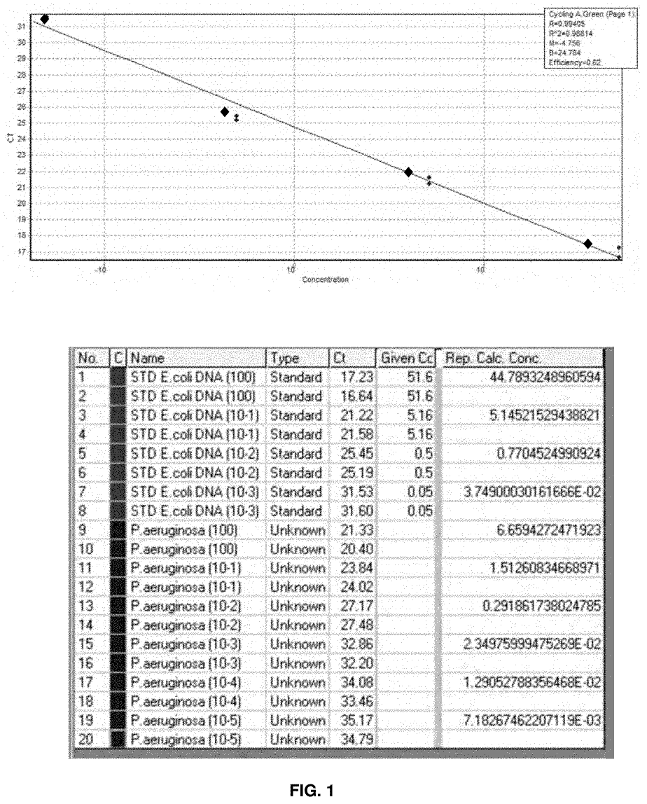

The present method (InfectID®) uses the core principles of real time PCR (qPCR) and High Resolution Melt (HRM) to accurately quantify and identify the pathogens within a given patient sample. The process involves taking blood directly from patients, extracting pathogen DNA, amplifying it and measuring the HRM curve to differentiate between species. Like other molecular identification systems, the InfectID® process amplifies DNA to detectable levels. However, the key difference is the DNA is amplified alongside samples of control DNA which is present in known quantities. Because these quantities form a gradient, a line of best fit can be drawn correlating DNA (ng) to volume (ul). An example is shown in FIG. 1.

It is only after the DNA quantity is measured during amplification it is broken apart for HRM curve analysis and speciation. Once the amount of DNA is measured in nanograms (ng) the number of bacterial cells or genome copies can be calculated using the genome copy number equation.

The gene copy number is the number of copies of a gene in the genotype of a given pathogen. InfectID® identifies and amplifies a region of the 16S gene and because bacteria only have one chromosome measuring the number of 16S molecules in a sample directly correlates to the number of bacterial cells. Different pathogens have different numbers of 16S genes in their chromosome, for example Staphylococcus aureus has 5 gene copies of 16S while Klebsiella pneumoniae has 8 copies. By accounting for this variance and dividing the total number of gene copies by the number of 16S copies in the genome, total bacterial numbers can be calculated using known values, such as by the algorithm below.

$$Number of copies ( molecules ) = \frac{X ng \times 6.0221 \times {10}^{23} molecules / mole}{( N \times 660 g / mole ) \times 1 \times {10}^{9} ng / g}$$

### Where:

X=amount of amplicon (ng)

N=length of dsRNA amplicon

660 g/mole=average mass of 1 bp dsRNA

### Results

A summary of the sensitivity of InfectID® is shown in Table 9 below. These cells values were quantified off DNA data from the Rotorgene which was then used in the genome copy number formula above.

TABLE 9

| | Cells/0.25 | Cells/0.5 |
|---|---|---|
| | mL Blood | mL Blood |
| Staphylococcus aureus | 262 | 394 |
| Staphylococcus epidermidis | 291 | 296 |
| Streptococcus agalactiae | 122 | 490 |
| Serratiamarcescens | 388 | 129 |
| Proteusmirabilis | 7171 | 325 |
| Streptococcus pneumoniae | 1590 | 2270 |
| Enterobacter cloacae | 261 | 261 |
| Pseudomonas aeruginosa | 370 | 370 |
| Streptococcus pyogenes | 33 | 333 |
| Enterococcus faecalis | 1762 | 1762 |
| Enterococcus faecium | 571 | 114 |
| Bacteroides fragilis | 58 | 175 |
| Escherichia coli | 100 | 260 |
| Klebsiella pneumoniae | 873 | 283 |
| Elaemophilus influenzae | 50 | 843 |

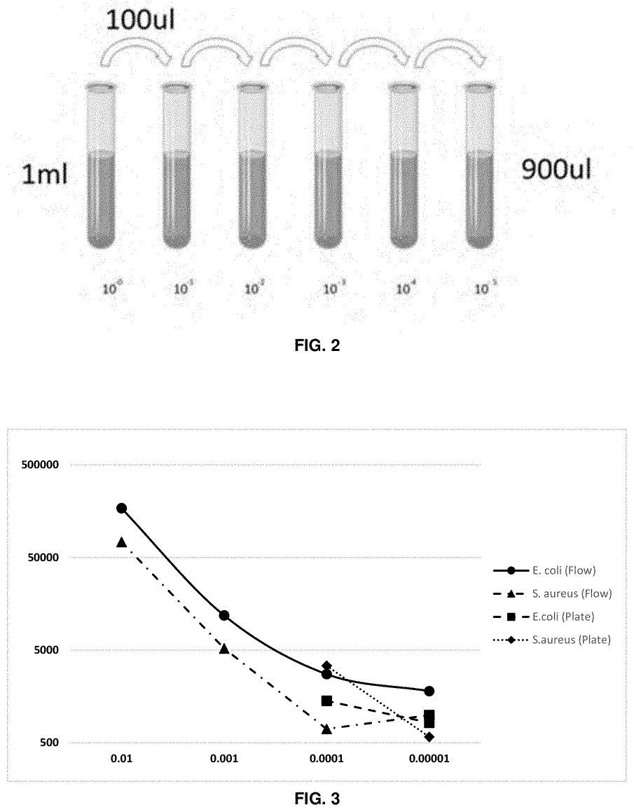

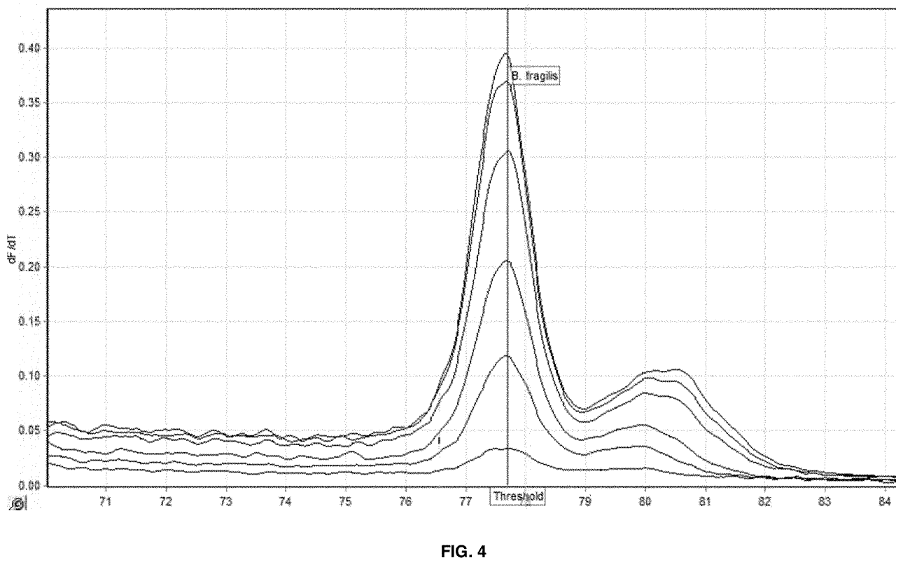

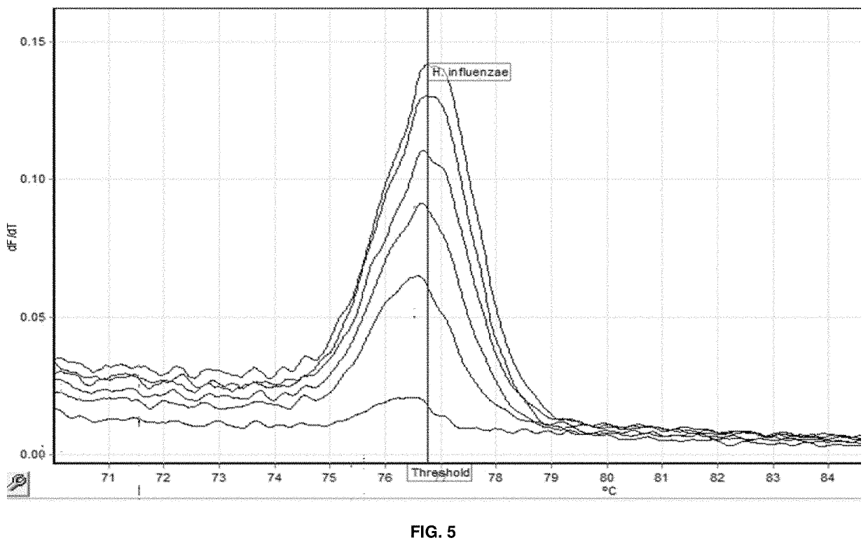

A major hurdle for InfectID® is breaking the current nomenclature and helping clinicians and pathologists understand how sensitive genome copy number is and how this relates to CFU and total cells. To do this, the present inventors ran multiple experiments to quantify the number of pathogenic cells in a given sample. Colonies of a number of species of interest were grown for a period of between 12-16 hours depending on the species before a single colony of known pathogenic cell concentration was spiked into a sample. As an example growth of E. coli over a 12 hour period results in a colony of between 10⁷-10⁸ cells. Since E. coli is the fastest growing bacteria all other colonies would have less cells to begin with which further shows the strength of InfectID® sensitivity. The blood and spiking was done with a single colony diluted down by a factor of 10 in serial dilution as shown in FIG. 2.

When running experiments through the InfectID system, PBS and blood were spiked with pathogens of interest to test sensitivity and specificity. During this time a sample of the spiked component was plated out onto traditional culture plate assay. The resulting CFU counts were compared to the results for InfectID, as illustrated in Table 10 below.

TABLE 10
Comparison of InfectID ® genome copies/3
50 μL and CFU/mE

| Bacterial | Genome copies/ | PBS | Blood |
|---|---|---|---|
| species | 350 uL | CFU/mL | CFU/mL |
| E. coli | 5.29 × 10² | 2.80 × 10⁵ | 2.96 × 10⁴ |
| E. cloacae | 9.95 × 10² | 1.89 × 10⁵ | 2.07 × 10⁴ |
| S. marcescens | 1.14 × 10³ | 7.35 × 10⁵ | 1.62 × 10⁴ |
| K. pneumoniae | 1.22 × 10² | 1.16 × 10⁵ | 3.83 × 10⁴ |
| P. mirabilis | 6.85 × 10² | 7.70 × 10⁵ | 4.9 × 10⁴ |
| S. aureus | 2.2 × 10³ | 7.30 × 10⁵ | 2.88 × 10⁴ |
| S. epidermidis | 7.13 × 10³ | 3.30 × 10⁵ | 1.46 × 10⁴ |
| E. faecalis | 7.68 × 10² | 1.08 × 10⁵ | 1.02 × 10⁴ |
| E. faecium | 1.07 × 10³ | 5.30 × 10⁵ | 2.35 × 10⁴ |
| S. agalactiae | 8.54 × 10² | 9.15 × 10⁵ | 3.17 × 10⁴ |
| S. pyogenes | 9.92 × 10² | 4.40 × 10⁵ | 1.9 × 10¹ |
| S. pneumoniae | 1.2 × 10⁴ | 6.20 × 10⁵ | 1.74 × 10² |
| P. aeruginosa | 5.83 × 10⁴ | 2.70 × 10⁵ | 7.8 × 10³ |
| H. influenzae | 5.57 × 10² | 4.20 × 10⁵ | 2.15 × 10⁰ |
| B. fragilis | 3.64 × 10² | 4.40 × 10⁵ | 2.86 × 10² |

Flow cytometry is the direct counting of individual molecules based on size and fluorescence. In this context bacteria cells were stained with fluorescent dye and individual bacteria were counted as they were interrogated one cell at a time through a laser.

The results of the quantitation experiments are shown in FIG. 3. Due to the limited ability to count individual colonies at lower dilutions results were recorded only at higher dilutions for the plate counts. The total number of cells shown below relate to the volume of the sample used in InfectID® which is 350 μL. Table 11 shows data illustrated in FIG. 3 and is provided below.

TABLE 11
Results of comparative flow cytometry and plate
count data for E. coli and S. aureus

| | | 10⁻² | 10⁻³ | 10⁻⁴ | 10⁻⁵ |
|---|---|---|---|---|---|
| E Coli | Flow | 169852 | 11839 | 2744 | 1806 |
| | McFarland | n/a | n/a | 1414 | 823 |
| | Plate | | | | |
| S. Aureus | Flow | 73395 | 5212 | 704 | 944 |
| | McFarland | n/a | n/a | 3391 | 579 |
| | Plate | | | | |

### Example 2

### Background

The methods of the present invention may assist in reducing the impact of sepsis, a global public health threat that claims millions of lives and costs billions every year.

Sepsis is a potentially life-threatening reaction to an infection. Identifying the pathogen responsible for the infection and subsequently the most effective course of treatment takes many hours, even days. In the meantime, clinicians can only guess which antibiotics are necessary to arrest this fast-moving and frequently fatal condition. Consequently, while waiting for test results, doctors typically administer broad-spectrum antibiotics, which are often inappropriate and therefore ineffective.

InfectID® detects sepsis-causing pathogens directly from blood (no culturing required). InfectID® is based on testing a small number of SNPs to separate and identify multiple pathogens of interest. To do this, InfectID® uses real-time PCR followed by high-resolution melt-curve analysis (MCA) to delineate the species of the pathogen directly in blood.

The extremely low levels of bacteria (1 to 100 cells/mL) found in a septic patient's blood increase the difficult challenges of rapid diagnosis (Reimer et al., 1997). Two further studies have also indicated that at the time the patient begins to show clinical symptoms of sepsis, the concentration of bacteria present in blood is very low (1 to 100 CFU/mL in adults (Yagupsky et al., 1990) and <10 CFU/mL in neonates (Reier-Nilsen et al., 2009). The current gold standard for diagnosis of blood stream infections and sepsis is blood culture, and a severe limitation of the gold standard is that the concentrations of bacteria in the suspension typically has to rise to 108 CFU/mL before they can be detected (Smith et al., 2008).

In a series of 20 adults with bacteremia and septic shock, Hall and Gold (1955) found that all six patients with >100 CFU/mL of blood died while only 41% of those with lesser bacteremia died. Although the number of microorganisms in blood showed a moderate correlation with the occurrence and severity of septic shock, some patients with bacterial counts as high as 300 CFU/mL of blood did not suffer shock. In another study, Weil and Spink (1958) found a correlation between magnitude of bacteremia in patients with gram-negative sepsis and fatal outcome. The mortality rate for eight patients with 5 CFU were isolated from patients' blood, mortality increased to 84%.

It has been demonstrated that InfectID® is a highly specific and sensitive direct-from-blood diagnostic test to identify the top 20 bacterial and 5 yeast pathogens most commonly associated with blood stream infections and sepsis. The utility of InfectID® as a tool to determine severity of blood stream infections and sepsis could have a significant impact on the antibiotic treatment of patients.

The aim of the present Example was to determine the limit of detecting bacterial species that cause sepsis in spiked EDTA blood using InfectID®.

### Methods

1. In total, 20 of the most prevalent bacterial species were tested in this study.
2. The 20 American Type Culture Collection (ATCC) bacterial species were cultured in Brain Heart Infusion agar overnight at 37° C.
3. A single bacterial colony from each bacterial species was used to spike 1 mL of EDTA blood, which was then serially diluted 1:9 fold to generate a dilution series of spiked blood.
4. DNA was extracted from the spiked blood samples using the Roche MagNApure system.
5. In addition, 1 mL of EDTA blood obtained from patients admitted to the Royal Brisbane & Women's Hospital, was also subjected to DNA extraction using the Roche Magnapure system.
6. InfectID® SNP primers (Integrated DNA Technologies, Australia) were designed to amplify the regions encompassing the highly discriminatory SNPs. PCR products sizes ranged from 79 bp to 96 bp.
7. Quantitative Real-Time PCR (qPCR) was used to determine the concentration of the 20 bacterial species in spiked blood as well as in patient blood. The qPCR process was: One microliter of extracted DNA (1 to 3 ng) was added to 19 μl of reaction mastermix containing 10 μl of the 2× Type-it-HRM Mastermix (Qiagen, Australia) and 8 pmol of each primer. Temperature cycling for these reactions were as follows: 50° C. for 2 min, 95° C. for 2 min, followed by 40 cycles of 95° C. for 15 s, 52° C. for 20 s, and 72° C. for 35 s, Hold at 72° C. for 2 min, Hold at 50° C. for 20 s, (RotorGeneQ, Qiagen, Australia).
8. The RotorGeneQ (Qiagen, Australia) software enables the user to visualize HRM data in multiple ways. The normalized raw melt curve depicts the decreasing fluorescence vs increasing temperature, and the difference curve, which displays a user-defined curve as the baseline (i.e. the x-axis), and depicts other normalized curves in relation to that baseline.

### Results

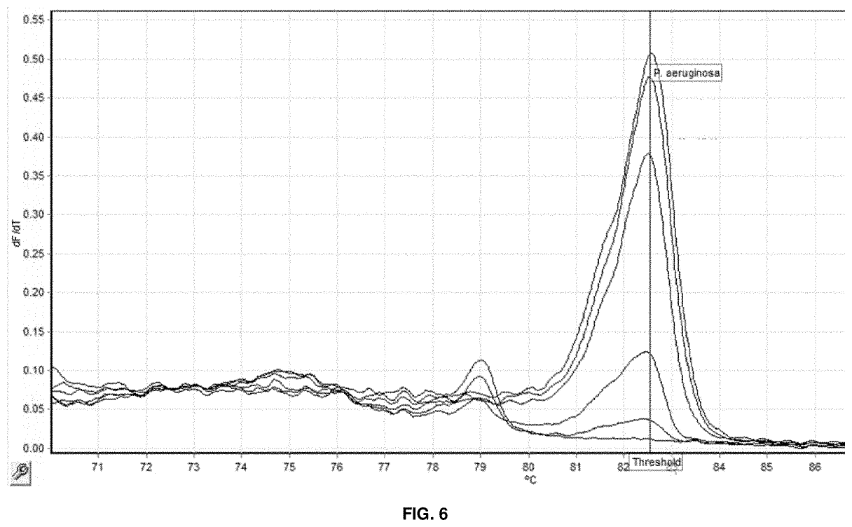

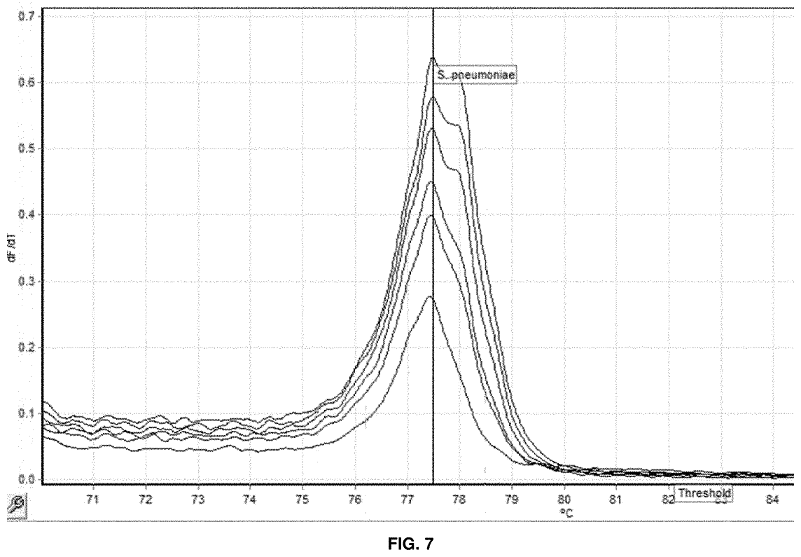

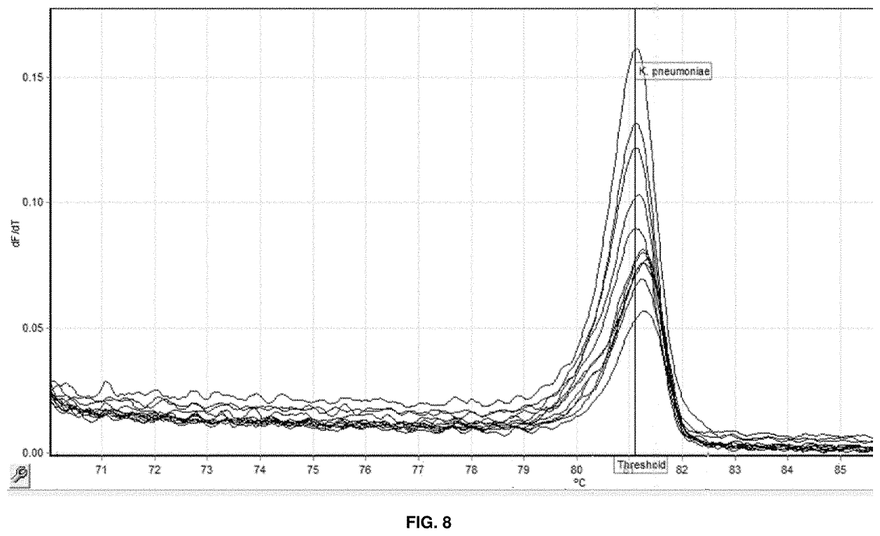

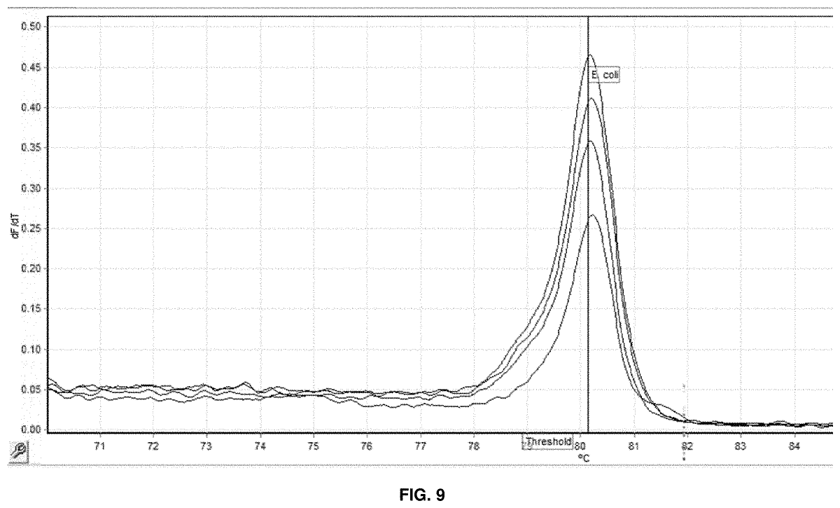

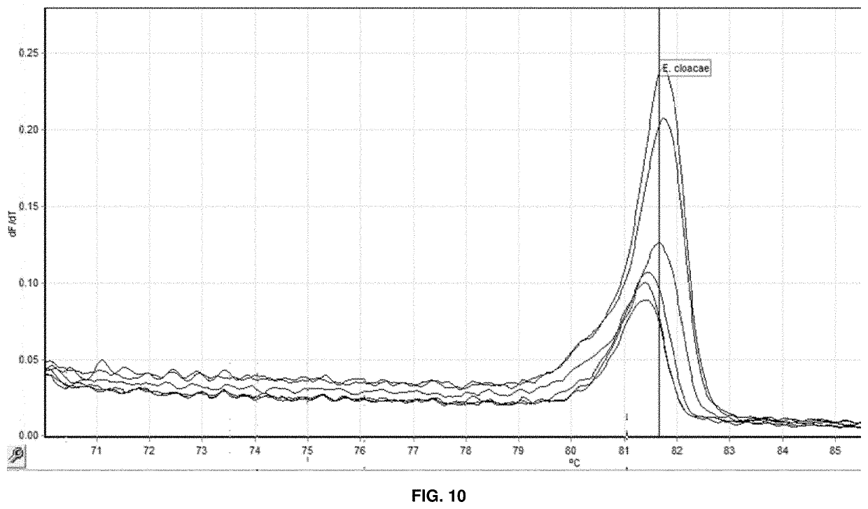

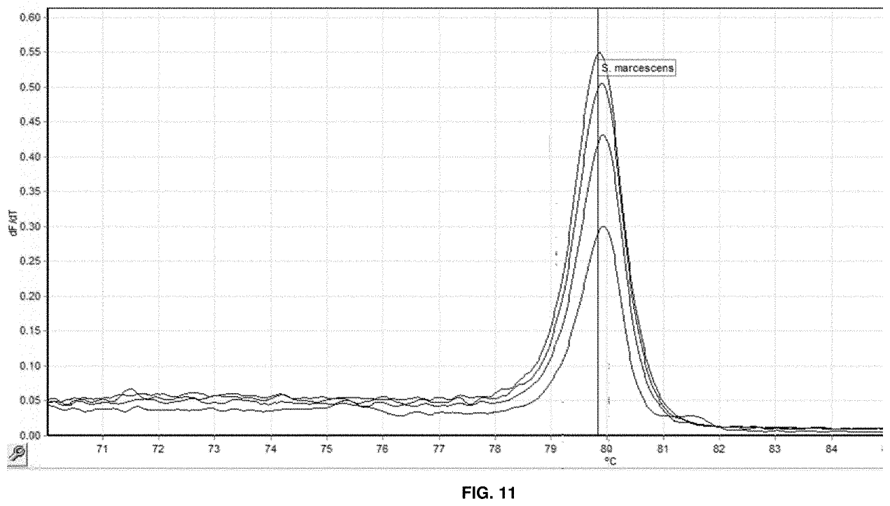

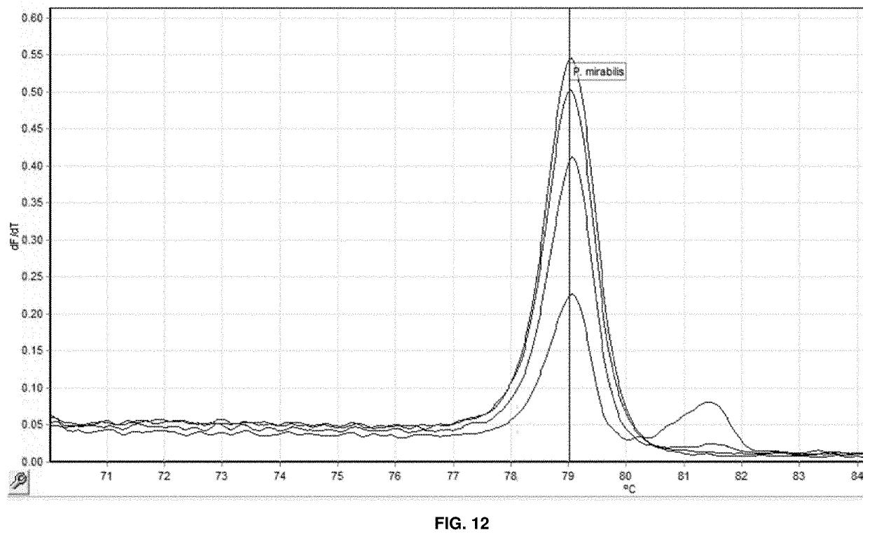

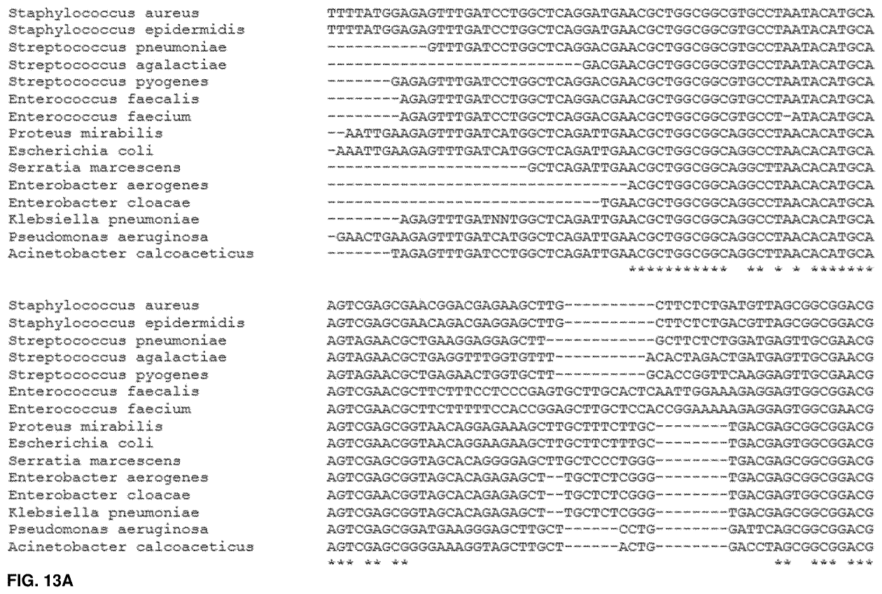

The limits of detection for the various bacterial species tested are shown in Table 13 and the melt curve analysis for this data is shown in FIG. 6.

Data with respect to the testing and bacterial quantification of patient samples is illustrated in Table 14.

TABLE 13
Limit of Detection (LOD) values for the control
samples of the bacterial species
assayed-Bacterial quantitation in spiked blood.

| | | | Genome |
|---|---|---|---|
| SNP no. | Bacterial species | ng/L | copies/350 μL |
| 653 | E. coli | 0.0029 | 5.29 × 10² |
| | E. cloacae | 0.0057 | 9.95 × 10² |
| | S. marcescens | 0.0063 | 1.14 × 10³ |
| | K. pneumoniae | 0.0007 | 1.22 × 10² |
| | P. mirabilis | 0.0030 | 6.85 × 10² |
| 412 | S. aureus | 0.0067 | 2.2 × 10³ |
| | S. epidermidis | 0.02 | 7.13 × 10³ |
| 378 | E. faecalis | 0.00267 | 7.68 × 10² |
| | E. faecium | 0.00311 | 1.07 × 10³ |
| | S. agalactiae | 0.00199 | 8.54 × 10² |
| | S. pyogenes | 0.00198 | 9.92 × 10² |
| 488 | S. pneumoniae | 0.0265 | 1.2 × 10⁴ |
| 408 | P. aeruginosa | 0.3942 | 5.83 × 10⁴ |
| 440 | H. influenzae | 0.0011 | 5.57 × 10² |
| 440 | B. fragilis | 0.0060 | 3.64 × 10² |

TABLE 14
Limit of Detection (LOD) values for the patient
samples of the fourteen bacterial species
assayed-Bacterial quantitation in patient samples.

| SNP | Bacterial | | Genome | Cells/ |
|---|---|---|---|---|
| no. | species | ng/μL | copies/350 μL | 350 μL |
| 653 | E. coli | 1.3 × 10⁻² | 2.37 × 10³ | 338 |
| | | 3.5 × 10⁻² | 6.38 × 10³ | 911 |
| | | 1.5 × 10⁻² | 2.74 × 10³ | 391 |
| | S. marcescens | 1.1 × 10⁻⁴ | 1.99 × 10¹ | 3 |
| | E. cloacae | 7 × 10⁻⁴ | 1.22 × 10² | 17 |
| | K. pneumoniae | 1.5 × 10⁻⁴ | 2.62 × 10¹ | 3 |
| | | 6.3 × 10⁻⁴ | 1.2 × 10² | 15 |
| | | 9 × 10⁻⁵ | 1.57 × 10¹ | 2 |
| 412 | S. aureus | 5.2 × 10⁻³ | 1.71 × 10³ | 342 |
| | | 7 × 10⁻³ | 2.3 × 10³ | 460 |
| | | 2.4 × 10⁻⁵ | 7.88 × 10⁰ | 2 |
| | | 3.5 × 10⁻⁴ | 9.86 × 10¹ | 20 |
| 408 | P. aeruginosa | 2.3 × 10⁻² | 3.4 × 10³ | 850 |
| | | 1.6 | 2.37 × 10⁵ | 59,250 |
| | | 8.3 × 10⁻² | 1.23 × 10⁴ | 3075 |

### Conclusion

This study demonstrates InfectID®'s ability to detect very low genome copies of the tope 20 bacterial species that are known to cause blood stream infections and sepsis. This was demonstrated in both spiked blood and patient blood samples.

### Example 3

380 patient whole blood samples obtained from patients that were admitted to the Royal Brisbane and Women's Hospital and the Mackay Base Hospital were investigated. Blood Culture (BacTAlert) results were obtained from Pathology Queensland. Furthermore, the same samples were subjected to InfectID®, which involved identifying the bacteria present in the sample, and using the methods of the present invention to quantify the bacterial cells present in 350 μL of blood (this was achieved simultaneously in the same assessment). Finally, all patient clinical metadata was evaluated and categorised to rank the patients as high, moderate and low risk based on clinical assessment criteria. The results are provided in Table 17 below.

### Blood Culture Process:

Blood cultures are used to check for the presence of a systemic bloodstream infection. Two or more blood samples were drawn from separate sites (commonly from veins in a patient's arms). The collection of multiple samples may increase the chance of detecting the infection. After collection, the blood is transferred into a blood culture bottle, such as the BacT/ALERT® culture media. These bottles are sent to a routine diagnostic laboratory where the bottles are placed into an incubation system, such as the BacT/ALERT® machine. If a bacterium or yeast is present in the patient blood, then these micro-organisms will grow in the blood culture bottle, and CO₂ is generated. The machine regularly measures the amount of CO₂, and once a threshold amount is produced, the blood culture bottle is flagged and the laboratory technician will remove the bottle from the machine and process the patient sample for identification of the micro-organism using standard microbiological culture techniques.

### InfectID® Process

In absolute quantification using the standard curve method, unknowns are quantitated based on a known quantity. Firstly, a standard curve is generated by using a tenfold serially diluted reference DNA of a known concentration, and this is used as the input sample for the InfectID® test. These reactions are run alongside the unknown samples and then the unknowns are compared to the standard curve and a value is extrapolated from the standard curve, which is based on the cycle time (CO from the real-time PCR amplification.

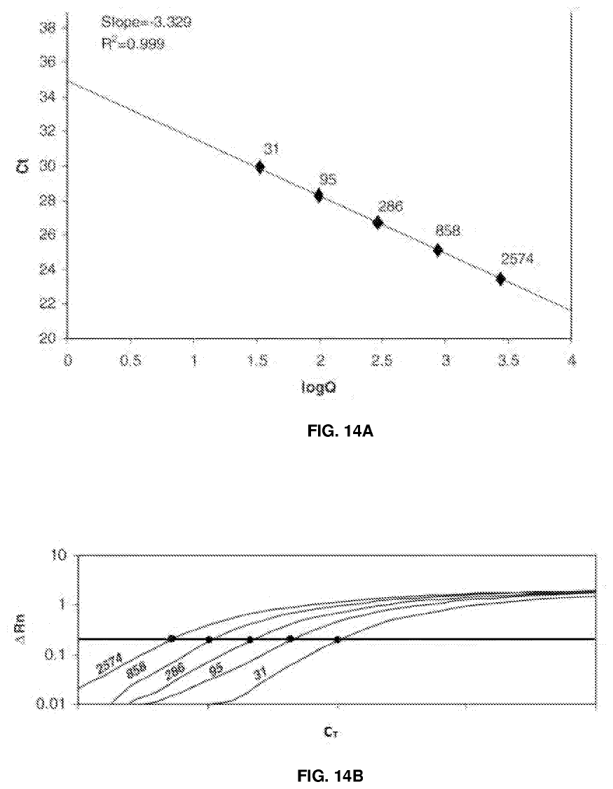

An example of a typical standard curve of C_{T} versus log copy number is illustrated in FIG. 14A (Yun J J, Heisler L, Hwang I I L, Wilkins O. 2006. Genomic DNA functions as a universal external standard in quantitative real-time PCR. Nucleic Acids Research, 34(12): e85). The points comprising the line are labelled with the copy number. FIG. 14B illustrates C_{T} values obtained from amplification plots which indicate the change in normalized signal for the five standards (indicated with copy numbers) between cycles 20 and 40 of the PCR. Cₜ is the cycle at which fluorescence crosses a threshold value.

The InfectID® Process was performed similarly to that of Example 2. In brief:
1. A reference sample (see Table 15) of a known concentration was diluted tenfold to provide a reference sample. A patient blood sample described above was also used.
2. DNA was extracted from the reference sample and the patient blood sample using the Roche MagNApure system.
3. Quantitative Real-Time PCR (qPCR) under the same conditions was performed on the reference sample and the patient blood sample. This determines if a bacterial species was present in the patient blood sample. If so, the qPCR data was analysed as outlined above to determine the concentration of bacteria present. The qPCR process was: One microliter of extracted DNA (1 to 3 ng) was added to 19 μl of reaction mastermix containing 10 μl of the 2× Type-it-HRM® Mastermix (Qiagen, Australia) and 8 pmol of each primer (primers were designed to amplify the regions encompassing the highly discriminatory SNPs in 16S rRNA (see SEQ ID Nos. 16-37 and 48-51)). Temperature cycling for these reactions were as follows: 50° C. for 2 min, 95° C. for 2 min, followed by 40 cycles of 95° C. for 15 s, 52° C. for 20 s, and 72° C. for 35 s, Hold at 72° C. for 2 min, Hold at 50° C. for 20 s (RotorGeneQ, Qiagen, Australia). PCR products sizes ranged from 79 bp to 96 bp.
4. The RotorGeneQ (Qiagen, Australia) software enables the user to visualize HRM data in multiple ways. The normalized raw melt curve depicts the decreasing fluorescence vs increasing temperature, and the difference curve, which displays a user-defined curve as the baseline (i.e. the x-axis), and depicts other normalized curves in relation to that baseline.
5. Using the formula described above, the concentration of the bacteria present was determined.

TABLE 15
Reference Sample data

| | | Geno | 16SrRN | | |
|---|---|---|---|---|---|
| | | me | A gene | GenBank | RefSeq |
| Bacterial | Strain | Size | copies/ | Assembly | Assembly |
| species | information | (Mb) | genome | Accession | Accession |
| Escherichia | ATCC11775 | 5.03 | 7 | GCA_003697165.2 | GCF_003697165.2 |
| coli | | | | | |
| Enterobacter | ATCC13047 | 5.6 | 8 | GCA_000025565.1 | GCF_000025565.1 |
| cloacae | | | | | |
| Proteus | ATCC29906 | 4.03 | 1 | GCA_000160755.1 | GCF_000160755.1 |
| mirabilis | | | | | |
| Morganella | ATCC25830 | 3.89 | 7 | GCA_006094455.1 | GCF_006094455.1 |
| morganii | | | | | |
| Serratia | ATCC274 | 5.15 | 7 | GCA_009936295.1 | GCF_009936295.1 |
| marcescens | | | | | |
| Acinetobacter | ATCC19606 | 4.0 | 6 | GCA_009035845.1 | GCF_009035845.1 |
| baumannii | | | | | |
| Shewanella | ATCC8071 | 4.39 | 8 | GCA_016406325.1 | GCF_016406325.1 |
| putrefaciens | | | | | |
| Citrobacter | ATCC8090 | 4.96 | 8 | GCA_011064845.1 | GCF_011064845.1 |
| freundii | | | | | |
| Klebsiella | ATCC BAA- | 5.78 | 8 | GCA_000364385.3 | GCF_000364385.3 |
| pneumoniae | 2146 | | | | |
| Staphylococcus | ATCC12600 | 2.78 | 6 | GCA_006094915.1 | GCF_006094915.1 |
| aureus | | | | | |
| Staphylococcus | ATCC14990 | 2.49 | 6 | GCA_006094375.1 | GCF_006094375.1 |
| epidermidis | | | | | |
| Aerococcus | ATCC11563 | 2.01 | 1 | GCA_000178435.1 | GCF_000178435.1 |
| viridans | | | | | |
| Streptococcus | ATCC49619 | 2.1 | 4 | GCA_003966485.1 | GCF_003966485.1 |
| pneumoniae | | | | | |
| Elaemophilus | NCTC8143 | 1.89 | 6 | GCA_001457655.1 | GCF_001457655.1 |
| influenzae | | | | | |
| Pseudomonas | ATCC27853 | 6.83 | 4 | GCA_001687285.1 | GCF_001687285.1 |
| aeruginosa | | | | | |
| Stenotrophomo | NCTC10258 | 4.48 | 4 | GCA_900475405.1 | GCF_900475405.1 |
| nasmaltophilia | | | | | |
| Enterococcus | ATCC29212 | 3.05 | 4 | GCA_000742975.1 | GCF_000742975.1 |
| faecalis | | | | | |
| Enterococcus | AUSMDU00004142 | 3.2 | 6 | GCA_003020765.1 | GCF_003020765.1 |
| faecium | | | | | |
| Streptococcus | NCTC13949 | 2.03 | 7 | GCA_900638415.1 | GCF_900638415.1 |
| agalactiae | | | | | |
| Streptococcus | NCTC8198 | 1.91 | 6 | GCA_002055535.1 | GCF_002055535.1 |
| pyogenes | | | | | |
| Streptococcus | NCTC10713 | 1.95 | 4 | GCA_900636475.1 | GCF_900636475.1 |
| anginosus | | | | | |
| Enterococcus | ATCC43198 | 2.41 | 6 | GCA_000407565.1 | GCF_000407565.1 |
| cecorum | | | | | |
| Cedecea | NCTC11466 | 4.78 | 7 | GCA_900635955.1 | GCF_900635955.1 |
| lapagei | | | | | |
| Cronobacter | LMG23823 | 4.63 | 7 | GCA_001277235.1 | GCF_001277235.1 |
| dublinensis | | | | | |

### Clinical Assessment Criteria

Patient metadata was evaluated and categorised according to the Emergency Department Adult Sepsis Pathway for Tertiary and Secondary Facilities, Queensland, in order to rank the patients as high, moderate and low based on the clinical assessment criteria (provided in Table 16).

TABLE 16
Risk Factor Categories: Emergency Department Adult
Sepsis Pathway for Tertiary
and Secondary Facilities Queensland

| Risk Category | Risk Criteria |
|---|---|
| High | Systolic Blood Pressure <90 mmHg |
| | (or drop >40 from normal) |
| | Lactate ≥2 mmol/L if known |
| | Non-blanching rash/Mottled/Ashen/Cyanotic |
| | Respiratory rate ≥25 breaths/min |
| | Needs oxygen to keep oxygen saturation ≥92% |
| | Heart rate ≥130 beats/min |
| | Change in mental status (Glasgow coma scale <15) |
| | Not passes urine in last 18 hours OR urinary |
| | output (UO) <0.5 m L/kg/hr (if known) |
| | Recent chemotherapy |
| Moderate | Respiratory rate 21-24 breaths/min |
| | Systolic blood pressure 90-99 mmHg |
| | Heart rate 90-129 beats/min OR new dysrhythmia |
| | Temperature <35.5° C. of ≥38.5° C. |
| | Relatives concerned about mental health status |
| | Not passes urine in last 12-18 hours |
| | Acute deterioration in functional ability |
| Low | Look for other common causes of deterioration |
| | In the event of deterioration, reassess the patient |

TABLE 17
Bacterial cell quantification of patient blood samples using the InfectID ® test

| | | | | Emergency Department Adult | | |
|---|---|---|---|---|---|---|
| | | | Number | Sepsis Pathway for Tertiary and | | |
| | | | of | Secondary Facilities | | |
| | | | bacterial | Presence of Patient Risk Factors | | |
| | | | cells in | (1 = present; 0 = absent) | | |
| Patient | | BacTAlert ® | 350 μL | High | Moderate | Low |
| sample | InfectID ® | blood culture | patient | risk | risk | risk |
| number | result | result | blood | criteria | criteria | criteria |
| 1 | Escherichia | Escherichia | 1237 | 1 | 0 | 0 |
| | coli | coli | | | | |
| 2 | Escherichia | Escherichia | 17,499 | 1 | 1 | 0 |
| | coli | coli | | | | |
| 3 | Escherichia | Escherichia | 4968 | 1 | 0 | 0 |
| | coli | coli | | | | |
| 4 | Escherichia | Escherichia | 289 | 0 | 1 | 0 |
| | coli | coli | | | | |
| 5 | Enterobacter | Enterobacter | 56 | 1 | 1 | 0 |
| | cloacae | cloacae | | | | |
| 6 | Enterobacter | No growth | 6424 | 1 | 1 | 0 |
| | cloacae | | | | | |
| 7 | Enterobacter | No growth | 1247 | 0 | 0 | 1 |
| | cloacae | | | | | |
| 8 | Enterobacter | Enterobacter | 56 | 1 | 1 | 0 |
| | cloacae | cloacae | | | | |
| 9 | Enterobacter | No growth | 317 | 0 | 1 | 0 |
| | cloacae | | | | | |
| 10 | Shewanella | No growth | 76 | 0 | 0 | 1 |
| | putrefaciens | | | | | |
| 11 | Shewanella | No growth | 546 | 0 | 0 | 1 |
| | putrefaciens | | | | | |
| 12 | Serratia | No growth | 53 | 0 | 1 | 0 |
| | marcescens | | | | | |
| 13 | Serratia | Enterobacter | 1183 | 1 | 1 | 0 |
| | marcescens | cloacae | | | | |
| 14 | Serratia | No growth | 77 | 0 | 0 | 1 |
| | marcescens | | | | | |
| 15 | Citrobacter | Klebsiella | 216 | 0 | 0 | 1 |
| | freundii | oxytoca | | | | |
| 16 | Staphylococcus | No growth | 3258 | 1 | 1 | 0 |
| | aureus | | | | | |
| 17 | Staphylococcus | Staphylococcus | 1 | 1 | 1 | 0 |
| | aureus | epidermidis | | | | |
| 18 | Staphylococcus | No growth | 4 | 0 | 0 | 1 |
| | aureus | | | | | |
| 19 | Staphylococcus | Staphylococcus | 188 | 0 | 1 | 0 |
| | aureus | aureus | | | | |
| | | (MRSA) | | | | |
| 20 | Staphylococcus | No growth | 45 | 0 | 0 | 1 |
| | aureus | | | | | |
| 21 | Staphylococcus | No growth | 239 | 0 | 1 | 0 |
| | epidermidis | | | | | |
| 22 | Staphylococcus | No growth | 14 | 1 | 1 | 0 |
| | epidermidis | | | | | |
| 23 | Staphylococcus | No growth | 1 | 1 | 1 | 0 |
| | epidermidis | | | | | |
| 24 | Aerococcus | No growth | 318 | 1 | 0 | 0 |
| | viridans | | | | | |
| 25 | Aerococcus | No growth | 11 | 0 | 1 | 0 |
| | viridans | | | | | |
| 26 | Aerococcus | No growth | 7 | 1 | 1 | 0 |
| | viridans | | | | | |
| 27 | Aerococcus | No growth | 1 | 1 | 1 | 0 |
| | viridans | | | | | |
| 28 | Streptococcus | No growth | 410 | 0 | 1 | 0 |
| | pneumoniae | | | | | |
| 29 | Streptococcus | Escherichia | 201 | 0 | 0 | 1 |
| | pneumoniae | coli | | | | |
| 30 | Streptococcus | Escherichia | 1 | 0 | 1 | 0 |
| | pneumoniae | coli | | | | |
| 31 | Streptococcus | Pseudomonas | 1 | 0 | 1 | 0 |
| | pneumoniae | aeruginosa | | | | |
| 32 | Enterococcus | Escherichia | 71 | 0 | 0 | 1 |
| | cecorum | coli | | | | |
| 33 | Cedecea | No growth | 67 | 1 | 1 | 0 |
| | lapagei | | | | | |
| 34 | Cronobacter | No growth | 47 | 1 | 1 | 0 |
| | dublinensis | | | | | |
| 35 | Cronobacter | Enterobacter | 59 | 1 | 1 | 0 |
| | dublinensis | cloacae | | | | |

### REFERENCES

1. Reimer L G, Wilson M L, Weinstein M P. Update on detection of bacteremia and fungemia. Clin Microbiol Rev. 1997; 10:444-65.
2. Yagupsky P., Nolte F. 1990. Quantitative aspects of septicemia. Clin. Microbiol. Rev. 3:269-279.
3. Reier-Nilsen T., Farstad T., Nakstad B., Lauvrak V., Steinbakk M. 2009. Comparison of broad range 16S rDNA PCR and conventional blood culture for diagnosis of sepsis in the newborn: a case control study. BMC Pediatr. 9:5.
4. Smith, J., Y. Serebrennikova, D. Huffman, G. Leparc, and L. Garcia Rubio. 2008. A new method for the detection of microorganisms in blood cultures: Part I. Theoretical analysis and simulation of blood culture processes. Can. J. Chem. Eng. 86:947-959.
5. Hall, W. H., and D. Gold. 1955. Shock associated with bacteremia. Arch. Intern. Med. 96:403-412.
6. Weil, M. H., and W. W. Spink. 1958. The shock syndrome associated with bacteremia due to gram-negative bacilli. Arch. Intern. Med. 101:184-193.
7. Yun J J, Heisler L, Hwang I I L, Wilkins O. 2006. Genomic DNA functions as a universal external standard in quantitative real-time PCR. Nucleic Acids Research, 34(12): e85.

1-21. (canceled)

22. A method of determining the quantity or concentration of a bacterium in a sample, said method including the steps of:
(a) amplifying a target nucleic acid of the bacterium from genetic material obtained from the sample to form an amplification product, wherein the target nucleic acid comprises at least a portion of a bacterial 16S rRNA gene and wherein the target nucleic acid comprises one or a plurality of single nucleotide polymorphisms (SNPs) in the bacterial 16S rRNA gene;
(b) identifying the bacterium in the sample and measuring a quantity or concentration of the amplification product in a single process, wherein the step of identifying the bacterium comprises analysing the amplification product for the presence or absence of one or a plurality of SNPs;
(c) calculating a quantity or concentration of the target nucleic acid in the sample by comparing the quantity or concentration of the amplification product with a reference level thereof; and
(d) determining a copy number of the bacterial 16S rRNA gene in the sample from the quantity or concentration of the target nucleic acid therein, the copy number being a function of or correlated to the quantity of the bacterium in the sample.

23. The method of claim 22, wherein the one or plurality of SNPs corresponding to at least one of positions 273, 378, 408, 412, 440, 488, 647, 653, 737, 755, 762 and 776 of the 16S rRNA gene set forth in SEQ ID NO: 1.

24. The method of claim 22, further including the step of generating the reference level from one or a plurality of control samples.

25. The method of claim 24, wherein the step of generating the reference level comprises amplifying the target nucleic acid from the one or plurality of control samples, wherein the control samples comprise genetic material of a known quantity or concentration.

26. The method of claim 24, wherein amplifying the target nucleic acid from the one or plurality of control samples is performed substantially simultaneously or in parallel with step (a).

27. The method of claim 24, wherein the one or plurality of control samples further comprise genetic material from one or a plurality of further bacteria.

28. The method of claim 22, wherein amplifying the target nucleic acid from genetic material of the sample and/or the one or plurality of control samples is conducted with a pair of primers that comprise at least one of SEQ ID NOs: 16-37 and 48-51.

29. The method of claim 22, wherein the step of analysing the amplification product for the presence or the absence of the at least one SNP comprises the use of high resolution melt analysis, 5′ nuclease digestion, molecular beacons, oligonucleotide ligation, microarray, restriction fragment length polymorphism, antibody detection methods, direct sequencing or any combination thereof.

30. The method of claim 22, wherein the copy number of the bacterial 16S rRNA gene is determined using the formula:
$$copy number = \frac{X ng * 6.0221 \times {10}^{23} molecules / mole}{( N * 660 g / mole ) * 1 \times {10}^{9} ng / g}$$
wherein X is the quantity of the amplification product and N is the length in nucleotides of the target nucleic acid.

31. The method of claim 22, wherein the sample is a biological sample, such as sputum, blood, cerebrospinal fluid or urine, taken from a subject.

32. The method of claim 31, wherein the subject has an infection by the bacterium.

33. A method of determining a prognosis for an infection by a bacterium in a subject, including the step of quantifying the bacterium in a biological sample from the subject according to the method of claim 22 to thereby evaluate the prognosis of the infection in the subject.

34. A method of treating an infection by a bacterium in a subject including the steps of:
quantifying the bacterium in a biological sample from the subject according to the method of claim 22, and based on the quantification made, initiating, continuing, modifying or discontinuing a treatment of the infection.

35. A method of evaluating treatment efficacy of an infection by a bacterium in a subject including:
quantifying the bacterium in a biological sample from the subject according to the method of claim 22; and
determining whether or not the treatment is efficacious according to whether said quantity of the bacterium in the subject's biological sample is reduced or absent.

36. The method of claim 33, wherein the subject has sepsis.

37. A kit or assay for quantifying a bacterium in a sample or biological sample, said kit or assay comprising one or more reagents for performing the method according to claim 22 and instructions for use.


SEQUENCE LISTING (51 sequences)

SEQ ID NO: 1 - DNA, 1542 bp, Escherichia coli
aaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacagaactttccagagatggattggtgccttcgggaactgtgagacaggtgctgcatggctgtcgtcagctcgtgttgtgaaatgttgggttaagtcccgcaacgagcgcaacccttatcttttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 2 - DNA, 1555 bp, Staphylococcus aureus
ttttatggagagtttgatcctggctcaggatgaacgctggcggcgtgcctaatacatgcaagtcgagcgaacggacgagaagcttgcttctctgatgttagcggcggacgggtgagtaacacgtggataacctacctataagactgggataacttcgggaaaccggagctaataccggataatattttgaaccgcatggttcaaaagtgaaagacggtcttgctgtcacttatagatggatccgcgctgcattagctagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagctttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgtgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttaggagctagccgtcgaaggtgggacaaatgattggggtgaagtcgtaacaaggtagccgtatcggaaggtgcggctggatcacctcctttct

SEQ ID NO: 3 - DNA, 1554 bp, Staphylococcus epidermidis
ttttatggagagtttgatcctggctcaggatgaacgctggcggcgtgcctaatacatgcaagtcgagcgaacagacgaggagcttgcttctctgacgttagcggcggacgggtgagtaacacgtggataacctacctataagactgggataacttcgggaaaccggagctaataccggataatatattgaaccgcatggttcaatagtgaaagacggttttgctgtcacttatagatggatccgcgccgcattagctagttggtaaggtaacggcttaccaaggcaacgatgcgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaatgtgtaagtaactatgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctctgacccctctagagatagagttttccccttcgggggacagagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactatatgaagctggaatcgctagtaatcgtagatcagcatgctacggtgaatacgttcccgggtcttgtacacaccgcccgtcacaccacgagagtttgtaacacccgaagccggtggagtaaccatttggagctagccgtcgaaggtgggacaaatgattggggtgaagtcgtaacaaggtagccgtatcggaaggtgcggctggatcacctcctttct

SEQ ID NO: 4 - DNA, 1514 bp, Streptococcus pneumoniae
gtttgatcctggctcaggacgaacgctggcggcgtgcctaatacatgcaagtagaacgctgaaggaggagcttgcttctctggatgagttgcgaacgggtgagtaacgcgtaggtaacctgcctggtagcgggggataactattggaaacgatagctaataccgcataagagtggatgttgcatgacatttgcttaaaaggtgcacttgcatcactaccagatggacctgcgttgtattagctagttggtggggtaacggctcaccaaggcgacgatacatagccgacctgagagggtgatcggccacactgggactgagacacggcccagactcctacgggaggcagcagtagggaatcttcggcaatggacggaagtctgaccgagcaacgccgcgtgagtgaagaaggttttcggatcgtaaagctctgttgtaagagaagaacgagtgtgagagtggaaagttcacactgtgacggtatcttaccagaaagggacggctaactacgtgccagcagccgcggtaatacgtaggtcccgagcgttgtccggatttattgggcgtaaagcgagcgcaggcggttagataagtctgaagttaaaggctgtggcttaaccatagtaggctttggaaactgtttaacttgagtgcaagaggggagagtggaattccatgtgtagcggtgaaatgcgtagatatatggaggaacaccggtggcgaaagcggctctctggcttgtaactgacgctgaggctcgaaagcgtggggagcaaacaggattagataccctggtagtccacgctgtaaacgatgagtgctaggtgttagaccctttccggggtttagtgccgtagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccctctgaccgctctagagatagagttttccttcgggacagaggtgacaggtggtgcatggttgtcgtcagctcgtgtcgtgagatgttgggttaagtcccgcaacgagcgcaacccctattgttagttgccatcattcagttgggcactctagcgagactgccggtaataaaccggaggaaggtggggatgacgtcaaatcatcatgccccttatgacctgggctacacacgtgctacaatggctggtacaacgagtcgcaagccggtgacggcaagctaatctcttaaagccagtctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccgtaaggagccagccgcctaaggtgggatagatgattggggtgaagtcgtaacaaggtagccgtatcggaag

SEQ ID NO: 5 - DNA, 1501 bp, Streptococcus agalactiae
gacgaacgctggcggcgtgcctaatacatgcaagtagaacgctgaggtttggtgtttacactagactgatgagttgcgaacgggtgagtaacgcgtaggtaacctgcctcatagcgggggataactattggaaacgatagctaataccgcataagagtaattaacacatgttagttatttaaaaggagcaattgcttcactgtgagatggacctgcgttgtattagctagttggtgaggtaaaggctcaccaaggcgacgatacatagccgacctgagagggtgatcggccacactgggactgagacacggcccagactcctacgggaggcagcagtagggaatcttcggcaatggacggaagtctgaccgagcaacgccgcgtgagtgaagaaggttttcggatcgtaaagctctgttgttagagaagaacgttggtaggagtggaaaatctaccaagtgacggtaactaaccagaaagggacggctaactacgtgccagcagccgcggtaatacgtaggtcccgagcgttgtccggatttattgggcgtaaagcgagcgcaggcggttctttaagtctgaagttaaaggcagtggcttaaccattgtacgctttggaaactggaggacttgagtgcagaaggggagagtggaattccatgtgtagcggtgaaatgcgtagatatatggaggaacaccggtggcgaaagcggctctctggtctgtaactgacgctgaggctcgaaagcgtggggagcaaacaggattagataccctggtagtccacgccgtaaacgatgagtgctaggtgttaggccctttccggggcttagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccttctgaccggcctagagataggctttctcttcggagcagaagtgacaggtggtgcatggttgtcgtcagctcgtgtcgtgagatgttgggttaagtcccgcaacgagcgcaacccctattgttagttgccatcattaagttgggcactctagcgagactgccggtaataaaccggaggaaggtggggatgacgtcaaatcatcatgccccttatgacctgggctacacacgtgctacaatggttggtacaacgagtcgcaagccggtgacggcaagctaatctcttaaagccaatctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccttttaggagccagccgcctaaggtgggatagatgattggggtgaagtcgtaacaaggtagccgtatcggaagg

SEQ ID NO: 6 - DNA, 1543 bp, Streptococcus pyogenes
gagagtttgatcctggctcaggacgaacgctggcggcgtgcctaatacatgcaagtagaacgctgagaactggtgcttgcaccggttcaaggagttgcgaacgggtgagtaacgcgtaggtaacctacctcatagcgggggataactattggaaacgatagctaataccgcataagagagactaacgcatgttagtaatttaaaaggggcaattgctccactatgagatggacctgcgttgtattagctagttggtgaggtaaaggctcaccaaggcgacgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaagctctgttgttagagaagaatgatggtgggagtggaaaatccaccaagtgacggtaactaaccagaaagggacggctaactacgtgccagcagccgcggtaatacgtaggtcccgagcgttgtccggatttattgggcgtaaagcgagcgcaggcggttttttaagtctgaagttaaaggcattggctcaaccaatgtacgctttggaaactggagaacttgagtgcagaaggggagagtggaattccatgtgtagcggtgaaatgcgtagatatatggaggaacaccggtggcgaaagcggctctctggtctgtaactgacgctgaggctcgaaagcgtggggagcaaacaggattagataccctggtagtccacgccgtaaacgatgagtgctaggtgttaggccctttccggggcttagtgccggagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcccgatgcccgctctagagatagagttttacttcggtacatcggtgacaggtggtgcatggttgtcgtcagctcgtgtcgtgagatgttgggttaagtcccgcaacgagcgcaacccctattgttagttgccatcattaagttgggcactctagcgagactgccggtaataaaccggaggaaggtggggatgacgtcaaatcatcatgccccttatgacctgggctacacacgtgctacaatggttggtacaacgagtcgcaagccggtgacggcaagctaatctcttaaagccaatctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaacctattaggagccagccgcctaaggtgggatagatgattggggtgaagtcgtaacaaggtagccgtatcggaaggtgcggctggatcacctccttt

SEQ ID NO: 7 - DNA, 1522 bp, Enterococcus faecalis
agagtttgatcctggctcaggacgaacgctggcggcgtgcctaatacatgcaagtcgaacgcttctttcctcccgagtgcttgcactcaattggaaagaggagtggcggacgggtgagtaacacgtgggtaacctacccatcagagggggataacacttggaaacaggtgctaataccgcataacagtttatgccgcatggcataagagtgaaaggcgctttcgggtgtcgttgatggatggacccgcggtgcattagctagttggtgaggtaacggctcaccaaggccacgatgcatagccgacctgagagggtgatcggccacactgggactgagacacggcccagactcctacgggaggcagcagtagggaatcttcggcaatggacgaaagtctgaccgagcaacgccgcgtgagtgaagaaggttttcggatcgtaaaactctgttgttagagaagaacaaggacgttagtaactgaacgtcccctgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagcaaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagctttcccttcggggacaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgctagaccgcgaggtcatgcaaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagtcggtgaggtaacctttttggagccagccgcctaaggtgggatagatgattggggtgaagtcgtaacaaggtagcc

SEQ ID NO: 8 - DNA, 1551 bp, Enterococcus faecium
agagtttgatcctggctcaggacgaacgctggcggcgtgcctatacatgcaagtcgaacgcttctttttccaccggagcttgctccaccggaaaaagaggagtggcgaacgggtgagtaacacgtgggtaacctgcccatcagaaagggataacacttggaaacaggtgctaataccgtataacaaatcaaaaccgcatggttttgatttgaaaggcgctttcgggtgtcgctgatggatggacccgcggtgcattagctagttggtgaggtaacggctcaccaaggccacgatgcatagccgcacctgagagggtgatcggccacattgggactgagacacggcccaaactctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacacgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgcaggctgcaactcgcctgcatgaagccggaatcgctagtaatcgcggatcagcacgccgcgtgaatacgttcccgggccttgtacacaccgcccgtcacaccacgagagtttgtaacacccgaagtcggtgaggtaaccttttggagccagccgcctaaggtgggatagatgattggggtgaagtcgtaacaaggtagccgtatctgaaggtgcggctggatcacctccttt

SEQ ID NO: 9 - DNA, 1542 bp, Proteus mirabilis
aattgaagagtttgatcatggctcagattgaacgctggcggcaggcctaacacatgcaagtcgagcggtaacaggagaaagcttgctttcttgctgacgagcggcggacgggtgagtaatgtatggggatctgcccgatagagggggataactactggaaacggtggctaataccgcataatgtctacggaccaaagcaggggctcttcggaccttgcactatcggatgaacccatatgggattagctagtaggtggggtaaaggctcacctaggcgacgatctctagctggtctgagaggatgatcagccacactgggactgagacacggcccagactcctacgggaggcagcagtggggaatattgcacaatgggcgcaagcctgatgcagccatgccgcgtgtatgaagaaggccttagggttgtaaagtactttcagcggggaggaaggtgataaggttaatacccttgtcaattgacgttacccgcagaagaagcaccggctaactccgtgccagcagccgcggtaatacggagggtgcaagcgttaatcggaattactgggcgtaaagcgcacgcaggcggtcaattaagtcagatgtgaaagccccgagcttaacttgggaattgcatctgaaactggttggctagagtcttgtagaggggggtagaattccatgtgtagcggtgaaatgcgtagagatgtggaggaataccggtggcgaaggcggccccctggacaaagactgacgctcaggtgcgaaagcgtggggagcaaacaggattagataccctggtagtccacgctgtaaacgatgtcgatttagaggttgtggtcttgaaccgtggcttctggagctaacgcgttaaatcgaccgcctggggagtacggccgcaaggttaaaactcaaatgaattgacgggggcccgcacaagcggtggagcatgtggtttaattcgatgcaacgcgaagaaccttacctactcttgacatccagcgaatcctttagagatagaggagtgccttcgggaacgctgagacaggtgctgcatggctgtcgtcagctcgtgttgtgaaatgttgggttaagtcccgcaacgagcgcaacccttatcctttgttgccagcacgtaatggtgggaactcaaaggagactgccggtgataaaccggaggaaggtggggatgacgtcaagtcatcatggcccttacgagtagggctacacacgtgctacaatggcagatacaaagagaagcgacctcgcgagagcaagcggaactcataaagtctgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 10 - DNA, 1505 bp, Serratia marcescens
gctcagattgaacgctggcggcaggcttaacacatgcaagtcgagcggtagcacaggggagcttgctccctgggtgacgagcggcggacgggtgagtaatgtctgggaaactgcctgatggagggggataactactggaaacggtagctaataccgcataacgtcgcaagaccaaagagggggaccttcgggcctcttgccatcagatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcgaggaggaaggtggtgagcttaatacgttcatcaattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatttgaaactggcaagctagagtctcgtagaggggggtagaattccaggtgtagcggtgaaatgcgtagagatctggaggaataccggtggcgaaggcgggcccctggacgaagactgacgctcaggtgccaaagcgtggggagcaaacaggattagataccctggtagtccacgctgtaaacgatgtcgatttggaggttgtgcccttgaggcgtggcttccggagctaacgcgttaaatcgaccgcctggggagtacggccgcaaggttaaaactcaaatgaattgacgggggcccgcacaagcggtggagcatgtggtttaattcgatgcaacgcgaagaaccttacctactcttgacatccagagaactttccagagatggattggtgccttcgggaactctgagacaggtgctgcatggctgtcgtcagctcgtgttgtgaaatgttgggttaagtcccgcaacgagcgcaacccttatcctttgttgccagcggttcggccgggaactcaaaggagactgccagtgataaactggaggaaggtggggatgacgtcaagtcatcatggcccttacgagtagggctacacacgtgctacaatggcatatacaaagagaagcgacctcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcgg

SEQ ID NO: 11 - DNA, 1438 bp, Enterobacter aerogenes
  misc_feature (1425)..(1425): n is a, c, g, or t
acgctggcggcaggcctaacacatgcaagtcgagcggtagcacagagagcttgctctcgggtgacgagcggcggacgggtgagtaatgtctgggaaactgcctgatggagggggataactactggaaacggtagctaataccgcataacgtcgcaagaccaaagtgggggaccttcgggcctcatgccatcagatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcgaggaggaaggcgttaaggttaataaccttggcgattgacgttactcgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaacttagcagagatgctttggtgccttcgggaactctgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgagtagggctacacacgtgctacaatggcatatacaaagagaagcgacctcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggaggncgctttaccactt

SEQ ID NO: 12 - DNA, 1511 bp, Enterobacter cloacae
tgaacgctggcggcaggcctaacacatgcaagtcgaacggtagcacagagagcttgctctcgggtgacgagtggcggacgggtgagtaatgtctgggaaactgcctgatggagggggataactactggaaacggtagctaataccgcataaygtcgcaagaccaaagagggggaccttcgggcctcttgccatcagatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggtgttgtggttaataaccgcagcaattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctggagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgatttggaggttgtgcccttgaggcgtggcttccggagctaacgcgttaaatcgaccgcctggggagtacggccgcaaggttaaaactcaaatgaattgacgggggcccgcacaagcggtggagcatgtggtttaattcgatgcaacgcgaagaaccttacctggtcttgacatccacagaactttccagagatggattggtgccttcgggaactgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggctggatcacctccttg

SEQ ID NO: 13 - DNA, 1534 bp, Klebsiella pneumoniae
  misc_feature (11)..(12): n is a, c, g, or t
agagtttgatnntggctcagattgaacgctggcggcaggcctaacacatgcaagtcgagcggtagcacagagagcttgctctcgggtgacgagcggcggacgggtgagtaatgtctgggaaactgcctgatggagggggataactactggaaacggtagctaataccgcataacgtcgcaagaccaaagtgggggaccttcgggcctcatgccatcagatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcggggaggaaggcgatgaggttaataacctcatcgattgacgttaccctgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgatttggaggttgtgcccttgaggcgtggcttccggagctaacgcgttaaatcgaccgcctggggagtacggccgcaaggttaaaactcaaatgaattgacgggggcccgcacaagcggtggagcatgtggtttaattcgatgcaacgcgaagaaccttacctggtcttgacatccacagaactttccagagatggattggtgccttcgggaactgtgagacaggtgctgcatggctgtcgtcagctcgtgttgtgaaatgttgggttaagtcccgcaacgagcgcaacccttatcctttgttgccagcggttaggccgggaactcaaaggagactgccagtgataaactggaggaaggtggggatgacgtcaagtcatcatggcccttacgaccagggctacacacgtgctacaatggcatatacaaagagaagcgacctcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctccttt

SEQ ID NO: 14 - DNA, 1536 bp, Pseudomonas aeruginosa
gaactgaagagtttgatcatggctcagattgaacgctggcggcaggcctaacacatgcaagtcgagcggatgaagggagcttgctcctggattcagcggcggacgggtgagtaatgcctaggaatctgcctggtagtgggggataacgtccggaaacgggcgctaataccgcatacgtcctgagggagaaagtgggggatcttcggacctcacgctatcagatgagcctaggtcggattagctagttggtggggtaaaggcctaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagttaataccttgctgttttgacgttaccaacagaataagcaccggctaacttcgtgccagcagccgcggtaatacgaagggtgcaagcgttaatcggaattactgggcgtaaagcgcgcgtaggtggttcagcaagttggatgtgaaatccccgggctcaacctgggaactgcatccaaaactactgagctagagtacggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcgactagccgttgggatccttgagatcttagtggcgcagctaacgcgataagtcgaccgcctggggagtacggccgcaaggttaaaactcaaatgaattgacgggggcccgcacaagcggtggagcatgtggtttaattcgaagcaacgcgaagaaccttacctggccttgacatgctgagaactttccagagatggattggtgccttcgggaactcagacacaggtgctgcatggctgtcgtcagctcgtgtcgtgagatgttgggttaagtcccgtaacgagcgcaacccttgtccttagttaccagcacctcgggtgggcactctaaggagactgccggtgacaaaccggaggaaggtggggatgacgtcaagtcatcatggcccttacggccagggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgctccagaagtagctagtctaaccgcaagggggacggttaccacggagtgattcatgactggggtgaagtcgtaacaaggtagccgtaggggaacctgcggctggatcacctcctta

SEQ ID NO: 15 - DNA, 1497 bp, Acinetobacter calcoaceticus
tagagtttgatcctggctcagattgaacgctggcggcaggcttaacacatgcaagtcgagcggggaaaggtagcttgctactggacctagcggcggacgggtgagtaatgcttaggaatctgcctattagtgggggacaacattccgaaaggaatgctaataccgcatacgtcctacgggagaaagcaggggaccttcgggccttgcgctaatagatgagcctaagtcggattagctagttggtggggtaaaggcctaccaaggcgacgatctgtagcggtctgagaggatgatccgccacactgggactgagacacggcccagactcctacgggaggcagcagtggggaatattggacaatggggggaaccctgatccagccatgccgcgtgtgtgaagaaggccttatggttgtaaagcactttaagcgaggaggaggctactagtattaatactactggatagtggacgttactcgcagaataagcaccggctaactctgtgccagcagccgcggtaatacagagggtgcgagcgttaatcggatttactgggcgtaaagcgtgcgtaggcggccatttaagtcaaatgtgaaatccccgagcttaacttgggaattgcattgcatactggatggctagagtatgggagaggatggtagaattccaggtgtagcggtgaaatgcgtagagatctggaggaataccgatggcgaaggcagccatctggcctaatactgacgctgaggtacgaaagcatgggagcagaacaggattagataccctggtagtccatgccgtaaacgatgtttactagccgttggggcctttgaggctttagtggcgcagctaacgcgataagtagaccgcctggggagtacggtcgcaagactaaaactcaaatgaattgacgggggcccgcacaagcggtggagcatgtggtttaattcgatgcaacgcgaagaaccttacctggccttgacatactagaaactttccagagatggattggtgccttcgggaatttagatacaggtgctgcatggctgtcgtcagctcgtgtcgtgagatgttgggttaagtcccgcaacgagcgcaacccttttccttacttgccagcatttcggatgggaactttaaggatactgccagtgacaaactggaggaaggcggggacgacgtcaagtcatcatggcccttacggccagggctacacacgtgctacaatggtcggtacaaagggttgctacctagcgataggatgctaatctcaaaaagccgatcgtagtccggattggagtctgcaactcgactccatgaagtcggaatcgctagtaatcgcggatcagaatgcccggtgatacgttcccgggccttgtacacaccgcccgtcacaccatgggagtttgttgcaccagaagtaggtagtctaaccgcaaggaggacgcttaccacggtgtggccgatgactggggtgaagtcgtaacaaggtaacca

SEQ ID NO: 16 - DNA, 19 bp, Artificial Sequence
  Forward Primer
cctcttgccatcggatgtg

SEQ ID NO: 17 - DNA, 20 bp, Artificial Sequence
  Reverse Primer
ccagtgtggctggtcatcct

SEQ ID NO: 18 - DNA, 20 bp, Artificial Sequence
  Forward Primer
cctacgggaggcagcagtag

SEQ ID NO: 19 - DNA, 20 bp, Artificial Sequence
  Forward Primer
gggaggcagcagtagggaat

SEQ ID NO: 20 - DNA, 22 bp, Artificial Sequence
  Reverse Primer
cgatccgaaaaccttcttcact

SEQ ID NO: 21 - DNA, 26 bp, Artificial Sequence
  Forward Primer
aagacggtcttgctgtcacttataga

SEQ ID NO: 22 - DNA, 22 bp, Artificial Sequence
  Reverse Primer
ctatgcatcgttgccttggtaa

SEQ ID NO: 23 - DNA, 18 bp, Artificial Sequence
  Forward Primer
tgccgcgtgaatgaagaa

SEQ ID NO: 24 - DNA, 21 bp, Artificial Sequence
  Forward Primer
gcgtgaaggatgaaggctcta

SEQ ID NO: 25 - DNA, 21 bp, Artificial Sequence
  Forward Primer
tgatgaaggttttcggatcgt

SEQ ID NO: 26 - DNA, 29 bp, Artificial Sequence
  Reverse Primer
tgatgtactattaacacatcaaccttcct

SEQ ID NO: 27 - DNA, 22 bp, Artificial Sequence
  Reverse Primer
aacgctcggatcttccgtatta

SEQ ID NO: 28 - DNA, 21 bp, Artificial Sequence
  Reverse Primer
cgctcgccacctacgtattac

SEQ ID NO: 29 - DNA, 28 bp, Artificial Sequence
  Forward Primer
gttgtaagagaagaacgagtgtgagagt

SEQ ID NO: 30 - DNA, 22 bp, Artificial Sequence
  Reverse Primer
cgtagttagccgtccctttctg

SEQ ID NO: 31 - DNA, 24 bp, Artificial Sequence
  Forward Primer
gcggtttgttaagtcagatgtgaa

SEQ ID NO: 32 - DNA, 22 bp, Artificial Sequence
  Forward Primer
ggtctgtcaagtcggatgtgaa

SEQ ID NO: 33 - DNA, 21 bp, Artificial Sequence
  Forward Primer
tcaacctgggaactcattcga

SEQ ID NO: 34 - DNA, 22 bp, Artificial Sequence
  Reverse Primer
ggaattctacccccctctacga

SEQ ID NO: 35 - DNA, 23 bp, Artificial Sequence
  Reverse Primer
ggaattctacccccctctacaag

SEQ ID NO: 36 - DNA, 23 bp, Artificial Sequence
  Forward Primer
gtgtagcggtgaaatgcgtagag

SEQ ID NO: 37 - DNA, 23 bp, Artificial Sequence
  Reverse Primer
tcgtttaccgtggactaccaggg

SEQ ID NO: 38 - DNA, 1554 bp, Bacillus anthracis
ttattggagagtttgatcctggctcaggatgaacgctggcggcgtgcctaatacatgcaagtcgagcgaatggattaagagcttgctcttatgaagttagcggcggacgggtgagtaacacgtgggtaacctgcccataagactgggataactccgggaaaccggggctaataccggataacattttgaaccgcatggttcgaaattgaaaggcggcttcggctgtcacttatggatggacccgcgtcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtgctagttgaataagctggcaccttgacggtacctaaccagaaagccacggctaactacgtgccagcagccgcggtaatacgtaggtggcaagcgttatccggaattattgggcgtaaagcgcgcgcaggtggtttcttaagtctgatgtgaaagcccacggctcaaccgtggagggtcattggaaactgggagacttgagtgcagaagaggaaagtggaattccatgtgtagcggtgaaatgcgtagagatatggaggaacaccagtggcgaaggcgactttctggtctgtaactgacactgaggcgcgaaagcgtggggagcaaacaggattagataccctggtagtccacgccgtaaacgatgagtgctaagtgttagagggtttccgccctttagtgctgaagttaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgacaaccctagagatagggcttctccttcgggagcagagtgacaggtggtgcatggttgtcgtcagctcgtgtcgtgagatgttgggttaagtcccgcaacgagcgcaacccttgatcttagttgccatcattwagttgggcactctaaggtgactgccggtgacaaaccggaggaaggtggggatgacgtcaaatcatcatgccccttatgacctgggctacacacgtgctacaatggacggtacaaagagctgcaagaccgcgaggtggagctaatctcataaaaccgttctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggggtaacctttttggagccagccgcctaaggtgggacagatgattggggtgaagtcgtaacaaggtagccgtatcggaaggtgcggctggatcacctcctttct

SEQ ID NO: 39 - DNA, 1610 bp, Burkholderia pseudomallei
tctagatgcgtgctcgagcggccgcccagtgctgcatggatatctgctgaattcggcttgagcagtttgatcctggctcagattgaacgctggcggcatgccttacacatgcaagtcgaacggcagcacgggcttcggcctggtggcgagtggcgaacgggtgagttatacatcggagcatgtcctgtagtgggggatagcccggcgaaagccgaattaataccgcatacgatctgaggatgaaagcgggggaccttcgggcctcgcgctatagggttggccgatggctgattagctagttggtggggtaaaggcctaccaaggcgacgatcagtagctggtctgagaggacgaccagccacactgggactgagacacggcccagactcctacgggaggcagcagtggggaattttggacaatgggcgcaagcctgatccagcaatgccgcgtgtgtgaagaaggccttcgggttgtaaagcacttttgtccggaaagaaatcattctggctaatacccggagtggatgacggtaccggaagaataagcaccggctaactacgtgccagcagccgcggtaatacgtagggtgcgagcgttaatcgggattactgggcgtaaagcgtgcgcaggcggtttgctaagaccgatgtgaaatccccgggctcaacctgggaactgcattggtgactggcaggctagagtatggcagaggggggtagaattccacgtgtagcagtgaaatgcgtagagatgtggaggaataccgatggcgaaggcagccccctgggccaatactgacgctcatgcacgaaagcgtggggagaaaacaggattagataccctggtagtccacgccctaaacgatgtcaactagttgttggggattcatttccttagtaacgtagctaacgcgcgaagttgaccgcctggggagtacggtcgcaagattaaaactcaaaggaattgacggggacccgcacaagcggtggatgatgtggattaattcgatgcaacgcgaaaaaccttacctacccttgacatggtcggaagcccgatgagagttgggcgtgctcgaaagagaaccggcgcacaggtgctgcatggctgtcgtcagctcgtgtcgtgagatgttgggttaagtcccgcaacgagcgcaacccttgtccttagttgctacgcaagagcactctaaggagactgccggtgacaaaccggaggaaggtggggatgacgtcaagtcctcatggcccttatgggtagggcttcacacgtcatacaatggtcggaacagagggtcgccaacccgcgagggggagccaatcccagaaaaccgatcgtagtccggattgcactctgcaactcgagtgcatgaagctggaatcgctagtaatcgcggatcagcatgccgcggtgaatacgttcccgggtcttgtacacaccgcccgtcacaccatgggagtgggttttaccagaagtggctagtctaaccgcaaggaggacggtcaccacggtaggattcatgactggggtgaagtcgtaacaaggtagccgtagaagccgaattccagcacactggcggccgttactactggatccgagctcgtacc

SEQ ID NO: 40 - DNA, 1511 bp, Clostridium botulinum
  misc_feature (1)..(7): n is a, c, g, or t
nnnnnnngagagtttgatcctggctcaggacgaacgctggcggcgtgcttaacacatgcaagtcgagcgatgaagcttccttcgggaagtggattagcggcggacgggtgagtaacacgtgggtaacctgcctcaaagtgggggatagccttccgaaaggaagattaataccgcataatataagagaatcgcatgattttcttatcaaagatttattgctttgagatggacccgcggcgcattagctagttggtaaggtaacggcttaccaaggcaacgatgcgtagccgacctgagagggtgatcggccacattggaactgagacacggtccagactcctacgggaggcagcagtggggaatattgcgcaatgggggagaccctgacgcagcaacgccgcgtgggtgatgaaggtcttcggattgtaaagccctgttttctaggacgataatgacggtactagaggaggaagccacggctaactacgtgccagcagccgcggtaatacgtaggtggcgagcgttgtccggatttactgggcgtaaagggtgcgtaggcggatgtttaagtgggatgtgaaatccccgggcttaacctgggggctgcattccaaactggatatctagagtgcaggagaggaaagcggaattcctagtgtagcggtgaaatgcgtagagattaggaagaacaccagtggcgaaggcggctttctggactgtaactgacgctgaggcacgaaagcgtgggtagcaaacaggattagataccctggtagtccacgccgtaaacgatggatactaggtgtagggggtatcaactccccctgtgccgcagttaacacaataagtatcccgcctggggagtacggtcgcaagattaaaactcaaaggaattgacgggggcccgcacaagcagcggagcatgtggtttaattcgaagcaacgcgaagaaccttacctggacttgacatcccttgcatagcctagagataggtgaagcccttcggggcaaggagacaggtggtgcatggttgtcgtcagctcgtgtcgtgagatgttaggttaagtcctgcaacgagcgcaacccttgttattagttgctaccattaagttgagcactctaatgagactgcctgggtaaccaggaggaaggtggggatgacgtcaaatcatcatgccccttatgtccagggctacacacgtgctacaatggtaggtacaataagacgcaagaccgtgaggtggagcaaaacttataaaacctatctcagttcggattgtaggctgcaactcgcctacatgaagctggagttgctagtaatcgcgaatcagaatgtcgcggtgaatacgttcccgggccttgtacacaccgccccgtcacaccatgagagctggtaacacccgaagtccgtgaggtaaccgtaaggagccagcggccgaaggtgggattagtgattggggtgaagtcgtaacaaggtagccgtaggagaacctgcggctggatcacctcc

SEQ ID NO: 41 - DNA, 1510 bp, Clostridium botulinum
  misc_feature (1)..(7): n is a, c, g, or t
nnnnnnngagagtttgatcctggctcaggacgaacgctggcggcgtgcttaacacatgcaagtcgagcgatgaagcttccttcgggaagtggattagcggcggacgggtgagtaacacgtgggtaacctgcctcaaagtgggggatagccttccgaaaggaagattaataccgcataacataagagaatcgcatgattttcttatcaaagatttattgctttgagatggacccgcggcgcattagctagttggtaaggtaacggcttaccaaggcaacgatgcgtagccgacctgagagggtgatcggccacattggaactgagacacggtccagactcctacgggaggcaggagtggggaatattgcgcaatgggggaaaccctgacgcagcaacgccgcgtgggtgatgaaggtcttcggattgtaaagccctgttttctaggacgataatgacggtactagaggaggaagccacggctaactacgtgccagcagccgcggtaatacgtaggtggcgagcgttgtccggatttactgggcgtaaagggtgcgtaggcggatgtttaagtgggatgtgaaatccccgggcttaacctgggggctgcattccaaactggatatctagagtgcaggagaggaaagcggaattcctagtgtagcggtgaaatgcgtagagattaggaagaacaccagtggcgaaggcggctttctggactgtaactgacgctgaggcacgaaagcgtgggtagcaaacaggattagataccctggtagtccacgccgtaaacgatggatactaggtgtagggggtatcaactccccctgtgccgcagttaacacaataagtatcccgcctggggagtacggtcgcaagattaaaactcaaaggaattgacgggggcccgcacaagcagcggagcatgtggtttaattcgaagcaacgcgaagaaccttacctggacttgacatcccttgcatagcctagagataggtgaagcccttcggggcaaggagacaggtggtgcatggttgtcgtcagctcgtgtcgtgagatgttaggttaagtcctgcaacgagcgcaacccttgttattagttgctaccattaagttgagcactctaatgagactgcctgggtaaccaggaggaaggtggggatgacgtcaaatcatcatgccccttatgtccagggctacacacgtgctacaatggtaggtacaataagacgcaagaccgtgaggtggagcaaaacttataaaacctatctcagttcggattgtaggctgcaactcgcctacatgaagctggagttgctagtaatcgcgaatcagaatgtcgcggtgaatacgttcccgggccttgtacacaccgcccgtcacaccatgagagctggtaacacccgaagtccgtgaggtaaccgtaaggagccagcggccgaaggtgggattagtgattggggtgaagtcgtaacaaggtagccgtaggagaacctgcggctggatcacctcc

SEQ ID NO: 42 - DNA, 1516 bp, Clostridium botulinum
  misc_feature (1)..(7): n is a, c, g, or t
nnnnnnngagagtttgatcctggctcaggacgaacgtggcggcgtgcctaacacatgcaagtcgagcgatgaagcttccttcggggagtggattagcggcggacgggtgagtaacacgtgggtaacctgcctcaaagagggggatagcctcccgaaagggagattaataccgcataacattattttatggcatcatagaataatcaaaggagcaatccgctttgattatggacccgcgtcgcattagctagttggtgaggtaacggctcaccaaggcaacgatgcgtagccgacctgagagggtgatcggccacattggaactgagacacggtccagactcctacgggaggcagcagtggggaatattgcgcaatgggggaaaccctgacgcagcaacgccgcgtgagtgatgaaggttttcggatcgtaaaactctgtctttagggacgataatgacggtacctaaggaggaagccacggctaactacgtgccagcagccgcggtaatacgtaggtggcaagcgttgtccggatttactgggcgtaaagagtatgtaggtgggtgcttaagtcagatgtgaaattcccgggcttaacctgggcgctgcatttgaaactgggcatctagagtgcaggagaggaaagtggaattcctagtgtagcggtgaaatgcgtagagattaggaagaacaccagtggcgaaggcgactttctggactgtaactgacactgagatacgaaagcgtgggtagcaaacaggattagatccccctggtagtccacgccgtaaacgatgaatactaggtgtcggggggtaccaccctcggtgccgcagcaaacgcattaagtattccgcctggggagtacggtcgcaagattaaaactcaaaggaattgacggggacccgcacaagcagcggagcatgtggtttaattcgaagcaacgcgaagaaccttacctagacttgacatctcctgaattactcttaatcgaggaagtcccttcgggggacaggaagacaggtggtgcatggttgtcgtcagctcgtgtcgtgagatgttgggttaagtcccgcaacgagcgcaacccttattgttagttgctactattaagttaagcactctaacgagactgccgcggttaacgtagaggaaggtggggatgacgtcaaatcatcatgccccttatgtctagggctacacacgtgctacaatggctggtacaacgagcagcaaacccgcgagggggagcaaaacttgaaagccagtcccagttcggattgtaggctgaaactcgcctacatgaagttggagttgctagtaatcgcggaatcagcatgtcgcggtgaatacgtccccgggtcttgtacacaccgcccgtcacaccatgagagccggtaacacccgaagcccgtgaggtaaccgtaaggagccagcggtcgaaggtgggattggtgattggggtgaagtcgtaacaaggtagccgtaggagaacctgcggttggatcacctcctt

SEQ ID NO: 43 - DNA, 1516 bp, Clostridium botulinum
  misc_feature (1)..(7): n is a, c, g, or t
  misc_feature (373)..(373): n is a, c, g, or t
  misc_feature (789)..(790): n is a, c, g, or t
nnnnnnngagagtttgatcctggctcaggacgaacgctggcggcgtgcctaacacatgcaagtcgagcgatgaagcttccttcgggaagtggattagcggcggacgggtgagtaacacgtgggtaacctgcctcaaagagtgggatagcctcccgaaagggagattaataccgcataacattattttatggcatcatacataaaataatcaaaggagcaatccgctttgagatggacccgcggcgcattagctagttggtgaggtaacggctcaccaaggcaacgatgcgtagccgacctgagagggtgatcggccacattggaactgagacacggtccagactcctacgggaggcagcagtggggaatattncgcaatgggggaaaccctgacgcagcaacgccgcgtgagtgatgaaggttttcggatcgtaaaactctgtctttagggacgataatgacggtacctaaggaggaagccacggctaactacgtgccagcagccgcggtaatacgtaggtggcaagcgttgtccggatttactgggcgtaaagagtatgtaggtgggtgcttaagtcagatgtgaaattcccgggctcaacctgggagctgcatttgaaactgggcatctagagtgcaggagaggaaagtggaattcctagtgtagcggtgaaatgcgtagagattaggaagaacaccagtggcgaaggcgactctctggactgtaactgacactgagatacgaaagcgtgggtagcaaacaggattagataccctggtagtccacgnngtaaacgatgaatactaggtgtcggggggtaccaccctcggtgccgcagcaaacgcattaagtattccgcctgggaagtacggtcgcaagattaaaactcaaaggaattgacggggcccgcacaagcagcggagcatgtggtttaattcgaagcaacgcgaagaaccttacctagacttgacatctcctgaattactcttaatcgaggaagtcccttcggggacaggaagacaggtggtgcatggttgtcgtcagctcgtgtcgtgagatgttgggttaagtcccgcaacgagcgcaacccttattgttagttgctactattaagttaagcactctaacgagactgccgcggttaacgtggaggaaggtggggatgacgtcaaatcatcatgccccttatgtctagggctacacacgtgctacaatggctggtacaacgagcagcaaacccgcgagggggagcaaaacttgaaagccagtcccagttcggattgtaggctgaaactcgcctacatgaagttggagttgctagtaatcgcgaatcagcatgtcgcggtgaatacgttcccgggtcttgtacacaccgcccgtcacaccatgagagccggtaacacccgaagcccgtgaggtaaccgtaaggagccagcggtcgaaggtgggattggtgattggggtaagtcgtaacaaggtagccgtaggagaacctgcggctggatcacctccttt

SEQ ID NO: 44 - DNA, 1513 bp, Clostridium botulinum
  misc_feature (1)..(7): n is a, c, g, or t
nnnnnnnagagtttgatcctggctcaggacgaacgctggcggcgtgcctaacacatgcaatcgagcgatgaagcttccttcgggaagtggattagcggcggacgggtgagtaacacgtgggtaacctgcctcatagaggggaatagcctcccgaaagggagattaataccgcataaagtatgaaggtcgcatgacttcattataccaaaggagtaatccgctatgagatggacccgcggcgcattagctagttggtgaggtaagggctcaccaaggcaacgatgcgtagccgacctgagagggtgatcggccacattggaactgagacacggtccagactcctacgggaggcagcagtggggaatattgcgcaatgggggaaaccctgacgcagcaacgccgcgtgaatgaagaaggccttagggttgtaaagttctgtcatatgggaagataatgacggtaccatatgaggaagccacggctaactacgtgccagcagccgcggtaatacgtaggtggcaagcgttgtccggatttactgggcgtaaaggatgcgtaggcggacatttaagtcagatgtgaaatacccgggctcaacttgggtgctgcatttgaaactgggtgtctagagtgcaggagaggaaagcggaattcctagtgtagcggtgaaatgcgtagagattaggaagaacaccagtggcgaaggcggctttctggactgtaactgacgctgaggcatgaaagcgtggggagcaaacaggattagataccctggtagtccacgccgtaaacgatgaatactaggtgtaggaggtatcgaccccttctgtgccgcagttaacacaataagtattccgcctggggagtacgatcgcaagattaaaactcaaaggaattgacgggggcccgcacaagcagcggagcatgtggtttaattcgaagcaacgcgaagaaccttacctagacttgacatcccctgaattacctgtaatgagggaagcccttcggggcagggagacaggtggtgcatggttgtcgtcagctcgtgtcgtgagatgttgggttaagtcccgcaacgagcgcaacccttatcattagttgctaccattaagttgagcactctagtgagactgcccgggttaaccgggaggaaggtggggatgacgtcaaatcatcatgccccttatgtctagggctacacacgtgctacaatggttggtacaacaagatgcaagaccgcgaggtggagctaaacttaaaaaaccaacccagttcggattgtaggctgaaactcgcctacatgaagccggagttgctagtaatcgcgaatcagcatgtcgcggtgaatacgttcccgggccttgtacacaccgcccgtcacaccatgagagctggtaacacccgaagtccgtgaggtaaccgtaaggagccagcggccgaaggtgggattagtgattggggtgaagtcgtaacaaggtagccgtaggagaacctgcggttggatcacctcctt

SEQ ID NO: 45 - DNA, 1521 bp, Francisella tularensis
ttgaagagtttgatcatggctcagattgaacgctggtggcatgcttaacacatgcaagtcgaacggtaacaggtcttaggatgctgacgagtggcggacgggtgagtaacgcgtaggaatctgcccatttgagggggataccagttggaaacgactgttaataccgcataatatctgtggattaaaggtggctttcggcgtgtcgcagatggatgagcctgcgttggattagctagttggtggggtaagggcccaccaaggctacgatccatagctgatttgagaggatgatcagccacattgggactgagacacggcccaaactcctacgggaggcagcagtggggaatattggacaatgggggcaaccctgatccagcaatgccatgtgtgtgaagaaggccctagggttgtaaagcactttagttggggaggaaagcctcaaggttaatagccttgggggaggacgttacccaaagaataagcaccggctaactccgtgccagcagccgcggtaatacggggggtgcaagcgttaatcggaattactgggcgtaaagggtctgtaggtggtttgttaagtcagatgtgaaagcccagggctcaaccttggaactgcatttgatactggcaaactagagtacggtagaggaatggggaatttctggtgtagcggtgaaatgcgtagagatcagaaggaacaccaatggcgaaggcaacattctggaccgatactgacactgagggacgaaagcgtggggatcaaacaggattagataccctggtagtccacgctgtaaacgatgagtactagctgttggagtcggtgtaaaggctctagtggcgcacgtaacgcgataagtactccgcctggggactacggccgcaaggctaaaactcaaaggaattgacggggacccgcacaagcggtggagcatgtggtttaattcgatgcaacgcgaagaaccttacctggtcttgacatcctgcgaactttctagagatagattggtgcttcggaacgcagtgacagtgctgcacggctgtcgtcagctcgtgttgtgaaatgttgggttaagtcccgcaacgagcgcaacccctattgatagttaccatcattaagttgggtactctattaagactgccgctgacaaggcggaggaaggtggggacgacgtcaagtcatcatggcccttacgaccagggctacacacgtgctacaatgggtattacagagggctgcgaaggtgcgagctggagcgaaactcaaaaaggtactcttagtccggattgcagtctgcaactcgactgcatgaagtcggaatcgctagtaatcgcaggtcagaatactgcggtgaatacgttcccgggtcttgtacacaccgcccgtcacaccatgggagtgggttgctccagaagtagatagcttaacgaatgggcgtttaccacggagtgattcatgactggggtgaagtcgtaacaatggtagccgtagggaacctgcggctggatcacctcctt

SEQ ID NO: 46 - DNA, 1464 bp, Vibrio cholerae
tggctcagattgaacgctggcggcaggcctaacacatgcaagtcgagcggcagcacagaggaacttgttccttgggtggcgagcggcggacgggtgagtaatgcctgggaaattgcccggtagagggggataaccattggaaacgatggctaataccgcataacctcgtaagagcaaagcaggggaccttcgggccttgcgctaccggatatgcccaggtgggattagctagttggtgaggtaagggctcaccaaggcgacgatccctagctggtctgagaggatgatcagccacactggaactgagacacggtccagactcctacgggaggcagcagtggggaatattgcacaatgggcgcaagcctgatgcagccatgccgcgtgtatgaagaaggccttcgggttgtaaagtactttcagtagggaggaaggtggttaagctaataccttaatcatttgacgttacctacagaagaagcaccggctaactccgtgccagcagccgcggtaatacggagggtgcaagcgttaatcggaattactgggcgtaaagcgcatgcaggtggtttgttaagtcagatgtgaaagccctgggctcaacctaggaatcgcatttgaaactgacaagctagagtactgtagaggggggtagaatttcaggtgtagcggtgaaatgcgtagagatctgaaggaataccggtggcgaaggcggccccctggacagatactgacactcagatgcgaaagcgtggggagcaaacaggattagataccctggtagtccacgccgtaaacgatgtctacttggaggttgtgacctagagtcgtggctttcggagctaacgcgttaagtagaccgcctggggagtacggtcgcaagattaaaactcaaatgaattgacgggggcccgcacaagcggtggagcatgtggtttaattcgatgcaacgcgaagaaccttacctactcttgacatcctcagaagagactggagacagtcttgtgccttcgggaactgagagacaggtgctgcatggctgtcgtcagctcgtgttgtgaaatgttgggttaagtcccgcaacgagcgcaacccttatccttgtttgccagcacgtaatggtgggaactccagggagactgccggtgataaaccggaggaaggtggggacgacgtcaagtcatcatggcccttacgagtagggctacacacgtgctacaatggcgtatacagagggcagcgataccgcgaggtggagcgaatctcacaaagtacgtcgtagtccggattggagtctgcaactcgactccatgaagtcggaatcgctagtaatcgcaaatcagaatgttgcggtgaatacgttcccgggccttgtacacaccgcccgtcacaccatgggagtgggctgcaaaagaagcaggtagtttaaccttcgggaggacgcttgccactttgggtacttgg

SEQ ID NO: 47 - DNA, 1469 bp, Yersinia pestis
ctggcggcaggcctaacacatgcaagtcgagcggcaccgggaagtagtttactactttgccggcgagcggcggacgggtgagtaatgtctggggatctgcctgatggagggggataactactggaaacggtagctaataccgcatgacctcgcaagagcaaagtgggggaccttagggcctcacgccatcggatgaacccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcgaggaggaaggggttgagtttaatacgctcaatcattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgcgcttaacgtgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccacagaatttggcagagatgctaaagtgccttcgggaactgtgagacaggtgctgcatggctgtcgtcagctcgtgttgtgaaatgttgggttaagtcccgcaacgagcgcaacccttatcctttgttgccagcacgtaatggtgggaactcaagggagactgccggtgacaaaccggaggaaggtggggatgacgtcaagtcatcatggcccttacgagtagggctacacacgtgctacaatggcagatacaaagtgaagcgaactcgcgagagccagcggaccacataaagtctgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaa

SEQ ID NO: 48 - DNA, 23 bp, Artificial Sequence
  Forward primer
tcctacgggaggcagcagtaggg

SEQ ID NO: 49 - DNA, 21 bp, Artificial Sequence
  Reverse primer
ccgctacacatggaattccac

SEQ ID NO: 50 - DNA, 25 bp, Artificial Sequence
  Forward primer
gactcctacgggaggcagcagtggg

SEQ ID NO: 51 - DNA, 26 bp, Artificial Sequence
  Reverse primer
ggtattaacttactgcccttcctccc